(19) United States
(12) Patent Application Publication
(10) Pub. No.: US 2026/0274843 A1
(43) Pub. Date: Sep. 17, 2026

(54) SALT AND SOLID FORMS OF A CDK INHIBITOR

(71) Applicant: GENENTECH, INC., South San Francisco, CA (US)

(72) Inventors: Xiaotian ZHU, Newton, MA (US); Wenge ZHONG, Thousand Oaks, CA (US); Hu HE, Shanghai (CN); Zhilong HU, Shanghai (CN); Xiaomei DONG, Beijing (CN); Meiting SHI, Beijing (CN); Ruiping WANG, Beijing (CN)

(73) Assignee: GENENTECH, INC., South San Francisco, CA (US)

(21) Appl. No.: 19/665,654

(22) Filed: May 1, 2026

Related U.S. Application Data
(63) Continuation of application No. PCT/CN2024/132256, filed on Nov. 15, 2024.

(30) Foreign Application Priority Data
Nov. 17, 2023 (WO) PCT/CN2023/132310

(51) Int. Cl. C07D 471/04; A61K 31/506; A61K 45/06; C07C 57/145; C07C 309/30

(52) U.S. Cl. CPC C07D 471/04; A61K 31/506; A61K 45/06; C07C 57/145; C07C 309/30; C07B 2200/13

(57) ABSTRACT

Various salt forms and free base of Compound (I) represented by the following formula are disclosed.

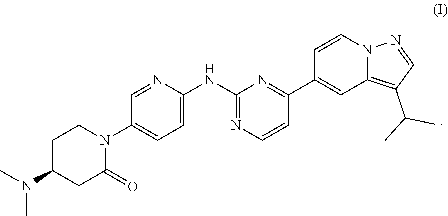 [SMILES: CC(C)c1cnn2ccc(-c3ccnc(Nc4ccc(N5CC[C@H](N(C)C)CC5=O)cn4)n3)cc12]

### RELATED APPLICATIONS

This application claims priority to International Application No. PCT/CN2023/132310, filed on Nov. 17, 2023. The entire contents of the foregoing application are expressly incorporated herein by reference.

### BACKGROUND

Cyclin-Dependent Kinases (CDKs) are a family of protein kinases first discovered for their roles in regulating cell cycle. They have since been identified to play roles in regulating a number of other biological functions such as transcription, mRNA processing, and the differentiation of nerve cells.

CDKs are relatively small proteins with molecular weights between about 34-40 kDa. They contain little more than the kinase domain, and are essentially inactive when not in complex with a class of regulatory proteins called cyclins. CDK levels remain relatively constant throughout the cell cycle, and most regulation is post-translational, most prominently by binding to cyclins.

Like all kinases, the active site, or the ATP-binding site, of CDKs is a cleft between a small amino-terminal lobe and a larger carboxy-terminal lobe. The structure of human CDK2 revealed that CDKs have a modified ATP-binding site that can be regulated by cyclin binding. Phosphorylation by CDK-activating kinase (CAK) at Thr 161 on the T-loop increases the complex activity. Without cyclin, a flexible loop called the activation loop or T-loop blocks the cleft, and the position of several key amino acid residues is not optimal for ATP-binding. With cyclin, two alpha helices change position to permit ATP binding. One of them, the L12 helix that comes just before the T-loop in the primary sequence, becomes a beta strand and helps rearrange the T-loop, so it no longer blocks the active site. The other alpha helix called the PSTAIRE helix rearranges and helps change the position of the key amino acid residues in the active site.

Thus only the cyclin-CDK complex has active kinase activity, and most of known cyclin-CDK complexes regulate the progression through the cell cycle. The CDKs are ubiquitous in all known eukaryotes, and their regulatory function in the cell cycle has been evolutionarily conserved. For example, yeast cells can proliferate normally when their CDK gene has been replaced with the homologous human gene. CDKs exert their regulatory function by phosphorylating their substrates on certain specific Serine and Threonine residues, and the consensus sequence of [S/T]PX[K/R], where S/T is the target Ser or Thr for phosphorylation, P is proline, X is any amino acid, K is lysine, and R is arginine.

In animal cells, there are at least nine different CDKs, four of which (CDK1, 2, 3, and 4) are directly involved in cell cycle regulation. In mammalian cells, CDK1, with its binding partners cyclin A2 and B1, alone can drive the cell cycle. Cyclin-CDK complexes of earlier cell-cycle phase can help to activate cyclin-CDK complexes in later phase.

The same CDK may form complexes with different cyclins to regulate different phases of the cell cycle. For example, CDK2 may form a complex with cyclin D or E to regulate G1 phase; form a complex with cyclin A or E to regulate S phase; and form a complex with cyclin A to regulate G2 phase. Meanwhile, CDK4 and CDK6 can form complexes with cyclins D1, D2, and D3.

The highly homologous Cyclin-dependent kinases (CDKs) CDK4 and CDK6 in combination with Cyclin D are key regulators of the transition through the restriction point R between the G1 (growth) and S (DNA replication) phases of the cell cycle. CDK4/6 exert their effects via phosphorylation of the retinoblastoma protein (pRb). Once phosphorylated, pRb loses its inhibitory effect on the transcription of genes promoting entry into S phase.

By contrast, specific inhibition of CDK4/6 kinase activity by the endogenous protein modulator p16INK4 or by small molecule inhibitors results in hypophosphorylated pRb and arrest of the cells at the G1 restriction point. As the primary mechanism of regulating the G1 restriction point, the pathway regulated by these kinases is altered in a broad spectrum of human tumors, and thus inhibition of CDK4/CDK6 in these tumors has therapeutic benefit by preventing cell division.

International Publication No. WO 2020/224568, the entire teachings of which are incorporated herein by references discloses highly potent, high selective inhibitors of CDKs, including CDK2, CDK4, and CDK6. The structure of one of the inhibitors disclosed in International Publication No. WO 2020/224568, referred to herein as “Compound (I)” is shown below:

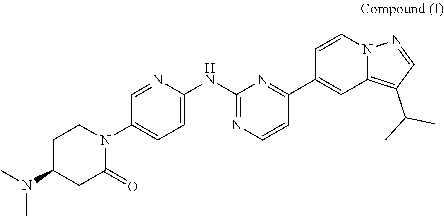 [SMILES: CC(C)c1cnn2ccc(-c3ccnc(Nc4ccc(N5CC[C@H](N(C)C)CC5=O)cn4)n3)cc12]

Compound (I) is a potent, selective, small molecule inhibitor of CDK2, CDK4, and CDK6.

The successful development of pharmaceutically active agents, such as Compound (I), typically requires the identification of a solid form with properties that enable ready isolation and purification following synthesis, that are amendable to large scale manufacture, that can be stored for extended periods of time with minimal absorption of water, decomposition or transformation into other solid forms, that are suitable for formulation and that can be readily absorbed following administration to the subject (e.g., are soluble in water and in gastric fluids).

### SUMMARY

Described herein are polymorph forms of the free base of Compound (I) and salt forms of Compound (I) that inhibit the activity of a cyclin-dependent kinase (CDK), e.g., CDK2, CDK4, and/or CDK6, and pharmaceutically acceptable salts, or stereoisomers thereof.

It has now been found that the free base of Compound (I) can be crystallized under well-defined conditions to afford seven different crystalline forms, including five anhydrates (Form I, II, III, IV and VII), a hemi-hydrate (Form VI), a solvate of toluene with 1 mol (Form V). Among all forms, Form II has high crystallinity, high melting point of 191° C. and slight hygroscopicity. The inter-conversion study (Example 2) showed that all mixtures transformed to Form II at RT and 50° C. or 60° C., which suggested Form II has very good physical stability. The accelerated and long term stability study (Example 3) indicated Form II was physically stable at room temperature (RT)/92.5% RH, and physically and chemically stable at 40° C./75% RH and 60° C. for 10 days. Form II was the most stable anhydrous form with favorable solid state properties. Form II proved stable at the temperatures below 60° C. in non-aqueous solvent, and physically stable in aqueous solvent with less than 5% of water. In short, Form II possesses a favorable overall balance of properties which makes it especially suitable for pharmaceutical formulation and use.

Additionally, Compound (I) can form a salt upon the addition of a variety of acids. In salt screening, total 12 salts forms were identified. Therein, tosylate Form I and maleate Form I with better solid state properties were selected as lead salts for solubility and stability studies. The solubility of these samples showed obvious pH dependency. Tosylate Form I and maleate Form I have high solubility in water. They showed similar solubility in SGF (>5 mg/mL) and in FeSSIF (2-3 mg/mL). In FaSSIF, the solubility of maleate Form I was ~30 folds of that of tosylate Form I. Maleate Form I had relatively higher solubility than others. Tosylate Form I and maleate Form I was physically stable under two conditions. However, the purities both decreased by 0.1% at 40° C./75% RH and 0.5% at 60° C. for 24 days.

The present disclosure provides a method of treating a disease or condition in a patient in need thereof, comprising administering to a patient in need thereof the salt or the free base (including both amorphous form and crystalline form) of Compound (I) disclosed herein, or the corresponding pharmaceutical composition thereof, wherein the disease or condition is chosen from cancer In certain embodiments, the cancer is selected from the group consisting of colorectal cancer, breast cancer (such as hormone receptor positive, HER2/neu negative advanced or metastatic breast cancer in postmenopausal women), lung cancer, prostate cancer, glioblastoma, mantel cell lymphoma, chronic myeloid leukemia and acute myeloid leukemia.

In certain embodiments of the methods of the invention, the cancer can be treated by inhibiting the activity of a cyclin-dependent kinase (CDK), e.g., CDK2, CDK4, and/or CDK6.

In certain embodiments of the methods of the invention, the cancer is carcinoma of the bladder, breast, colon, kidney, epidermis, liver, lung, oesophagus, gall bladder, ovary, pancreas, stomach, cervix, thyroid, nose, head and neck, prostate, or skin; a hematopoietic tumor of lymphoid lineage; a hematopoietic tumor of myeloid lineage; thyroid follicular cancer; a tumor of mesenchymal origin; a tumor of the central or peripheral nervous system; melanoma; seminoma; teratocarcinoma; osteosarcoma; xeroderma pigmentosum; keratoctanthoma; thyroid follicular cancer; or Kaposi's sarcoma.

In certain embodiments of the methods of the invention, the compounds of the invention are administered with any one of a second therapeutic agent as described herein that also treats the same cancer.

The present disclosure also provides a use of the salt or freebase of Compound (I) of the disclosure or a pharmaceutical composition thereof comprising the same for the treatment of any of the disease recited in the previous paragraph. In one embodiment, provided is the salt or freebase of the disclosure or a pharmaceutical composition thereof comprising the same for use in any of the method of the disclosure described herein. In another embodiment, provided is use of the salt of the disclosure or freebase or a pharmaceutical composition thereof comprising the same for the manufacture of a medicament for any of the method of the disclosure described.

### BRIEF DESCRIPTION OF THE DRAWINGS

FIG. 1A shows the X-ray Powder Diffraction (XRPD) pattern of crystalline Form I of Compound (I).

FIG. 1B shows the Thermogravimetric Analysis (TGA) and Differential Scanning Calorimetry Analysis (DSC) thermograms of crystalline Form I of Compound (I).

FIG. 2A shows the X-ray Powder Diffraction (XRPD) pattern of crystalline Form II of Compound (I).

FIG. 2B shows the Thermogravimetric Analysis (TGA) and Differential Scanning Calorimetry Analysis (DSC) thermograms of crystalline Form II of Compound (I).

FIG. 3A shows the X-ray Powder Diffraction (XRPD) pattern of crystalline Form III of Compound (I).

FIG. 3B shows the Thermogravimetric Analysis (TGA) and Differential Scanning Calorimetry Analysis (DSC) thermograms of crystalline Form III of Compound (I).

FIG. 4A shows the X-ray Powder Diffraction (XRPD) pattern of crystalline Form IV of Compound (I).

FIG. 4B shows the Thermogravimetric Analysis (TGA) and Differential Scanning Calorimetry Analysis (DSC) thermograms of crystalline Form IV of Compound (I).

FIG. 5A shows the X-ray Powder Diffraction (XRPD) pattern of crystalline Form V of Compound (I).

FIG. 5B shows the Thermogravimetric Analysis (TGA) and Differential Scanning Calorimetry Analysis (DSC) thermograms of crystalline Form V of Compound (I).

FIG. 6A shows the X-ray Powder Diffraction (XRPD) pattern of crystalline Form VI of Compound (I).

FIG. 6B shows the Thermogravimetric Analysis (TGA) and Differential Scanning Calorimetry Analysis (DSC) thermograms of crystalline Form VI of Compound (I).

FIG. 7A shows the X-ray Powder Diffraction (XRPD) pattern of crystalline Form VII of Compound (I).

FIG. 7B shows the Thermogravimetric Analysis (TGA) and Differential Scanning Calorimetry Analysis (DSC) thermograms of crystalline Form VII of Compound (I).

FIG. 8A shows the X-ray Powder Diffraction (XRPD) pattern of crystalline Form I of maleate salt of Compound (I).

FIG. 8B shows the Thermogravimetric Analysis (TGA) and Differential Scanning Calorimetry Analysis (DSC) thermograms of crystalline Form I of maleate salt of Compound (I).

FIG. 9A shows the X-ray Powder Diffraction (XRPD) pattern of crystalline Form I of tosylate salt of Compound (I).

FIG. 9B shows the Thermogravimetric Analysis (TGA) and Differential Scanning Calorimetry Analysis (DSC) thermograms of crystalline Form I of tosylate salt of Compound (I).

FIG. 10A shows the X-ray Powder Diffraction (XRPD) pattern of crystalline Form I of HCl salt of Compound (I).

FIG. 10B shows the Thermogravimetric Analysis (TGA) and Differential Scanning Calorimetry Analysis (DSC) thermograms of crystalline Form I of HCl salt of Compound (I).

FIG. 11A shows the X-ray Powder Diffraction (XRPD) pattern of crystalline Form II of HCl salt of Compound (I).

FIG. 11B shows the Thermogravimetric Analysis (TGA) and Differential Scanning Calorimetry Analysis (DSC) thermograms of crystalline Form II of HCl salt of Compound (I).

FIG. 12A shows the X-ray Powder Diffraction (XRPD) pattern of crystalline Form III of HCl salt of Compound (I).

FIG. 12B shows the Thermogravimetric Analysis (TGA) and Differential Scanning Calorimetry Analysis (DSC) thermograms of crystalline Form III of HCl salt of Compound (I).

FIG. 13A shows the X-ray Powder Diffraction (XRPD) pattern of crystalline Form IV of HCl salt of Compound (I).

FIG. 13B shows the Thermogravimetric Analysis (TGA) and Differential Scanning Calorimetry Analysis (DSC) thermograms of crystalline Form IV of HCl salt of Compound (I).

FIG. 14A shows the X-ray Powder Diffraction (XRPD) pattern of crystalline Form I of HBr salt of Compound (I).

FIG. 14B shows the Thermogravimetric Analysis (TGA) and Differential Scanning Calorimetry Analysis (DSC) thermograms of crystalline Form I of HBr salt of Compound (I).

FIG. 15A shows the X-ray Powder Diffraction (XRPD) pattern of crystalline Form I of sulfate salt of Compound (I).

FIG. 15B shows the Thermogravimetric Analysis (TGA) and Differential Scanning Calorimetry Analysis (DSC) thermograms of crystalline Form I of sulfate salt of Compound (I).

FIG. 16A shows the X-ray Powder Diffraction (XRPD) pattern of crystalline Form I of phosphate salt of Compound (I).

FIG. 16B shows the Thermogravimetric Analysis (TGA) and Differential Scanning Calorimetry Analysis (DSC) thermograms of crystalline Form I of phosphate salt of Compound (I).

FIG. 17A shows the X-ray Powder Diffraction (XRPD) pattern of crystalline Form I of tartrate salt of Compound (I).

FIG. 17B shows the Thermogravimetric Analysis (TGA) and Differential Scanning Calorimetry Analysis (DSC) thermograms of crystalline Form I of tartrate salt of Compound (I).

FIG. 18A shows the X-ray Powder Diffraction (XRPD) pattern of crystalline Form I of fumarate salt of Compound (I).

FIG. 18B shows the Thermogravimetric Analysis (TGA) and Differential Scanning Calorimetry Analysis (DSC) thermograms of crystalline Form I of fumarate salt of Compound (I).

FIG. 19A shows the X-ray Powder Diffraction (XRPD) pattern of crystalline Form II of fumarate salt of Compound (I).

FIG. 19B shows the Thermogravimetric Analysis (TGA) and Differential Scanning Calorimetry Analysis (DSC) thermograms of crystalline Form II of fumarate salt of Compound (I).

FIG. 20A shows the X-ray Powder Diffraction (XRPD) pattern of crystalline Form I of malate salt of Compound (I).

FIG. 20B shows the Thermogravimetric Analysis (TGA) and Differential Scanning Calorimetry Analysis (DSC) thermograms of crystalline Form I of malate salt of Compound (I).

FIG. 21A shows the X-ray Powder Diffraction (XRPD) pattern of crystalline Form I of succinate salt of Compound (I).

FIG. 21B shows the Thermogravimetric Analysis (TGA) and Differential Scanning Calorimetry Analysis (DSC) thermograms of crystalline Form I of succinate salt of Compound (I).

FIG. 22 shows a schematic depiction of the relationships between different forms related the interconversion studies.

FIG. 23 shows a crystal structure of Compound (I) indicating S-configuration.

### DETAILED DESCRIPTION

The present disclosure is directed to: i) novel crystalline forms of Compound (I), including unsolvated forms, solvated forms, and crystalline forms; ii) novel solid forms of pharmaceutically acceptable salts of Compound (I), including unsolvated forms, solvated forms, amorphous forms, and crystalline forms (hereinafter collectively referred to as “salt forms”); and iii) methods of use and preparation of the crystalline forms and salt forms of Compound (I).

As used herein, “crystalline” refers to a solid having a crystal structure wherein the individual molecules have a highly homogeneous regular three dimensional configuration.

In one aspect, the present disclosure provides crystalline forms of Compound (I). The form of Compound (I) can be crystalline and can exist as one or more polymorph forms. These polymorphic forms can be solvated or unsolvated forms. These polymorphic or crystalline forms differ with respect to their X-ray powder diffractions (XRPD) patterns, spectroscopic, physicochemical, and pharmacokinetic properties, as well as their thermodynamic stability.

In another aspect, the present disclosure provides crystalline forms of Compound (I) as salt forms. Many different salt forms can exist depending on the salt used. Each salt forms can exists as one or more polymorph salt forms. The polymorph salt form can be solvated or unsolvated. These polymorphic or crystalline salt forms differ with respect to their X-ray powder diffractions (XRPD) patterns, spectroscopic, physicochemical, and pharmacokinetic properties, as well as their thermodynamic stability.

It is desirable to have access to different polymorphic forms of Compound (I) for several reasons. Distinct polymorph forms may exhibit different physical properties such as melting point, hygroscopicity, solubility, flow properties or thermodynamic stability, and therefore, distinct polymorph forms allow the choice of the most suitable form for a given use or aspect, for example, in distinct administration forms such as capsules, or in the manufacture of a drug form having optimum pharmacokinetic properties.

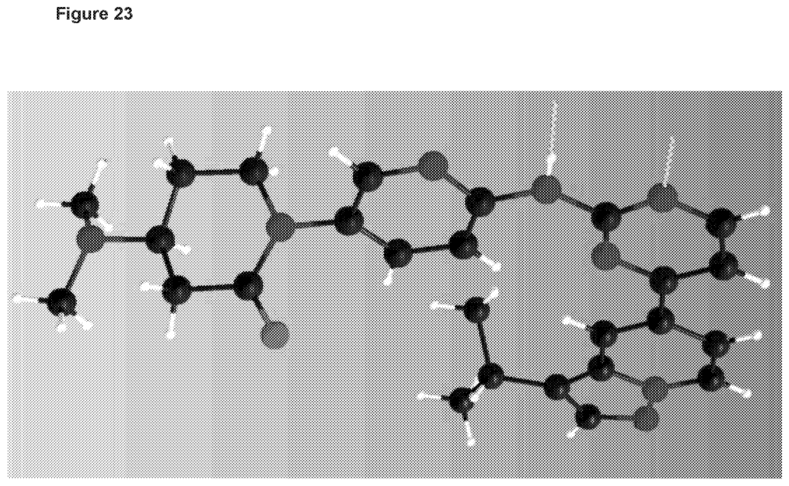

The present invention provides a polymorphic forms of (S)-4-(dimethylamino)-1-(6-((4-(3-isopropylpyrazolo[1,5-a]pyridin-5-yl)pyrimidin-2-yl)amino)pyridin-3-yl)piperidin-2-one, also referred to as “Compound (I)”, originally described in International Publication No. WO 2020/224568, Synthetic Example 161. The contents of WO 2020/224568 is incorporated herewith by reference, in particular the disclosure related to the synthesis, Synthetic Example 161. The S-configuration is confirmed by the crystal structure of Compound (I). See FIG. 23.

It has now been surprisingly found that under certain conditions new solid forms of Compound (I), can be provided which are described hereinafter as Form I, Form II, Form III, Form IV, Form V, Form VI, Form VII, HCl salt Form I, HCl salt Form II, HCl salt Form III, HCl salt Form IV, Sulfate salt Form I, Phosphate salt Form I, Tosylate salt Form I, Maleate salt Form I, Tartrate salt Form I, Fumarate salt Form I, Fumarate salt Form II, and Succinate salt Form I, which have advantageous utilities and properties. In particular, Form II of the Compound (I) shows excellent stability properties when subject to stress conditions. A particular polymorph form of Compound 1, namely Form II, demonstrated high crystallinity and high physical and chemical stability. This high degree of stability of Form II provides advantageous properties and benefits in terms of its suitability for use in a pharmaceutical composition, for example, in terms of its shelf-life and ease of manufacture.

### Crystalline Forms of Compound (I)

### Form I

In one aspect, the present disclosure provides a solid form of Compound (I),

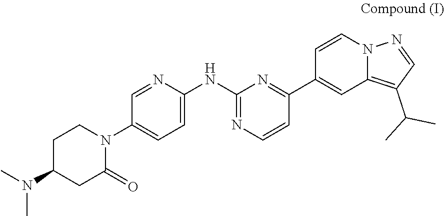 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form I.

In one embodiment the present disclosure provides crystalline Form I of Compound (I), which is characterized by a XRPD diffraction pattern comprising a XRPD peak at an angle of diffraction at about 6.8° (±0.2), 18.4° (±0.2), 21.4° (±0.2), or 24.1° (±0.2) in 2θ.

In another embodiment, the crystalline Form I of Compound (I) is characterized by a XRPD diffraction pattern comprising at least two XRPD peaks selected from XRPD peaks at an angle of diffraction at about 6.8° (±0.2), 18.4° (±0.2), 21.4° (±0.2), and 24.1° (±0.2) in 2θ. In another embodiment, the crystalline Form I of Compound (I) is characterized by a XRPD diffraction pattern comprising XRPD peaks at an angle of diffraction at about 6.8° (±0.2) and 18.4° (±0.2) in 2θ. In another embodiment, the crystalline Form I of Compound (I) is characterized by a XRPD diffraction pattern comprising XRPD peaks at an angle of diffraction at about 6.8° (±0.2) and 21.4° (±0.2) in 2θ. In another embodiment, the crystalline Form I of Compound (I) is characterized by a XRPD diffraction pattern comprising XRPD peaks at an angle of diffraction at about 6.8° (±0.2) and 24.1° (±0.2) in 2θ. In yet another embodiment, the crystalline Form I of Compound (I) is characterized by a XRPD diffraction pattern comprising XRPD peaks at an angle of diffraction at about 18.4° (±0.2) and 21.4° (±0.2) in 2θ. In yet another embodiment, the crystalline Form I of Compound (I) is characterized by a XRPD diffraction pattern comprising XRPD peaks at an angle of diffraction at about 18.4° (±0.2) and 24.1° (±0.2) in 2θ. In yet another embodiment, the crystalline Form I of Compound (I) is characterized by a XRPD diffraction pattern comprising XRPD peaks at an angle of diffraction at about 21.4° (±0.2) and 24.1° (±0.2) in 2θ.

In yet another embodiment, the crystalline Form I of Compound (I) is characterized by a XRPD diffraction pattern comprising at least three XRPD peaks selected from XRPD peaks at an angle of diffraction at about 6.8° (±0.2), 18.4° (±0.2), 21.4° (±0.2), and 24.1° (±0.2) in 2θ. In yet another embodiment, the crystalline Form I of Compound (I) is characterized by a XRPD diffraction pattern comprising XRPD peaks at an angle of diffraction at about 6.8° (±0.2), 18.4° (±0.2), and 21.4° (±0.2) in 2θ. In yet another embodiment, the crystalline Form I of Compound (I) is characterized by a XRPD diffraction pattern comprising XRPD peaks at an angle of diffraction at about 6.8° (±0.2), 18.4° (±0.2), and 24.1° (±0.2) in 2θ. In yet another embodiment, the crystalline Form I of Compound (I) is characterized by a XRPD diffraction pattern comprising XRPD peaks at an angle of diffraction at about 6.8° (±0.2), 21.4° (±0.2), and 24.1° (±0.2) in 2θ. In yet another embodiment, the crystalline Form I of Compound (I) is characterized by a XRPD diffraction pattern comprising XRPD peaks at an angle of diffraction at about 18.4° (±0.2), 21.4° (±0.2), and 24.1° (±0.2) in 2θ.

In yet another embodiment, the crystalline Form I of Compound (I) is characterized by a XRPD diffraction pattern comprising four XRPD peaks at an angle of diffraction at about 6.8° (±0.2), 18.4° (±0.2), 21.4° (±0.2), and 24.1° (±0.2) in 2θ.

In one embodiment, the present disclosure provides crystalline Form I of Compound (I), which is characterized by XRPD peaks at 6.8°, 18.4°, 21.4°, and 24.1°±0.2 in 2θ.

In another embodiment, the present disclosure provides a crystalline Form I of Compound (I), which is characterized by differential scanning calorimeter (DSC) peak phase transition temperature of 211.6±2° C.

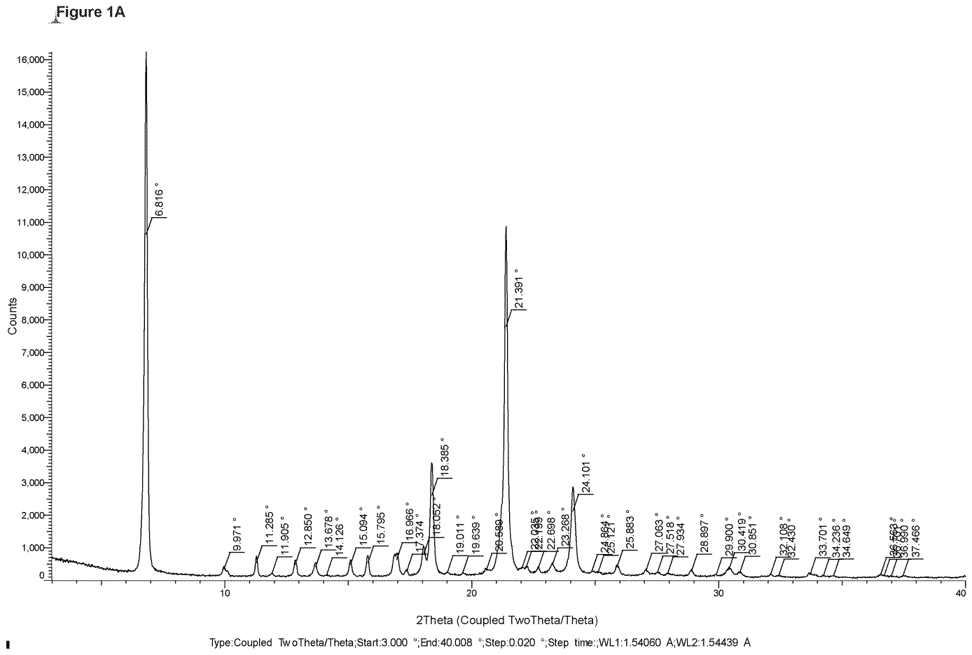

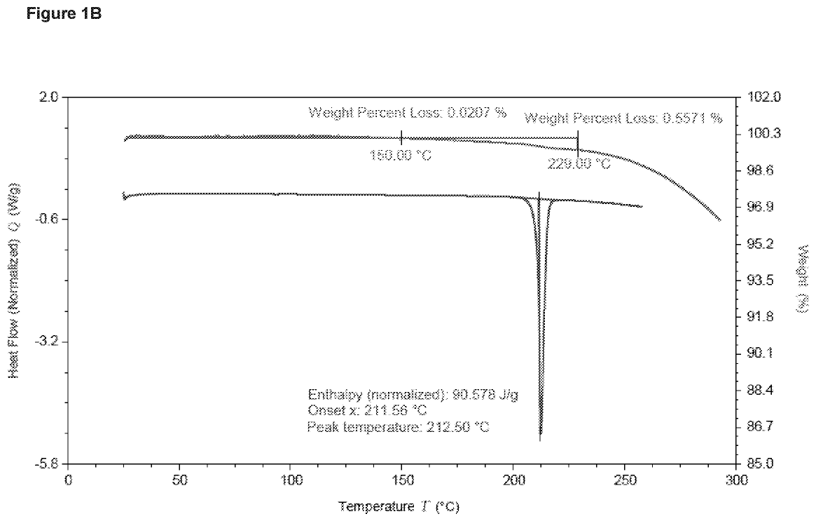

Representative XRPD peaks are tabulated in Table 1. The XRPD patterns and peaks are shown in FIG. 1A. In yet another embodiment, Form II is characterized by an X-ray powder diffraction pattern substantially similar to FIG. 1A. The Thermogravimetric Analysis (TGA) and Differential Scanning Calorimetry Analysis (DSC) thermograms are shown in FIG. 1B.

In one embodiment, crystalline Form I of Compound (I) is an anhydrate.

TABLE 1
Representative XRPD peaks of crystalline Form I of Compound (I)

| Peak | Angle | Rel. Intensity |
|---|---|---|
| 1 | 6.816° | 100.0% |
| 2 | 9.971° | 2.0% |
| 3 | 11.285° | 4.1% |
| 4 | 11.905° | 0.7% |
| 5 | 12.850° | 3.4% |
| 6 | 13.678° | 2.7% |
| 7 | 14.126° | 0.4% |
| 8 | 15.094° | 3.4% |
| 9 | 15.795° | 3.9% |
| 10 | 16.966° | 4.8% |
| 11 | 17.374° | 1.2% |
| 12 | 18.052° | 6.1% |
| 13 | 18.385° | 23.5% |
| 14 | 19.011° | 0.6% |
| 15 | 19.639° | 0.6% |
| 16 | 20.589° | 1.0% |
| 17 | 21.391° | 72.9% |
| 18 | 22.035° | 1.2% |
| 19 | 22.199° | 1.3% |
| 20 | 22.698° | 1.2% |
| 21 | 23.268° | 2.3% |
| 22 | 24.101° | 18.2% |
| 23 | 24.864° | 0.5% |
| 24 | 25.121° | 0.4% |
| 25 | 25.883° | 2.2% |
| 26 | 27.063° | 1.5% |
| 27 | 27.518° | 0.7% |
| 28 | 27.934° | 0.4% |
| 29 | 28.897° | 1.4% |
| 30 | 29.900° | 0.5% |
| 31 | 30.419° | 2.0% |
| 32 | 30.851° | 1.0% |
| 33 | 32.108° | 0.8% |
| 34 | 32.430° | 0.4% |
| 35 | 33.701° | 0.7% |
| 36 | 34.236° | 0.3% |

### Form II

In another aspect, the present disclosure provides a solid form of Compound (I),

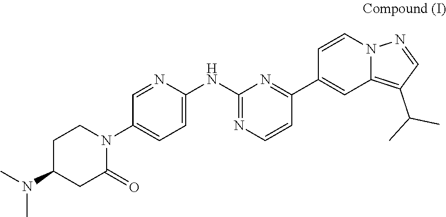 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form II.

In one embodiment the present disclosure provides crystalline Form II of Compound (I), which is characterized by a XRPD diffraction pattern comprising a XRPD peak at an angle of diffraction at about 4.7° (±0.2), 5.0° (±0.2), 8.4° (±0.2), 9.0° (±0.2), 11.5° (±0.2), 13.8° (±0.2), 19.5° (±0.2), 21.8° (±0.2), 23.2° (±0.2), or 23.5° (±0.2) in 2θ.

In another embodiment, the crystalline Form II of Compound (I) is characterized by a XRPD diffraction pattern comprising at least two XRPD peaks selected from XRPD peaks at an angle of diffraction at about 4.7° (±0.2), 5.0° (±0.2), 8.4° (±0.2), and 21.8° (±0.2) in 2θ. In another embodiment, the crystalline Form II of Compound (I) is characterized by a XRPD diffraction pattern comprising XRPD peaks at an angle of diffraction at about 4.7° (±0.2) and 5.0° (±0.2) in 2θ. In another embodiment, the crystalline Form II of Compound (I) is characterized by a XRPD diffraction pattern comprising XRPD peaks at an angle of diffraction at about 4.7° (±0.2) and 8.4° (±0.2) in 2θ. In another embodiment, the crystalline Form II of Compound (I) is characterized by a XRPD diffraction pattern comprising XRPD peaks at an angle of diffraction at about 4.7° (±0.2) and 21.8° (±0.2) in 2θ. In another embodiment, the crystalline Form II of Compound (I) is characterized by a XRPD diffraction pattern comprising XRPD peaks at an angle of diffraction at about 5.0° (±0.2) and 8.4° (±0.2) in 2θ. In yet another embodiment, the crystalline Form II of Compound (I) is characterized by a XRPD diffraction pattern comprising XRPD peaks at an angle of diffraction at about 5.0° (±0.2) and 21.8° (±0.2) in 2θ. In yet another embodiment, the crystalline Form II of Compound (I) is characterized by a XRPD diffraction pattern comprising XRPD peaks at an angle of diffraction at about 8.4° (±0.2) and 21.8° (±0.2) in 2θ.

In yet another embodiment, the crystalline Form II of Compound (I) is characterized by a XRPD diffraction pattern comprising at least three XRPD peaks selected from XRPD peaks at an angle of diffraction at about 4.7° (±0.2), 5.0° (±0.2), 8.4° (±0.2), and 21.8° (±0.2) in 2θ. In yet another embodiment, the crystalline Form II of Compound (I) is characterized by a XRPD diffraction pattern comprising XRPD peaks at an angle of diffraction at about 4.7° (±0.2), 5.0° (±0.2), and 8.4° (±0.2) in 2θ. In yet another embodiment, the crystalline Form II of Compound (I) is characterized by a XRPD diffraction pattern comprising XRPD peaks at an angle of diffraction at about 4.7° (±0.2), 5.0° (±0.2), and 21.8° (±0.2) in 2θ. In yet another embodiment, the crystalline Form II of Compound (I) is characterized by a XRPD diffraction pattern comprising XRPD peaks at an angle of diffraction at about 4.7° (±0.2), 8.4° (±0.2), and 21.8° (±0.2) in 2θ. In yet another embodiment, the crystalline Form II of Compound (I) is characterized by a XRPD diffraction pattern comprising XRPD peaks at an angle of diffraction at about 5.0° (±0.2), 8.4° (±0.2), and 21.8° (±0.2) in 2θ.

In yet another embodiment, the crystalline Form II of Compound (I) is characterized by a XRPD diffraction pattern further comprising one or more XRPD peaks at about 13.8° (±0.2), 19.5° (±0.2), and 23.2° (±0.2) in 2θ. In yet another embodiment, the crystalline Form II of Compound (I) is characterized by a XRPD diffraction pattern further comprising peaks at about 13.8° (±0.2), 19.5° (±0.2), and 23.2° (±0.2) in 2θ.

In yet another embodiment, the crystalline Form II of Compound (I) is characterized by a XRPD diffraction pattern comprising at least four XRPD peaks selected from XRPD peaks at about 4.7° (±0.2), 5.0° (±0.2), 8.4° (±0.2), 13.8° (±0.2), 19.5° (±0.2), 21.8° (±0.2), and 23.2° (±0.2) in 2θ. In yet another embodiment, the crystalline Form II of Compound (I) is characterized by a XRPD diffraction pattern comprising at least five XRPD peaks selected from XRPD peaks at about 4.7° (±0.2), 5.0° (±0.2), 8.4° (±0.2), 13.8° (±0.2), 19.5° (±0.2), 21.8° (±0.2), and 23.2° (±0.2) in 2θ.

In yet another embodiment, the crystalline Form II of Compound (I) is characterized by a XRPD diffraction pattern comprising a XRPD peak at an angle of diffraction at about 4.7° (±0.2) in 2θ, and at least two additional XRPD peaks selected from XRPD peaks at 5.0° (±0.2), 8.4° (±0.2), 13.8° (±0.2), 19.5° (±0.2), 21.8° (±0.2), and 23.2° (±0.2) in 2θ. In yet another embodiment, the crystalline Form II of Compound (I) is characterized by a XRPD diffraction pattern comprising a XRPD peak at an angle of diffraction at about 4.7° (±0.2) in 2θ, and at least three additional XRPD peaks selected from XRPD peaks at 5.0° (±0.2), 8.4° (±0.2), 13.8° (±0.2), 19.5° (±0.2), 21.8° (±0.2), and 23.2° (±0.2) in 2θ.

In yet another embodiment, the crystalline Form II of Compound (I) is characterized by a XRPD diffraction pattern comprising a XRPD peak at an angle of diffraction at about 5.0° (±0.2) in 2θ, and at least two additional XRPD peaks selected from XRPD peaks at 4.7° (±0.2), 8.4° (±0.2), 13.8° (±0.2), 19.5° (±0.2), 21.8° (±0.2), and 23.2° (±0.2) in 2θ. In yet another embodiment, the crystalline Form II of Compound (I) is characterized by a XRPD diffraction pattern comprising a XRPD peak at an angle of diffraction at about 5.0° (±0.2) in 2θ, and at least three additional XRPD peaks selected from XRPD peaks at 4.7° (±0.2), 8.4° (±0.2), 13.8° (±0.2), 19.5° (±0.2), 21.8° (±0.2), and 23.2° (±0.2) in 2θ.

In yet another embodiment, the crystalline Form II of Compound (I) is characterized by a XRPD diffraction pattern comprising a XRPD peak at an angle of diffraction at about 21.8° (±0.2) in 2θ, and at least two additional XRPD peaks selected from XRPD peaks at 4.7° (±0.2), 5.0° (±0.2), 8.4° (±0.2), 13.8° (±0.2), 19.5° (±0.2), and 23.2° (±0.2) in 2θ. In yet another embodiment, the crystalline Form II of Compound (I) is characterized by a XRPD diffraction pattern comprising a XRPD peak at an angle of diffraction at about 4.7° (±0.2) in 2θ, and at least three additional XRPD peaks selected from XRPD peaks at 4.7° (±0.2), 5.0° (±0.2), 8.4° (±0.2), 13.8° (±0.2), 19.5° (±0.2), and 23.2° (±0.2) in 2θ.

In yet another embodiment, the crystalline Form II of Compound (I) is characterized by a XRPD diffraction pattern further comprising one or more XRPD peaks at about 9.0° (±0.2), 11.5° (±0.2), and 23.5° (±0.2) in 2θ. In yet another embodiment, the crystalline Form II of Compound (I) is characterized by a XRPD diffraction pattern further comprising peaks at about 9.0° (±0.2), 11.5° (±0.2), and 23.5° (±0.2) in 2θ.

In yet another embodiment, the crystalline Form II of Compound (I) is characterized by a XRPD diffraction pattern comprising XRPD peaks at an angle of diffraction at about 4.7° (±0.2) and 5.0° (±0.2) in 2θ, and at least one additional XRPD peaks selected from XRPD peaks at 8.4° (±0.2), 9.0° (±0.2), 11.5° (±0.2), 13.8° (±0.2), 19.5° (±0.2), 21.8° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 4.7° (±0.2) and 5.0° (±0.2) in 2θ, and at least two additional XRPD peaks selected from XRPD peaks at 8.4° (±0.2), 9.0° (±0.2), 11.5° (±0.2), 13.8° (±0.2), 19.5° (±0.2), 21.8° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 4.7° (±0.2) and 5.0° (±0.2) in 2θ, and at least three additional XRPD peaks selected from XRPD peaks at 8.4° (±0.2), 9.0° (±0.2), 11.5° (±0.2), 13.8° (±0.2), 19.5° (±0.2), 21.8° (±0.2), 23.2° (±0.2), and 23.5 (±0.2) in 2θ.

In yet another embodiment, the crystalline Form II of Compound (I) is characterized by a XRPD diffraction pattern comprising XRPD peaks at an angle of diffraction at about 5.0° (±0.2) and 21.8° (±0.2) in 2θ, and at least one additional XRPD peaks selected from XRPD peaks at 4.7° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 5.0° (±0.2) and 21.8° (±0.2) in 2θ, and at least two additional XRPD peaks selected from XRPD peaks at 4.7° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 5.0° (±0.2) and 21.8° (±0.2) in 2θ, and at least three additional XRPD peaks selected from XRPD peaks at 4.7° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ.

In yet another embodiment, the crystalline Form II of Compound (I) is characterized by a XRPD diffraction pattern comprising XRPD peaks at an angle of diffraction at about 4.7° (±0.2) and 21.8° (±0.2) in 2θ, and at least one additional XRPD peaks selected from XRPD peaks at 5.0° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 4.7° (±0.2) and 21.8° (±0.2) in 2θ, and at least two additional XRPD peaks selected from XRPD peaks at 5.0° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ. In yet another embodiment, the crystalline Form II of Compound (I) is characterized by a XRPD diffraction pattern comprising XRPD peaks at an angle of diffraction at about 4.7° (±0.2) and 21.8° (±0.2) in 2θ, and at least three additional XRPD peaks selected from XRPD peaks at 5.0° (±0.2), 8.4° (±0.2), 9.0° (±0.2), 11.5° (±0.2), 13.8° (±0.2), 19.5° (±0.2), 23.2° (±0.2), and 23.5 (±0.2) in 2θ.

In yet another embodiment, the crystalline Form II of Compound (I) is characterized by a XRPD diffraction pattern comprising XRPD peaks at an angle of diffraction at about 4.7° (±0.2), 5.0° (±0.2), 8.4° (±0.2), 9.0° (±0.2), 11.5° (±0.2), 13.8° (±0.2), 19.5° (±0.2), 21.8° (±0.2), 23.2° (±0.2), and 23.5° (±0.2) in 2θ.

In one embodiment, the present disclosure provides crystalline Form II of Compound (I), which is characterized by XRPD peaks at 4.7°, 5.0°, 8.4°, and 21.8°±0.2 in 2θ. In another embodiment, the present disclosure provides a crystalline Form II of Compound (I), which is characterized by XRPD peaks at 427, 5.00, 8.40, 13.8°, 19.50, 21.80, and 23.20±0.2 in 2θ. In another embodiment, the present disclosure provides a crystalline Form II of Compound (I), which is characterized by XRPD peaks at 4.70, 5.0°, 8.40, 9.00, 11.50, 13.80, 19.50, 21.80, 23.20, and 23.5°±0.2 in 2θ.

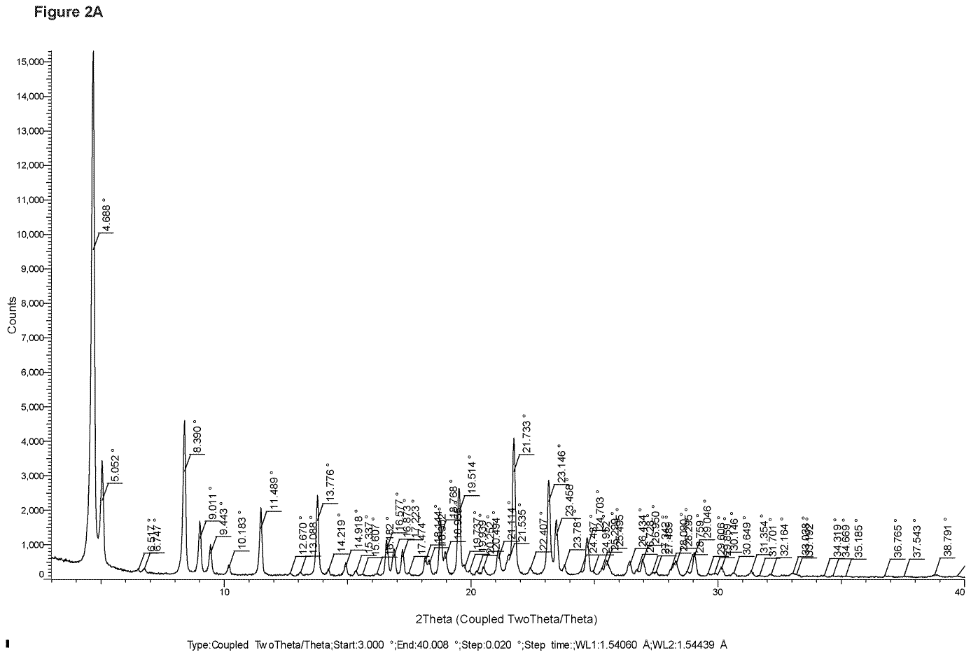

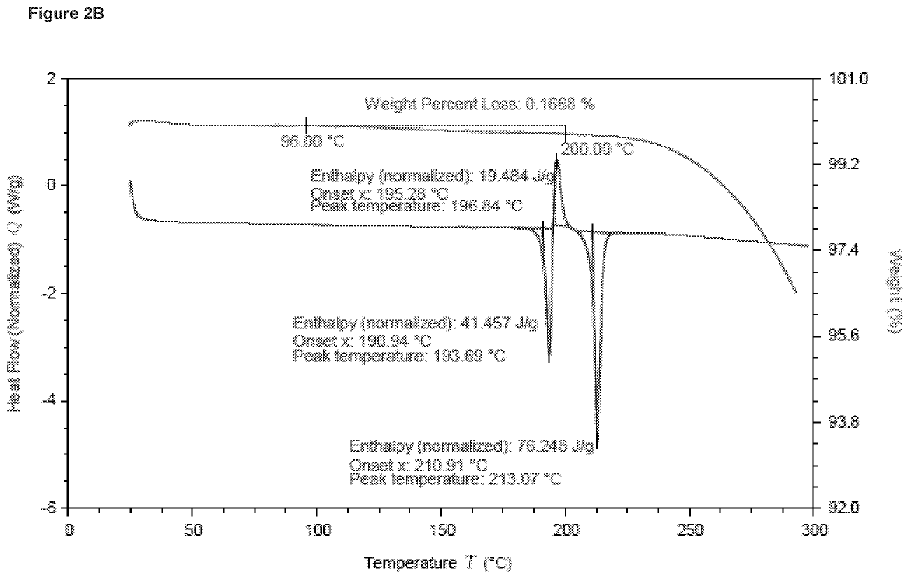

Representative XRPD peaks are tabulated in Table 2. The XRPD patterns and peaks are shown in FIG. 2A. The Thermogravimetric Analysis (TGA) and Differential Scanning Calorimetry Analysis (DSC) thermograms are shown in FIG. 2B. In one embodiment, crystalline Form II of Compound (I) is characterized by differential scanning calorimeter (DSC) peak phase transition temperatures of 190.9±2° C., 195.3±2° C. and 210.9±2° C.

In one embodiment, crystalline Form II of Compound (I) is an anhydrate.

TABLE 2
Representative XRPD peaks of crystalline
Form II of Compound (I)

| Peak | Angle | Rel. Intensity |
|---|---|---|
| 1 | 4.688° | 100% |
| 2 | 5.052° | 20.9% |
| 3 | 6.517° | 0.7% |
| 4 | 6.747° | 0.9% |
| 5 | 8.390° | 32.5% |
| 6 | 9.011° | 11.3% |
| 7 | 9.443° | 6.5% |
| 8 | 10.183° | 2.1% |
| 9 | 11.489° | 14.8% |
| 10 | 12.670° | 0.7% |
| 11 | 13.088° | 0.8% |
| 12 | 13.776° | 17.5% |
| 13 | 14.219° | 1.6% |
| 14 | 14.918° | 2.8% |
| 15 | 15.337° | 1.0% |
| 16 | 15.607° | 0.3% |
| 17 | 16.182° | 0.4% |
| 18 | 16.577° | 8.1% |
| 19 | 16.873° | 6.1% |
| 20 | 17.223° | 5.9% |
| 21 | 17.474° | 0.6% |
| 22 | 18.144° | 4.2% |
| 23 | 18.302° | 3.2% |
| 24 | 18.768° | 12.0% |
| 25 | 18.955° | 5.0% |
| 26 | 19.514° | 19.6% |
| 27 | 19.737° | 2.0% |
| 28 | 19.939° | 1.0% |
| 29 | 20.252° | 0.7% |
| 30 | 20.494° | 2.0% |
| 31 | 21.114° | 5.2% |
| 32 | 21.535° | 4.5% |
| 33 | 21.733° | 32.6% |
| 34 | 22.407° | 1.6% |
| 35 | 23.146° | 23.0% |
| 36 | 23.458° | 12.3% |
| 37 | 23.781° | 2.4% |
| 38 | 24.437° | 0.8% |
| 39 | 24.703° | 8.5% |
| 40 | 24.952° | 0.8% |
| 41 | 25.299° | 1.7% |
| 42 | 25.495° | 3.0% |
| 43 | 26.434° | 3.1% |
| 44 | 26.728° | 1.3% |
| 45 | 26.950° | 4.1% |
| 46 | 27.342° | 0.8% |
| 47 | 27.489° | 0.7% |
| 48 | 28.090° | 1.7% |
| 49 | 28.295° | 3.2% |
| 50 | 28.759° | 1.3% |
| 51 | 29.046° | 6.0% |
| 52 | 29.606° | 0.7% |
| 53 | 29.859° | 0.4% |
| 54 | 30.146° | 2.1% |
| 55 | 30.649° | 0.9% |
| 56 | 31.354° | 1.5% |
| 57 | 31.701° | 0.4% |
| 58 | 32.164° | 0.6% |
| 59 | 33.038° | 0.8% |
| 60 | 33.192° | 0.6% |
| 61 | 34.319° | 0.5% |
| 62 | 34.669° | 0.4% |
| 63 | 35.185° | 0.3% |
| 64 | 36.765° | 0.3% |
| 65 | 37.543° | 0.3% |
| 66 | 38.791° | 0.6% |
| 67 | 39.706° | 0.4% |

### Form III

In another aspect, the present disclosure provides a solid form of Compound (I),

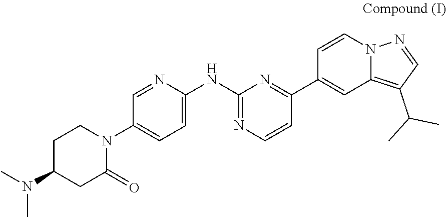 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form III.

In one embodiment the present disclosure provides crystalline Form III of Compound (I), which is characterized by a XRPD diffraction pattern comprising a XRPD peak at an angle of diffraction at about 5.7° (±0.2), 7.1° (±0.2), 7.4° (±0.2), 10.5° (±0.2), 11.2° (±0.2), 14.2° (±0.2), 17.0° (±0.2), 20.1° (±0.2), 20.5° (±0.2), or 21.1° (±0.2) in 2θ.

In another embodiment, the crystalline Form III of Compound (I) is characterized by a XRPD diffraction pattern comprising at least two XRPD peaks selected from XRPD peaks at an angle of diffraction at about 5.7° (±0.2), 7.4° (±0.2), 10.5° (±0.2), 14.2° (±0.2), and 20.5° (±0.2) in 2θ. In another embodiment, the crystalline Form III of Compound (I) is characterized by a XRPD diffraction pattern comprising XRPD peaks at an angle of diffraction at about 10.5° (±0.2) and 14.2° (±0.2) in 2θ. In another embodiment, the crystalline Form III of Compound (I) is characterized by a XRPD diffraction pattern comprising XRPD peaks at an angle of diffraction at about 10.5° (±0.2) and 20.5° (±0.2) in 2θ. In another embodiment, the crystalline Form III of Compound (I) is characterized by a XRPD diffraction pattern comprising XRPD peaks at an angle of diffraction at about 10.5° (±0.2) and 5.7° (±0.2) in 2θ. In another embodiment, the crystalline Form III of Compound (I) is characterized by a XRPD diffraction pattern comprising XRPD peaks at an angle of diffraction at about 10.5° (±0.2) and 7.4° (±0.2) in 2θ. In yet another embodiment, the crystalline Form III of Compound (I) is characterized by a XRPD diffraction pattern comprising XRPD peaks at an angle of diffraction at about 14.2° (±0.2) and 20.5° (±0.2) in 2θ. In yet another embodiment, the crystalline Form III of Compound (I) is characterized by a XRPD diffraction pattern comprising XRPD peaks at an angle of diffraction at about 14.2° (±0.2) and 5.7° (±0.2) in 2θ. In yet another embodiment, the crystalline Form III of Compound (I) is characterized by a XRPD diffraction pattern comprising XRPD peaks at an angle of diffraction at about 14.2° (±0.2) and 7.4° (±0.2) in 2θ. In yet another embodiment, the crystalline Form III of Compound (I) is characterized by a XRPD diffraction pattern comprising XRPD peaks at an angle of diffraction at about 20.5° (±0.2) and 5.7° (±0.2) in 2θ. In yet another embodiment, the crystalline Form III of Compound (I) is characterized by a XRPD diffraction pattern comprising XRPD peaks at an angle of diffraction at about 20.5° (±0.2) and 7.4° (±0.2) in 2θ.

In yet another embodiment, the crystalline Form III of Compound (I) is characterized by a XRPD diffraction pattern comprising at least three XRPD peaks selected from XRPD peaks at an angle of diffraction at about 5.7° (±0.2), 7.4° (±0.2), 10.5° (±0.2), 14.2° (±0.2), and 20.5° (±0.2) in 2θ. In yet another embodiment, the crystalline Form III of Compound (I) is characterized by a XRPD diffraction pattern comprising XRPD peaks at an angle of diffraction at about 10.5° (±0.2), 14.2° (±0.2), and 20.5° (±0.2) in 2θ. In yet another embodiment, the crystalline Form III of Compound (I) is characterized by a XRPD diffraction pattern comprising XRPD peaks at an angle of diffraction at about 10.5° (±0.2), 14.2° (±0.2), and 5.7° (±0.2) in 2θ. In yet another embodiment, the crystalline Form III of Compound (I) is characterized by a XRPD diffraction pattern comprising XRPD peaks at an angle of diffraction at about 10.5° (±0.2), 14.2° (±0.2), and 7.4° (±0.2) in 2θ. In yet another embodiment, the crystalline Form III of Compound (I) is characterized by a XRPD diffraction pattern comprising XRPD peaks at an angle of diffraction at about 14.2° (±0.2), 20.5° (±0.2) and 5.7° (±0.2) in 2θ. In yet another embodiment, the crystalline Form III of Compound (I) is characterized by a XRPD diffraction pattern comprising XRPD peaks at an angle of diffraction at about 14.2° (±0.2), 20.5° (±0.2) and 7.4° (±0.2) in 2θ. In yet another embodiment, the crystalline Form III of Compound (I) is characterized by a XRPD diffraction pattern comprising XRPD peaks at an angle of diffraction at about 10.5° (±0.2), 5.7° (±0.2), and 20.5° (±0.2) in 2θ. In yet another embodiment, the crystalline Form III of Compound (I) is characterized by a XRPD diffraction pattern comprising XRPD peaks at an angle of diffraction at about 10.5° (±0.2), 7.4° (±0.2), and 20.5° (±0.2) in 2θ.

In yet another embodiment, the crystalline Form III of Compound (I) is characterized by a XRPD diffraction pattern further comprising one or more XRPD peaks at about 17.0° (±0.2) and 20.1° (±0.2) in 2θ. In yet another embodiment, the crystalline Form III of Compound (I) is characterized by a XRPD diffraction pattern further comprising peaks at about 17.0° (±0.2) and 20.1° (±0.2) in 2θ.

In yet another embodiment, the crystalline Form III of Compound (I) is characterized by a XRPD diffraction pattern comprising XRPD peaks at an angle of diffraction at about 5.7° (±0.2), 7.1° (±0.2), 7.4° (±0.2), 10.5° (±0.2), 11.2° (±0.2), 14.2° (±0.2), 17.0° (±0.2), 20.1° (±0.2), 20.5° (±0.2), and 21.1° (±0.2) in 2θ.

In one embodiment, the present disclosure provides crystalline Form III of Compound (I), which is characterized by XRPD peaks at 5.7°, 7.4°, 10.5°, 14.2°, and 20.5°±0.2 in 2θ. In another embodiment, the present disclosure provides a crystalline Form III of Compound
(I), which is characterized by XRPD peaks at 5.7°, 7.4°, 10.5°, 14.2°, 17.0°, 20.10, and 20.5° ±0.2 in 2θ. In another embodiment, the present disclosure provides a crystalline Form III of Compound (I), which is characterized by XRPD peaks at 5.7°, 7.1°, 7.4°, 10.5°, 11.2°, 14.2°, 17.0°, 20.1°, 20.5°, and 21.1°±0.2 in 2θ.

In another embodiment, the present disclosure provides a crystalline Form III of Compound (I), which is characterized by differential scanning calorimeter (DSC) peak phase transition temperature of 196.4±2° C., 199.6±2° C., and 211.3±2° C.

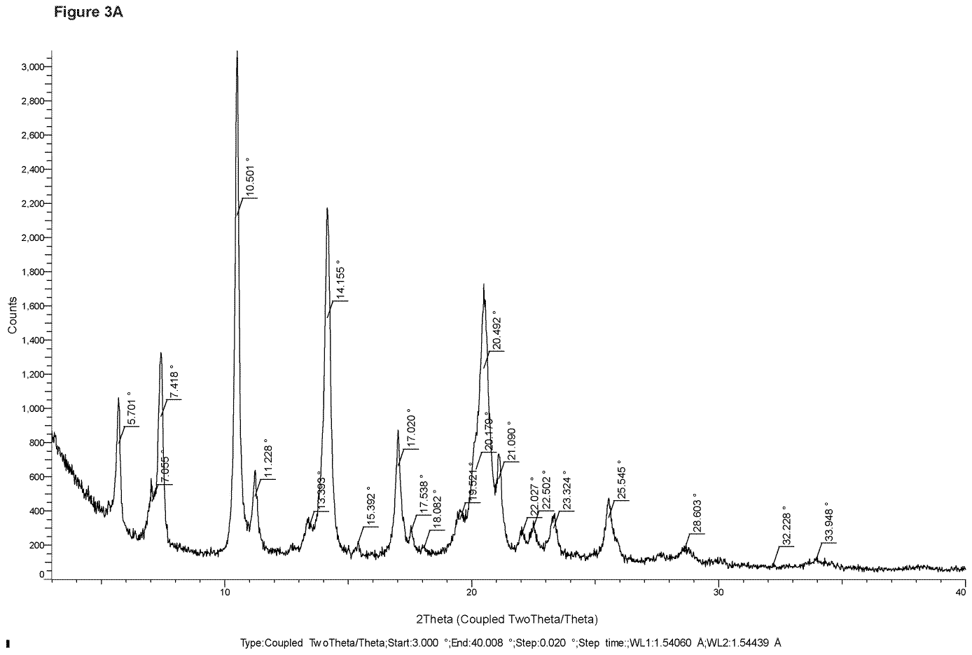

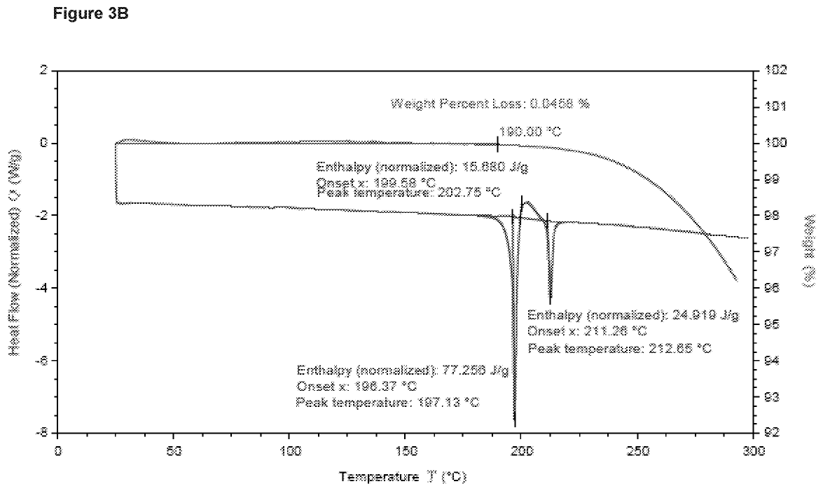

Representative XRPD peaks are tabulated in Table 3. The XRPD patterns and peaks are shown in FIG. 3A. The Thermogravimetric Analysis (TGA) and Differential Scanning Calorimetry Analysis (DSC) thermograms are shown in FIG. 3B.

In one embodiment, crystalline Form III of Compound (I) is an anhydrate.

TABLE 3
Representative XRPD peaks of crystalline
Form III of Compound (I)

| Peak | Angle | Rel. Intensity |
|---|---|---|
| 1 | 5.701° | 24.5% |
| 2 | 7.055° | 11.6% |
| 3 | 7.418° | 37.5% |
| 4 | 10.501° | 100.0% |
| 5 | 11.228° | 16.4% |
| 6 | 13.393° | 7.0% |
| 7 | 14.155° | 69.8% |
| 8 | 15.392° | 3.3% |
| 9 | 17.021° | 26.1% |
| 10 | 17.538° | 6.3% |
| 11 | 18.082° | 1.7% |
| 12 | 19.521° | 9.1% |
| 13 | 20.179° | 23.6% |
| 14 | 20.492° | 53.6% |
| 15 | 21.090° | 20.6% |
| 16 | 22.027° | 4.4% |
| 17 | 22.502° | 6.6% |
| 18 | 23.324° | 7.3% |
| 19 | 25.545° | 12.2% |
| 20 | 28.603° | 3.0% |
| 21 | 32.228° | 1.2% |
| 22 | 33.948° | 2.2% |

### Form IV

In another aspect, the present disclosure provides a solid form of Compound (I),

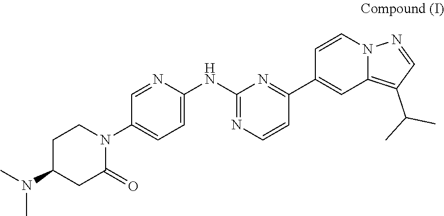 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form IV.

In one embodiment the present disclosure provides crystalline Form IV of Compound (I), which is characterized by a XRPD diffraction pattern comprising a XRPD peak at an angle of diffraction at about 3.8° (±0.2), 5.4° (±0.2), or 15.8° (±0.2) in 2θ.

In another embodiment, the crystalline Form IV of Compound (I) is characterized by a XRPD diffraction pattern comprising at least two XRPD peaks selected from XRPD peaks at an angle of diffraction at about 3.8° (±0.2), 5.4° (±0.2), and 15.8° (±0.2) in 2θ. In another embodiment, the crystalline Form IV of Compound (I) is characterized by a XRPD diffraction pattern comprising XRPD peaks at an angle of diffraction at about 3.8° (±0.2) and 5.4° (±0.2) in 2θ. In another embodiment, the crystalline Form IV of Compound (I) is characterized by a XRPD diffraction pattern comprising XRPD peaks at an angle of diffraction at about 3.8° (±0.2) and 15.8° (±0.2) in 2θ. In another embodiment, the crystalline Form IV of Compound (I) is characterized by a XRPD diffraction pattern comprising XRPD peaks at an angle of diffraction at about 5.4° (±0.2) and 15.8° (±0.2) in 2θ.

In yet another embodiment, the crystalline Form IV of Compound (I) is characterized by a XRPD diffraction pattern comprising XRPD peaks at an angle of diffraction at about 3.8° (±0.2), 5.4° (±0.2), and 15.8° (±0.2) in 2θ.

In one embodiment, the present disclosure provides crystalline Form IV of Compound (I), which is characterized by XRPD peaks at 3.8°, 5.4°, and 15.8°±0.2 in 2θ.

In another embodiment, the present disclosure provides a crystalline Form IV of Compound (I), which is characterized by differential scanning calorimeter (DSC) peak phase transition temperatures of 143.4±2° C., 152.0±2° C., and 211.8±2° C.

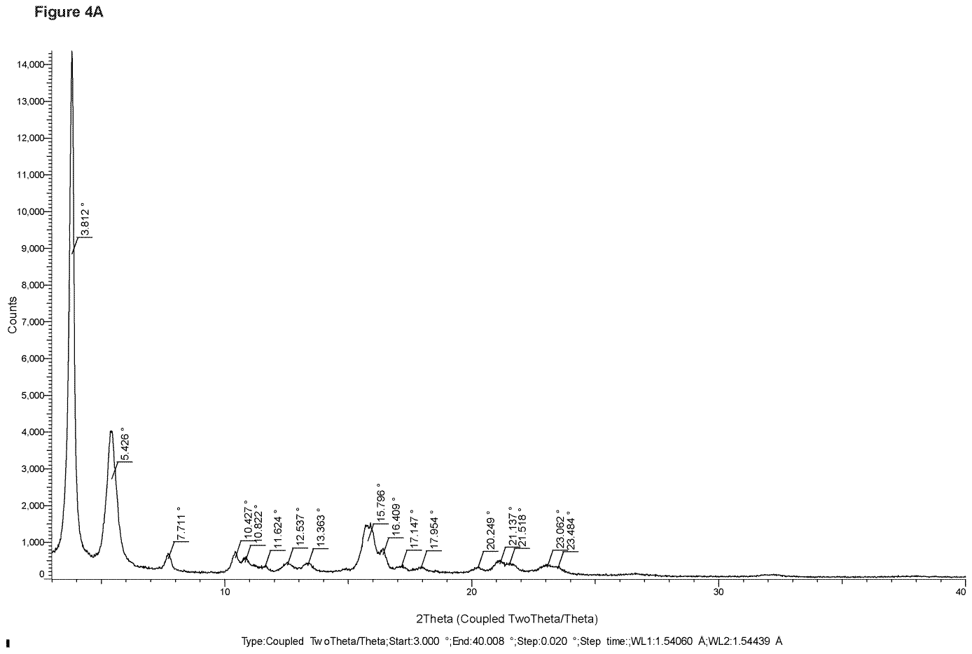

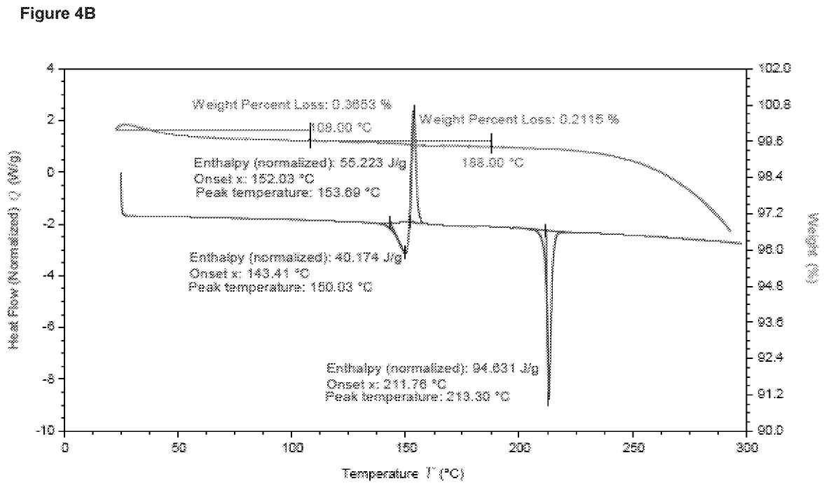

Representative XRPD peaks are tabulated in Table 4. The XRPD patterns and peaks are shown in FIG. 4A. The Thermogravimetric Analysis (TGA) and Differential Scanning Calorimetry Analysis (DSC) thermograms are shown in FIG. 4B.

In one embodiment, crystalline Form IV of Compound (I) is an anhydrate.

TABLE 4
Representative XRPD peaks of crystalline
Form IV of Compound (I)

| Peak | Angle | Rel. Intensity |
|---|---|---|
| 1 | 3.812° | 100.0% |
| 2 | 5.426° | 27.1% |
| 3 | 7.711° | 3.8% |
| 4 | 10.427° | 4.6% |
| 5 | 10.822° | 3.0% |
| 6 | 11.624° | 1.3% |
| 7 | 12.537° | 2.0% |
| 8 | 13.363° | 1.8% |
| 9 | 15.796° | 9.7% |
| 10 | 16.409° | 5.1% |
| 11 | 17.147° | 1.0% |
| 12 | 17.954° | 1.1% |
| 13 | 20.249° | 1.0% |
| 14 | 21.137° | 2.3% |
| 15 | 21.518° | 1.9% |
| 16 | 23.062° | 1.7% |
| 17 | 23.484° | 1.4% |

### Form V

In another aspect, the present disclosure provides a solid form of Compound (I),

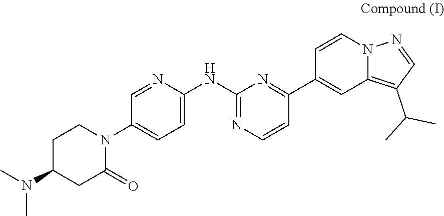 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form V.

In one embodiment the present disclosure provides crystalline Form V of Compound (I), which is characterized by a XRPD diffraction pattern comprising a XRPD peak at an angle of diffraction at about 6.3° (±0.2), 8.8° (±0.2), 11.1° (±0.2), 12.7° (±0.2), 16.0° (±0.2), 18.9° (±0.2), 20.2° (±0.2), 22.2° (±0.2), 23.7° (±0.2), or 27.9 (±0.2) in 2θ.

In another embodiment, the crystalline Form V of Compound (I) is characterized by a XRPD diffraction pattern comprising at least two XRPD peaks selected from XRPD peaks at an angle of diffraction at about 8.8° (±0.2), 11.1° (±0.2), 20.2° (±0.2), and 23.7° (±0.2) in 2θ. In another embodiment, the crystalline Form V of Compound (I) is characterized by a XRPD diffraction pattern comprising XRPD peaks at an angle of diffraction at about 8.8° (±0.2) and 11.1° (±0.2) in 2θ. In another embodiment, the crystalline Form V of Compound (I) is characterized by a XRPD diffraction pattern comprising XRPD peaks at an angle of diffraction at about 8.8° (±0.2) and 20.2° (±0.2) in 2θ. In another embodiment, the crystalline Form V of Compound (I) is characterized by a XRPD diffraction pattern comprising XRPD peaks at an angle of diffraction at about 8.8° (±0.2) and 23.7° (±0.2) in 2θ. In yet another embodiment, the crystalline Form V of Compound (I) is characterized by a XRPD diffraction pattern comprising XRPD peaks at an angle of diffraction at about 11.1° (±0.2) and 20.2° (±0.2) in 2θ. In yet another embodiment, the crystalline Form V of Compound (I) is characterized by a XRPD diffraction pattern comprising XRPD peaks at an angle of diffraction at about 11.1° (±0.2) and 23.7° (±0.2) in 2θ.

In yet another embodiment, the crystalline Form V of Compound (I) is characterized by a XRPD diffraction pattern comprising at least three XRPD peaks selected from XRPD peaks at an angle of diffraction at about 8.8° (±0.2), 11.1° (±0.2), 20.2° (±0.2), and 23.7° (±0.2) in 2θ. In yet another embodiment, the crystalline Form V of Compound (I) is characterized by a XRPD diffraction pattern comprising a XRPD peak at an angle of diffraction at about 8.8° (±0.2) in 2θ, and at least two additional XRPD peaks selected from XRPD peaks at 11.1° (±0.2), 20.2° (±0.2), and 23.7° (±0.2) in 2θ. In yet another embodiment, the crystalline Form V of Compound (I) is characterized by a XRPD diffraction pattern comprising a XRPD peak at an angle of diffraction at about 11.1° (±0.2) in 2θ, and at least two additional XRPD peaks selected from XRPD peaks at 8.8° (±0.2), 20.2° (±0.2), and 23.7° (±0.2) in 2θ.

In yet another embodiment, the crystalline Form V of Compound (I) is characterized by a XRPD diffraction pattern further comprising one or more XRPD peaks at about 6.3° (±0.2), 18.9° (±0.2) and 27.9° (±0.2) in 2θ. In yet another embodiment, the crystalline Form V of Compound (I) is characterized by a XRPD diffraction pattern further comprising peaks at about 6.3° (±0.2), 18.9° (±0.2) and 27.9° (±0.2) in 2θ.

In yet another embodiment, the crystalline Form V of Compound (I) is characterized by a XRPD diffraction pattern comprising XRPD peaks at an angle of diffraction at about 6.3° (±0.2), 8.8° (±0.2), 11.1° (±0.2), 12.7° (±0.2), 16.0° (±0.2), 18.9° (±0.2), 20.2° (±0.2), 22.2° (±0.2), 23.7° (±0.2), and 27.9 (±0.2) in 2θ.

In one embodiment, the present disclosure provides crystalline Form V of Compound (I), which is characterized by XRPD peaks at 8.8°, 11.1°, 20.2°, and 23.7°±0.2 in 2θ. In another embodiment, the present disclosure provides a crystalline Form V of Compound (I), which is characterized by XRPD peaks at 6.3°, 8.8°, 11.1°, 18.9°, 20.2°, 23.7°, and 27.9°±0.2 in 2θ. In another embodiment, the present disclosure provides a crystalline Form V of Compound (I), which is characterized by XRPD peaks at 6.3°, 8.8°, 11.1°, 12.7°, 16.0°, 18.9°, 20.2°, 22.2°, 23.7°, and 27.9°±0.2 in 2θ.

In another embodiment, the present disclosure provides a crystalline Form V of Compound (I), which is characterized by differential scanning calorimeter (DSC) peak phase transition temperature of 100.3±2° C., 187.4±2° C., 195.2±2° C., and 211.8±2° C.

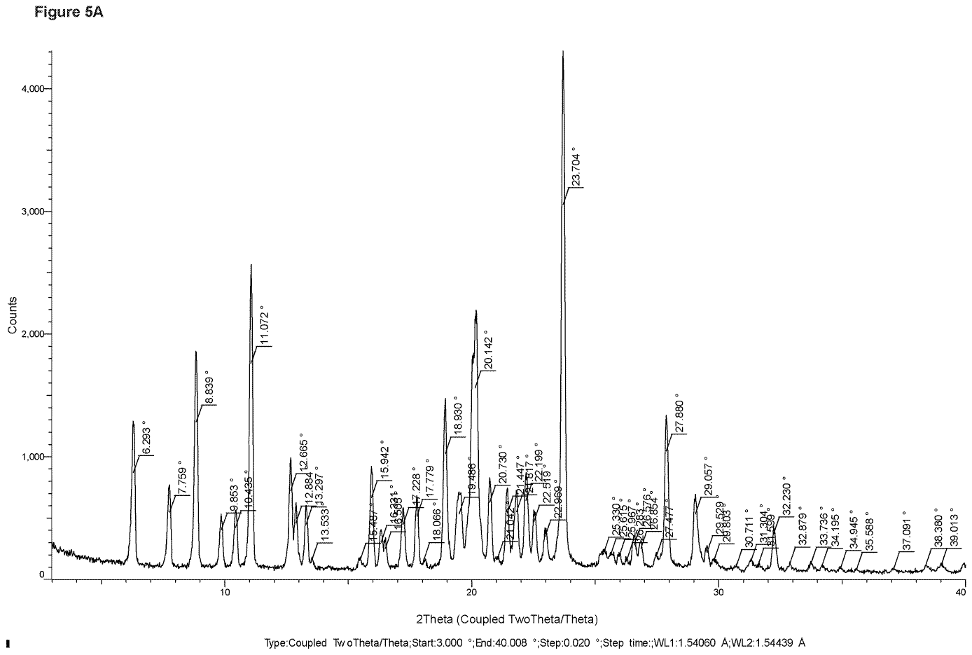

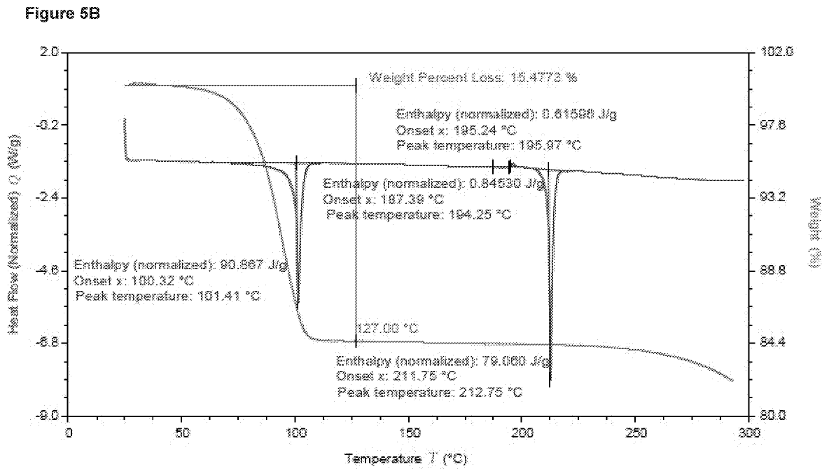

Representative XRPD peaks are tabulated in Table 5. The XRPD patterns and peaks are shown in FIG. 5A. The Thermogravimetric Analysis (TGA) and Differential Scanning Calorimetry Analysis (DSC) thermograms are shown in FIG. 5B.

In one embodiment, crystalline Form V of Compound (I) is a solvate of toluene (1 mol).

TABLE 5
Representative XRPD peaks of crystalline
Form V of Compound (I)

| Peak | Angle | Rel. Intensity |
|---|---|---|
| 1 | 6.293° | 25.5% |
| 2 | 7.759° | 15.0% |
| 3 | 8.839° | 40.3% |
| 4 | 9.853° | 10.2% |
| 5 | 10.435° | 10.7% |
| 6 | 11.072° | 56.6% |
| 7 | 12.665° | 21.4% |
| 8 | 12.884° | 12.4% |
| 9 | 13.297° | 12.1% |
| 10 | 13.533° | 2.2% |
| 11 | 15.487° | 2.3% |
| 12 | 15.942° | 19.6% |
| 13 | 16.331° | 6.9% |
| 14 | 16.505° | 5.1% |
| 15 | 17.228° | 11.7% |
| 16 | 17.779° | 14.0% |
| 17 | 18.066° | 2.0% |
| 18 | 18.930° | 31.0% |
| 19 | 19.486° | 13.9% |
| 20 | 20.142° | 48.8% |
| 21 | 20.730° | 16.8% |
| 22 | 21.042° | 1.2% |
| 23 | 21.447° | 13.7% |
| 24 | 21.817° | 13.9% |
| 25 | 22.199° | 17.1% |
| 26 | 22.519° | 11.1% |
| 27 | 22.969° | 7.0% |
| 28 | 23.704° | 100.0% |
| 29 | 25.330° | 3.0% |
| 30 | 25.615° | 2.6% |
| 31 | 25.967° | 2.3% |
| 32 | 26.283° | 1.5% |
| 33 | 26.576° | 5.6% |
| 34 | 26.854° | 4.9% |
| 35 | 27.477° | 2.8% |
| 36 | 27.880° | 32.1% |
| 37 | 29.057° | 14.7% |
| 38 | 29.529° | 4.1% |
| 39 | 29.803° | 1.9% |
| 40 | 30.711° | 1.0% |
| 41 | 31.304° | 2.6% |
| 42 | 31.599° | 1.5% |
| 43 | 32.230° | 10.2% |
| 44 | 32.879° | 1.9% |
| 45 | 33.736° | 1.9% |
| 46 | 34.195° | 1.6% |
| 47 | 34.945° | 1.2% |
| 48 | 35.588° | 0.8% |
| 49 | 37.091° | 1.0% |
| 50 | 38.380° | 1.5% |
| 51 | 39.013° | 1.8% |

### Form VI

In another aspect, the present disclosure provides a solid form of Compound (I),

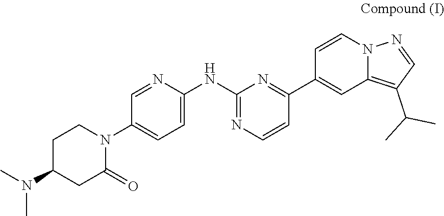 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form VI.

In one embodiment the present disclosure provides crystalline Form VI of Compound (I), which is characterized by a XRPD diffraction pattern comprising a XRPD peak at an angle of diffraction at about 5.6° (±0.2), 9.9° (±0.2), 13.7° (±0.2), or 19.3° (±0.2) in 2θ.

In another embodiment, the crystalline Form VI of Compound (I) is characterized by a XRPD diffraction pattern comprising at least two XRPD peaks selected from XRPD peaks at an angle of diffraction at about 5.6° (±0.2), 9.9° (±0.2), 13.7° (±0.2), and 19.3° (±0.2) in 2θ. In another embodiment, the crystalline Form VI of Compound (I) is characterized by a XRPD diffraction pattern comprising XRPD peaks at an angle of diffraction at about 9.9° (±0.2) and 5.6° (±0.2) in 2θ. In another embodiment, the crystalline Form VI of Compound (I) is characterized by a XRPD diffraction pattern comprising XRPD peaks at an angle of diffraction at about 9.9° (±0.2) and 13.7° (±0.2) in 2θ. In another embodiment, the crystalline Form VI of Compound (I) is characterized by a XRPD diffraction pattern comprising XRPD peaks at an angle of diffraction at about 9.9° (±0.2) and 19.3° (±0.2) in 2θ. In yet another embodiment, the crystalline Form VI of Compound (I) is characterized by a XRPD diffraction pattern comprising XRPD peaks at an angle of diffraction at about 13.7° (±0.2) and 5.6° (±0.2) in 2θ. In yet another embodiment, the crystalline Form VI of Compound (I) is characterized by a XRPD diffraction pattern comprising XRPD peaks at an angle of diffraction at about 13.7° (±0.2) and 19.3° (±0.2) in 2θ.

In yet another embodiment, the crystalline Form VI of Compound (I) is characterized by a XRPD diffraction pattern comprising XRPD peaks at an angle of diffraction at about 5.6° (±0.2), 9.9° (±0.2), 13.7° (±0.2), and 19.3° (±0.2) in 2θ.

In one embodiment, the present disclosure provides crystalline Form VI of Compound (I), which is characterized by XRPD peaks at 5.6°, 9.9°, 13.7°, and 19.3°±0.2 in 2θ.

In another embodiment, the present disclosure provides a crystalline Form VI of Compound (I), which is characterized by differential scanning calorimeter (DSC) peak phase transition temperatures of 43.7±2° C., 185.1±2° C., 194.3±2° C., and 210.2±2° C.

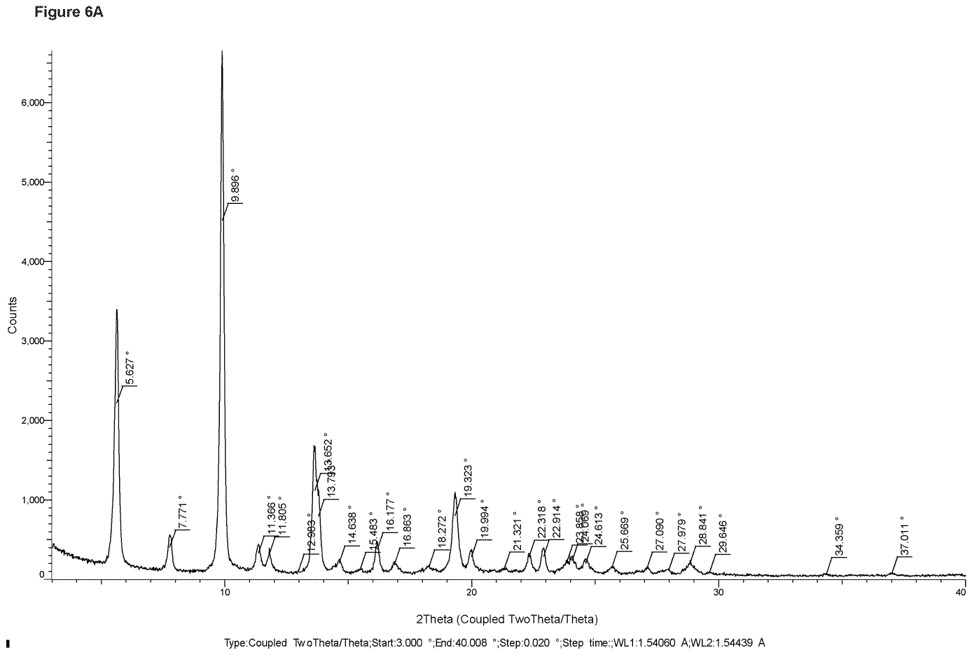

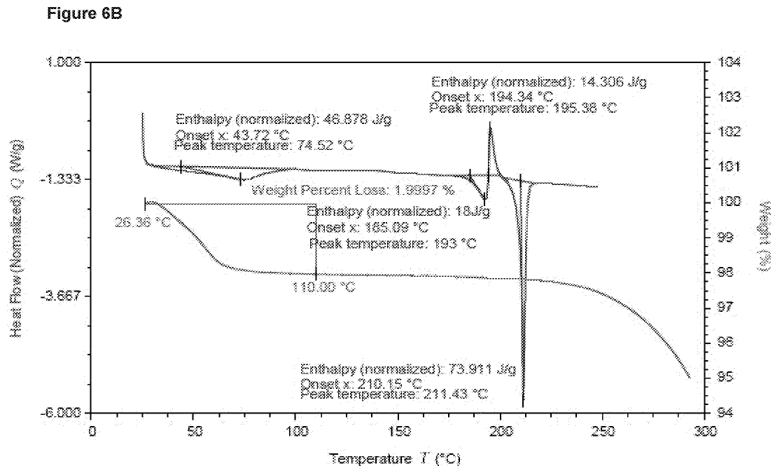

Representative XRPD peaks are tabulated in Table 6. The XRPD patterns and peaks are shown in FIG. 6A. The Thermogravimetric Analysis (TGA) and Differential Scanning Calorimetry Analysis (DSC) thermograms are shown in FIG. 6B.

In one embodiment, crystalline Form VI of Compound (I) is a hemi-hydrate.

TABLE 6
Representative XRPD peaks of crystalline
Form VI of Compound (I)

| Peak | Angle | Rel. Intensity |
|---|---|---|
| 1 | 5.627° | 46.2% |
| 2 | 7.771° | 6.7% |
| 3 | 9.896° | 100.0% |
| 4 | 11.366° | 5.0% |
| 5 | 11.805° | 4.9% |
| 6 | 12.983° | 0.6% |
| 7 | 13.652° | 23.4% |
| 8 | 13.793° | 15.9% |
| 9 | 14.638° | 2.6% |
| 10 | 15.483° | 0.7% |
| 11 | 16.177° | 6.4% |
| 12 | 16.863° | 2.0% |
| 13 | 18.272° | 1.5% |
| 14 | 19.323° | 15.7% |
| 15 | 19.994° | 4.0% |
| 16 | 21.321° | 0.7% |
| 17 | 22.318° | 4.0% |
| 18 | 22.914° | 4.2% |
| 19 | 23.858° | 2.7% |
| 20 | 24.069° | 3.0% |
| 21 | 24.613° | 2.4% |
| 22 | 25.669° | 1.5% |
| 23 | 27.090° | 1.3% |
| 24 | 27.979° | 0.9% |
| 25 | 28.841° | 2.9% |
| 26 | 29.646° | 0.8% |
| 27 | 34.359° | 0.4% |
| 28 | 37.011° | 0.5% |

### Form VII

In another aspect, the present disclosure provides a solid form of Compound (I),

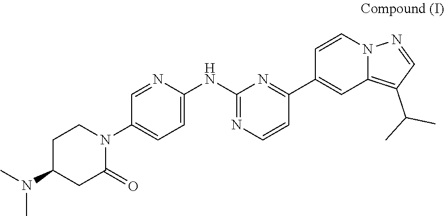 [SMILES: CC(C)c1cnn2ccc(-c3ccnc(Nc4ccc(N5CC[C@H](N(C)C)CC5=O)cn4)n3)cc12]

wherein the solid form is crystalline polymorphic Form VII.

In one embodiment the present disclosure provides crystalline Form VII of Compound (I), which is characterized by a XRPD diffraction pattern comprising a XRPD peak at an angle of diffraction at about 5.3° (±0.2), 5.6° (±0.2), 7.6° (±0.2), 8.2° (±0.2), 10.2° (±0.2), 15.2° (±0.2), 19.3° (±0.2), 20.6° (±0.2), and 23.9° (±0.2) in 2θ.

In another embodiment, the crystalline Form VII of Compound (I) is characterized by a XRPD diffraction pattern comprising at least two XRPD peaks selected from XRPD peaks at an angle of diffraction at about 5.3° (±0.2), 5.6° (±0.2), and 10.2° (±0.2) in 2θ. In another embodiment, the crystalline Form VII of Compound (I) is characterized by a XRPD diffraction pattern comprising XRPD peaks at an angle of diffraction at about 5.3° (±0.2) and 5.6° (±0.2) in 2θ. In another embodiment, the crystalline Form VII of Compound (I) is characterized by a XRPD diffraction pattern comprising XRPD peaks at an angle of diffraction at about 5.3° (±0.2) and 10.2° (±0.2) in 2θ. In another embodiment, the crystalline Form VII of Compound (I) is characterized by a XRPD diffraction pattern comprising XRPD peaks at an angle of diffraction at about 5.6° (±0.2) and 10.2° (±0.2) in 2θ.

In yet another embodiment, the crystalline Form VII of Compound (I) is characterized by a XRPD diffraction pattern further comprising one or more XRPD peaks at about 8.2° (±0.2), 19.3° (±0.2), and 23.9° (±0.2) in 2θ. In yet another embodiment, the crystalline Form VII of Compound (I) is characterized by a XRPD diffraction pattern further comprising peaks at about 8.2° (±0.2), 19.3° (±0.2), and 23.9° (±0.2) in 2θ.

In yet another embodiment, the crystalline Form VII of Compound (I) is characterized by a XRPD diffraction pattern comprising at least three XRPD peaks selected from XRPD peaks at an angle of diffraction at about 5.3° (±0.2), 5.6° (±0.2), 8.2° (±0.2), 10.2° (±0.2), 19.3° (±0.2), and 23.9° (±0.2) in 2θ. In yet another embodiment, the crystalline Form VII of Compound (I) is characterized by a XRPD diffraction pattern comprising XRPD peaks at an angle of diffraction at about 5.3° (±0.2), 10.2° (±0.2), and 5.6° (±0.2) in 2θ. In yet another embodiment, the crystalline Form VII of Compound (I) is characterized by a XRPD diffraction pattern comprising XRPD peaks at an angle of diffraction at about 5.3° (±0.2), 10.2° (±0.2), and 8.2° (±0.2) in 2θ. In yet another embodiment, the crystalline Form VII of Compound (I) is characterized by a XRPD diffraction pattern comprising XRPD peaks at an angle of diffraction at about 5.3° (±0.2), 10.2° (±0.2), and 19.3° (±0.2) in 2θ. In yet another embodiment, the crystalline Form VII of Compound (I) is characterized by a XRPD diffraction pattern comprising XRPD peaks at an angle of diffraction at about 5.3° (±0.2), 10.2° (±0.2), and 23.9° (±0.2) in 2θ.

In yet another embodiment, the crystalline Form VII of Compound (I) is characterized by a XRPD diffraction pattern comprising a XRPD peak at an angle of diffraction at about 5.3° (±0.2) in 2θ, and at least two additional XRPD peaks selected from XRPD peaks at 5.6° (±0.2), 8.2° (±0.2), 10.2° (±0.2), 19.3° (±0.2), and 23.9° (±0.2) in 2θ. In yet another embodiment, the crystalline Form VII of Compound (I) is characterized by a XRPD diffraction pattern comprising a XRPD peak at an angle of diffraction at about 5.3° (±0.2) in 2θ, and at least three additional XRPD peaks selected from XRPD peaks at 5.6° (±0.2), 8.2° (±0.2), 10.2° (±0.2), 19.3° (±0.2), and 23.9° (±0.2) in 2θ.

In yet another embodiment, the crystalline Form VII of Compound (I) is characterized by a XRPD diffraction pattern comprising a XRPD peak at an angle of diffraction at about 10.2° (±0.2) in 2θ, and at least two additional XRPD peaks selected from XRPD peaks at 5.3° (±0.2), 5.6° (±0.2), 8.2° (±0.2), 19.3° (±0.2), and 23.9° (±0.2). In yet another embodiment, the crystalline Form VII of Compound (I) is characterized by a XRPD diffraction pattern comprising a XRPD peak at an angle of diffraction at about 10.2° (±0.2) in 2θ, and at least three additional XRPD peaks selected from XRPD peaks at 5.3° (±0.2), 5.6° (±0.2), 8.2° (±0.2), 19.3° (±0.2), and 23.9° (±0.2) in 2θ.

In yet another embodiment, the crystalline Form VII of Compound (I) is characterized by a XRPD diffraction pattern comprising XRPD peaks at an angle of diffraction at about 5.3° (±0.2), 5.6° (±0.2), 8.2° (±0.2), 19.3° (±0.2), and 23.9° (±0.2) in 2θ.

In yet another embodiment, the crystalline Form VII of Compound (I) is characterized by a XRPD diffraction pattern further comprising one or more XRPD peaks at about 7.6° (±0.2), 15.2° (±0.2), and 20.6° (±0.2) in 2θ. In yet another embodiment, the crystalline Form VII of Compound (I) is characterized by a XRPD diffraction pattern further comprising peaks at about 7.6° (±0.2), 15.2° (±0.2), and 20.6° (±0.2) in 2θ.

In yet another embodiment, the crystalline Form VII of Compound (I) is characterized by a XRPD diffraction pattern comprising XRPD peaks at an angle of diffraction at about 5.3° (±0.2) and 10.2° (±0.2) in 2θ, and at least one additional XRPD peaks selected from XRPD peaks at 5.6° (±0.2), 7.6° (±0.2), 8.2° (±0.2), 15.2° (±0.2), 19.3° (±0.2), 20.6° (±0.2), and 23.9° (±0.2) in 2θ. In yet another embodiment, the crystalline Form VII of Compound (I) is characterized by a XRPD diffraction pattern comprising XRPD peaks at an angle of diffraction at about 5.3° (±0.2) and 10.2° (±0.2) in 2θ, and at least two additional XRPD peaks selected from XRPD peaks at 5.6° (±0.2), 7.6° (±0.2), 8.2° (±0.2), 15.2° (±0.2), 19.3° (±0.2), 20.6° (±0.2), and 23.9° (±0.2) in 2θ. In yet another embodiment, the crystalline Form VII of Compound (I) is characterized by a XRPD diffraction pattern comprising XRPD peaks at an angle of diffraction at about 5.3° (±0.2) and 10.2° (±0.2) in 2θ, and at least three additional XRPD peaks selected from XRPD peaks at 85.6° (±0.2), 7.6° (±0.2), 8.2° (±0.2), 15.2° (±0.2), 19.3° (±0.2), 20.6° (±0.2), and 23.9° (±0.2) in 2θ.

In yet another embodiment, the crystalline Form VII of Compound (I) is characterized by a XRPD diffraction pattern comprising XRPD peaks at an angle of diffraction at about 5.3° (±0.2), 5.6° (±0.2), 7.6° (±0.2), 8.2° (±0.2), 10.2° (±0.2), 15.2° (±0.2), 19.3° (±0.2), 20.6° (±0.2), and 23.9° (±0.2) in 2θ.

In one embodiment, the present disclosure provides crystalline Form VII of Compound (I), which is characterized by XRPD peaks at 5.3 °, 5.6°, and 10.2°±0.2 in 2θ. In another embodiment, the present disclosure provides a crystalline Form VII of Compound (I), which is characterized by XRPD peaks at 5.3°, 5.6°, 8.2°, 10.2°, 19.3°, and 23.9°±0.2 in 2θ.

In another embodiment, the present disclosure provides a crystalline Form VII of Compound (I), which is characterized by XRPD peaks at 5.3°, 5.6°, 7.6°, 8.2°, 10.2°, 15.2°, 19.3°, 20.6°, and 23.9°±0.2 in 2θ.

In another embodiment, the present disclosure provides a crystalline Form VII of Compound (I), which is characterized by differential scanning calorimeter (DSC) peak phase transition temperatures of 184.3±2° C., 192.0±2° C., and 207.8±2° C.

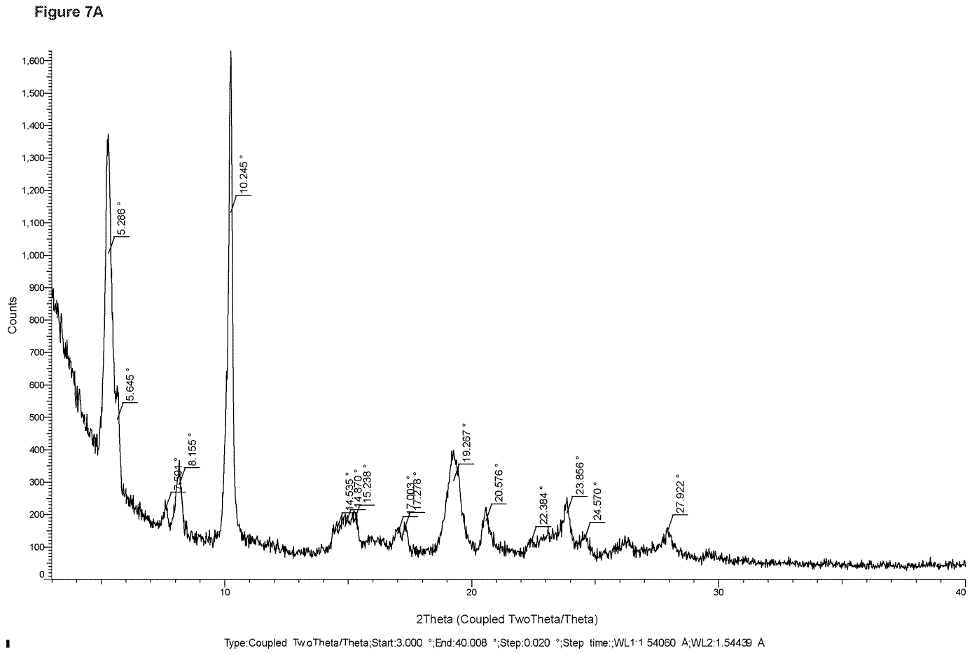

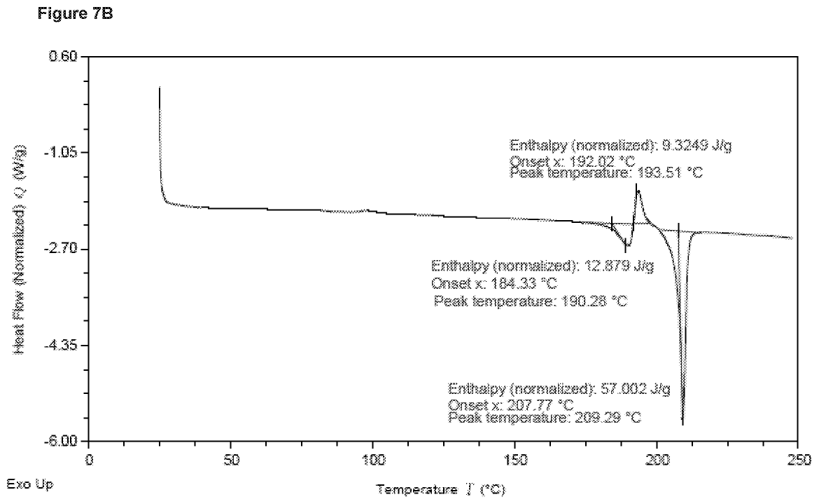

Representative XRPD peaks are tabulated in Table 7. The XRPD patterns and peaks are shown in FIG. 7A. The Thermogravimetric Analysis (TGA) and Differential Scanning Calorimetry Analysis (DSC) thermograms are shown in FIG. 7B.

In one embodiment, crystalline Form VII of Compound (I) is an anhydrate.

TABLE 7
Representative XRPD peaks of crystalline
Form VII of Compound (I)

| Peak | Angle | Rel. Intensity |
|---|---|---|
| 1 | 5.286° | 63.7% |
| 2 | 5.645° | 18.4% |
| 3 | 7.591° | 5.5% |
| 4 | 8.155° | 14.6% |
| 5 | 10.245° | 100.0% |
| 6 | 14.535° | 6.1% |
| 7 | 14.870° | 6.7% |
| 8 | 15.238° | 7.9% |
| 9 | 17.003° | 4.8% |
| 10 | 17.278° | 6.3% |
| 11 | 19.267° | 20.5% |
| 12 | 20.576° | 8.5% |
| 13 | 22.384° | 2.4% |
| 14 | 23.856° | 11.4% |
| 15 | 24.570° | 4.1% |
| 16 | 27.922° | 6.6% |

### Salt Forms of Compound (I)

### Maleate Salt

In one embodiment, the present disclosure provides crystalline Form I of maleate salt of Compound (I), wherein the molar ratio between Compound (I) and maleic acid is 1:1, which is characterized by XRPD peaks at 5.7°, 9.3°, 10.3°, 13.9°, and 20.7°±0.2 in 2θ. In another embodiment, the present disclosure provides a crystalline Form I of maleate salt of Compound (I), wherein the molar ratio between Compound (I) and maleic acid is 1:1, which is characterized by XRPD peaks at 5.7°, 9.3°, 10.3°, 13.9°, 20.7°, 22.4°, 24.1°, 24.5°, and 26.8°±0.2 in 2θ.

In another embodiment, the present disclosure provides a crystalline Form I of maleate salt of Compound (I), wherein the molar ratio between Compound (I) and maleic acid is 1:1, which is characterized by differential scanning calorimeter (DSC) peak phase transition temperature of 173.9±2° C.

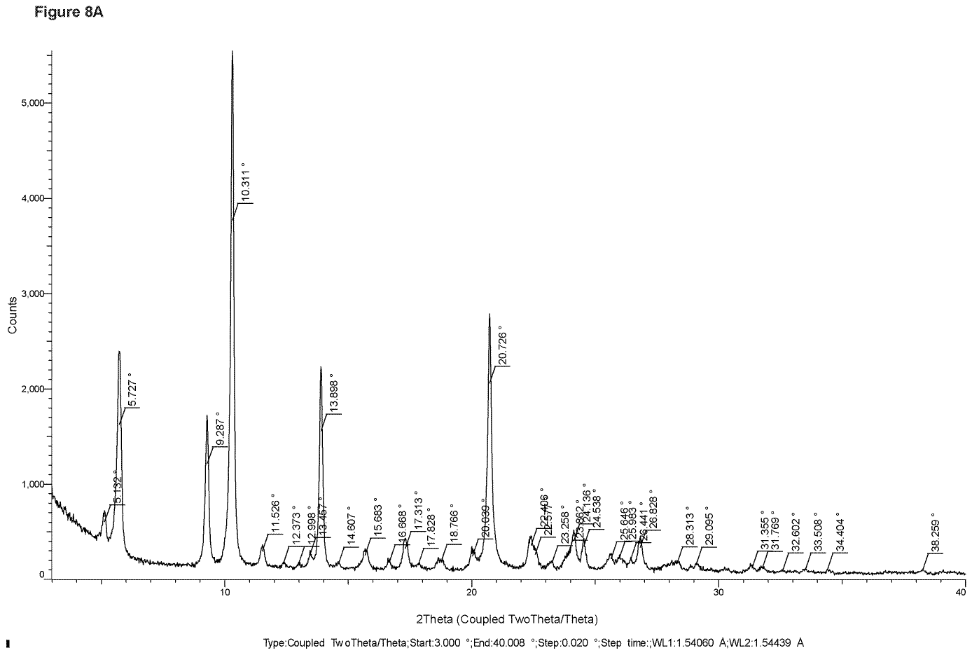

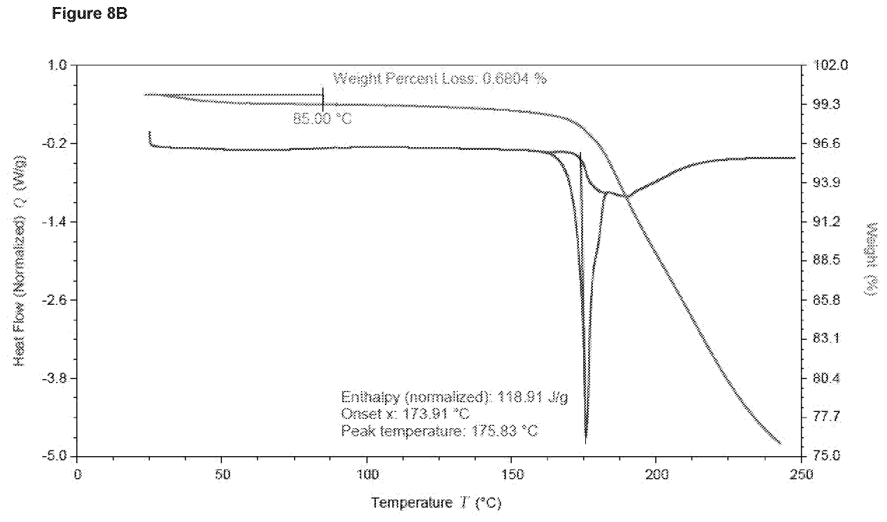

Representative XRPD peaks are tabulated in Table 8. The XRPD patterns and peaks are shown in FIG. 8A. The Thermogravimetric Analysis (TGA) and Differential Scanning Calorimetry Analysis (DSC) thermograms are shown in FIG. 8B.

In one embodiment, crystalline Form I of maleate salt of Compound (I) is an anhydrate.

TABLE 8
Representative XRPD peaks of crystalline
Form I of Maleate Salt of Compound (I)

| Peak | Angle | Rel. Intensity |
|---|---|---|
| 1 | 5.132° | 5.7% |
| 2 | 5.727° | 36.4% |
| 3 | 9.287° | 29.3% |
| 4 | 10.311° | 100.0% |
| 5 | 11.526° | 4.2% |
| 6 | 12.373° | 1.1% |
| 7 | 12.998° | 1.3% |
| 8 | 13.457° | 3.8% |
| 9 | 13.898° | 39.6% |
| 10 | 14.607° | 1.1% |
| 11 | 15.683° | 3.8% |
| 12 | 16.668° | 2.0% |
| 13 | 17.313° | 5.8% |
| 14 | 17.828° | 0.9% |
| 15 | 18.766° | 2.3% |
| 16 | 20.039° | 3.6% |
| 17 | 20.726° | 53.6% |
| 18 | 22.406° | 6.3% |
| 19 | 22.577° | 3.8% |
| 20 | 23.258° | 1.5% |
| 21 | 23.862° | 3.2% |
| 22 | 24.136° | 7.9% |
| 23 | 24.538° | 6.3% |
| 24 | 25.646° | 2.8% |
| 25 | 25.983° | 2.8% |
| 26 | 26.441° | 2.5% |
| 27 | 26.828° | 6.1% |
| 28 | 28.313° | 1.5% |
| 29 | 29.095° | 1.7% |
| 30 | 31.355° | 1.1% |
| 31 | 31.769° | 1.1% |
| 32 | 32.602° | 0.7% |
| 33 | 33.508° | 0.8% |
| 34 | 34.404° | 1.0% |
| 35 | 38.259° | 0.9% |

### Tosylate Salt

In one embodiment, the present disclosure provides crystalline Form I of tosylate salt of Compound (I), wherein the molar ratio between Compound (I) and p-toluenesulfonic acid is 1:1, which is characterized by XRPD peaks at 9.5°, 11.0°, 14.1°, 21.4°, and 21.9°±0.2 in 2θ. In another embodiment, the present disclosure provides a crystalline Form I of tosylate salt of Compound (I), wherein the molar ratio between Compound (I) and p-toluenesulfonic acid is 1:1, which is characterized by XRPD peaks at 7.2°, 9.5°, 11.0°, 14.10, 21.1°, 21.4°, 21.9°, 22.6°, and 24.6°±0.2 in 2θ.

In another embodiment, the present disclosure provides a crystalline Form I of tosylate salt of Compound (I), wherein the molar ratio between Compound (I) and p-toluenesulfonic acid is 1:1, which is characterized by differential scanning calorimeter (DSC) peak phase transition temperatures of 38.9±2° C. and 203.0±2° C.

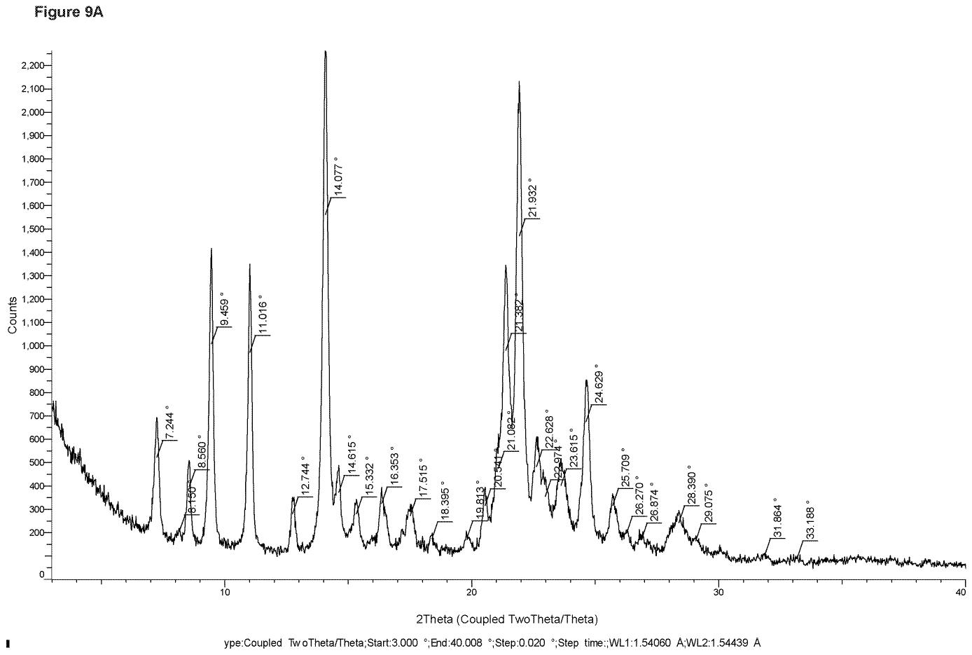

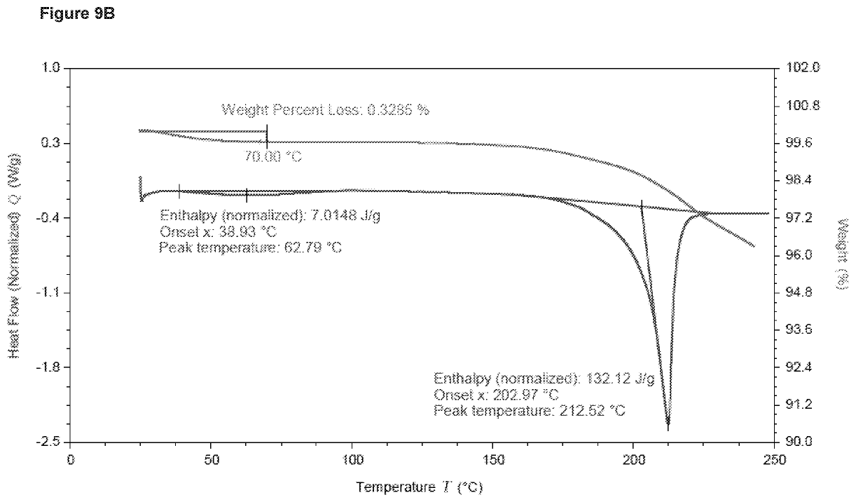

Representative XRPD peaks are tabulated in Table 9. The XRPD patterns and peaks are shown in FIG. 9A. The Thermogravimetric Analysis (TGA) and Differential Scanning Calorimetry Analysis (DSC) thermograms are shown in FIG. 9B.

In one embodiment, crystalline Form I of tosylate salt of Compound (I) is an anhydrate.

TABLE 9
Representative XRPD peaks of crystalline
Form I of Tosylate Salt of Compound (I)

| Peak | Angle | Rel. Intensity |
|---|---|---|
| 1 | 7.244° | 23.3% |
| 2 | 8.150° | 2.3% |
| 3 | 8.560° | 16.1% |
| 4 | 9.459° | 59.6% |
| 5 | 11.016° | 58.7% |
| 6 | 12.744° | 11.1% |
| 7 | 14.077° | 100.0% |
| 8 | 14.615° | 16.3% |
| 9 | 15.332° | 9.7% |
| 10 | 16.353° | 12.6% |
| 11 | 17.515° | 9.3% |
| 12 | 18.395° | 3.2% |
| 13 | 19.813° | 3.4% |
| 14 | 20.541° | 11.8% |
| 15 | 21.082° | 22.1% |
| 16 | 21.382° | 57.1% |
| 17 | 21.932° | 90.8% |
| 18 | 22.628° | 20.9% |
| 19 | 22.974° | 11.8% |
| 20 | 23.615° | 15.0% |
| 21 | 24.629° | 34.6% |
| 22 | 25.709° | 10.7% |
| 23 | 26.270° | 3.2% |
| 24 | 26.874° | 2.3% |
| 25 | 28.390° | 8.7% |
| 26 | 29.075° | 3.7% |
| 27 | 31.864° | 1.9% |
| 28 | 33.188° | 1.6% |

### Hydrochloride Salt Form I

In one embodiment, the present disclosure provides crystalline Form I of hydrochloride salt of Compound (I), wherein the molar ratio between Compound (I) and hydrochloric acid is 1:1, which is characterized by XRPD peaks at 5.0°, 9.7°, 10.2°, 16.3°, 20.7°, 21.3°, 23.7°, and 24.5°±0.2 in 2θ.

In another embodiment, the present disclosure provides a crystalline Form I of hydrochloride of Compound (I), wherein the molar ratio between Compound (I) and hydrochloric acid is 1:1, which is characterized by differential scanning calorimeter (DSC) peak phase transition temperature of 244.1±2° C.

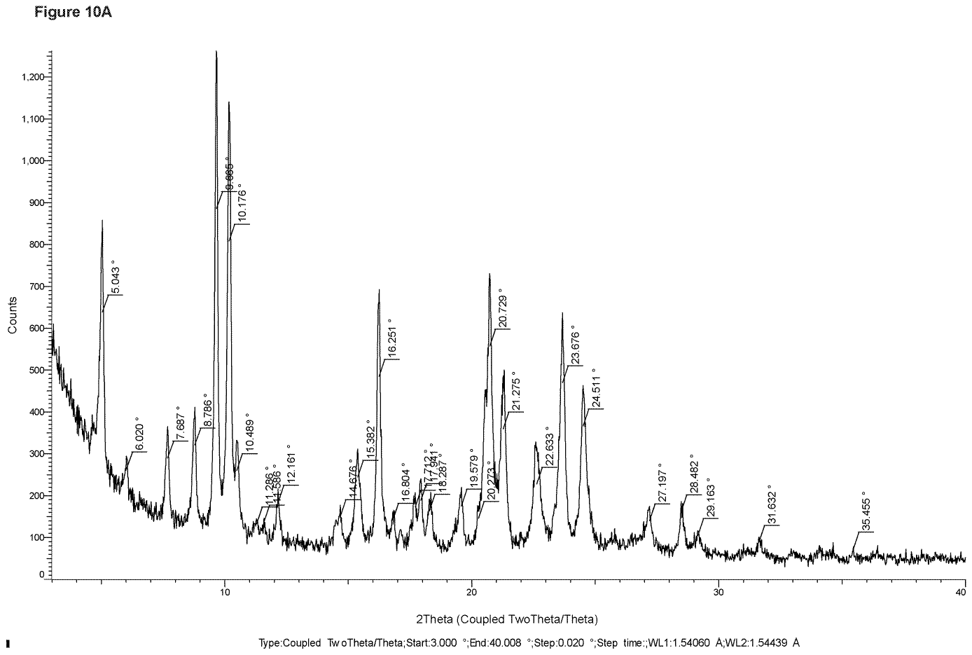

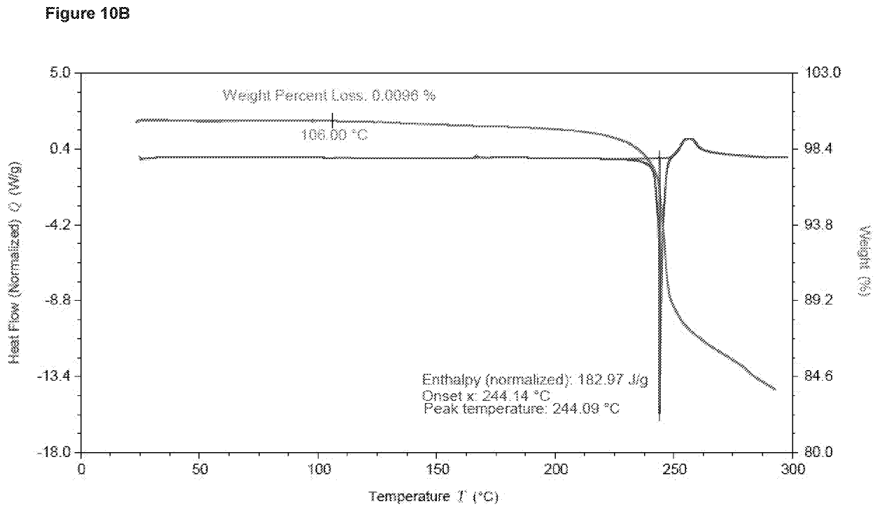

Representative XRPD peaks are tabulated in Table 10. The XRPD patterns and peaks are shown in FIG. 10A. The Thermogravimetric Analysis (TGA) and Differential Scanning Calorimetry Analysis (DSC) thermograms are shown in FIG. 10B.

TABLE 10
Representative XRPD peaks of crystalline Form
I of Hydrochloride Salt of Compound (I)

| Peak | Angle | Rel. Intensity |
|---|---|---|
| 1 | 5.043° | 46.9% |
| 2 | 6.020° | 7.9% |
| 3 | 7.687° | 19.8% |
| 4 | 8.786° | 25.0% |
| 5 | 9.665° | 100.0% |
| 6 | 10.176° | 90.6% |
| 7 | 10.489° | 19.3% |
| 8 | 11.286° | 4.3% |
| 9 | 11.586° | 5.5% |
| 10 | 12.161° | 12.6% |
| 11 | 14.676° | 8.7% |
| 12 | 15.382° | 20.3% |
| 13 | 16.251° | 51.4% |
| 14 | 16.804° | 6.4% |
| 15 | 17.712° | 11.1% |
| 16 | 17.941° | 13.4% |
| 17 | 18.287° | 10.5% |
| 18 | 19.579° | 12.2% |
| 19 | 20.273° | 7.6% |
| 20 | 20.729° | 61.3% |
| 21 | 21.275° | 35.3% |
| 22 | 22.633° | 17.6% |
| 23 | 23.676° | 49.4% |
| 24 | 24.511° | 36.7% |
| 25 | 27.197° | 8.3% |
| 26 | 28.482° | 12.4% |
| 27 | 29.163° | 5.6% |
| 28 | 31.632° | 4.8% |
| 29 | 35.455° | 3.7% |

### Hydrochloride Salt Form II

In one embodiment, the present disclosure provides crystalline Form II of hydrochloride salt of Compound (I), wherein the molar ratio between Compound (I) and hydrochloric acid is 1:1, which is characterized by XRPD peaks at 4.7°, 7.6°, 9.5°, 10.7°, 14.3°, 15.3°, 17.1°, 19.9° and 24.7°±0.2 in 2θ.

In another embodiment, the present disclosure provides a crystalline Form II of hydrochloride salt of Compound (I), wherein the molar ratio between Compound (I) and hydrochloric acid is 1:1, which is characterized by differential scanning calorimeter (DSC) peak phase transition temperatures of 62.7±2° C., 219.6±2° C., and 221.7±2° C.

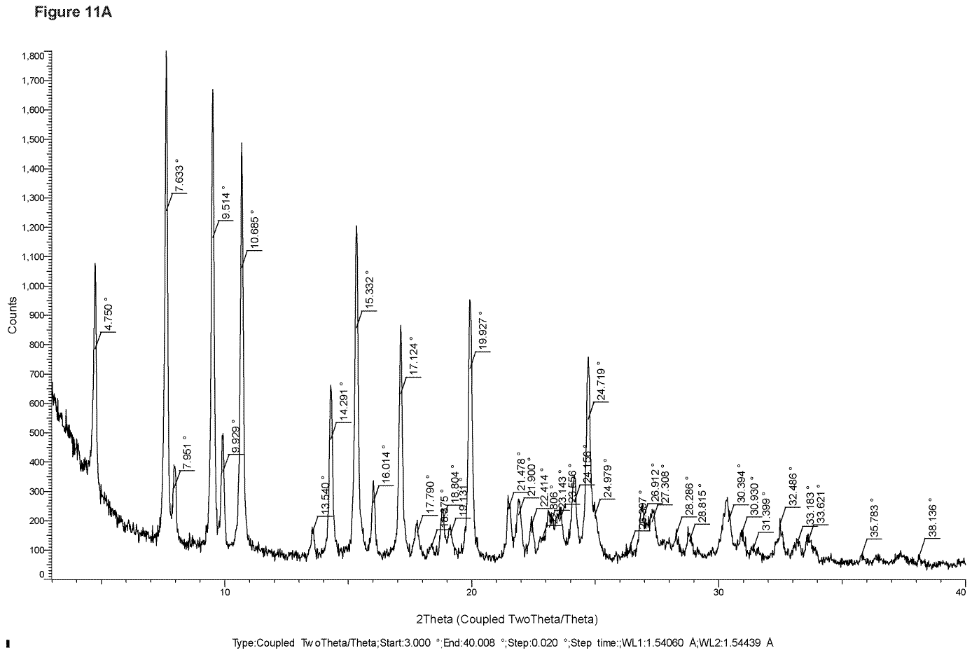

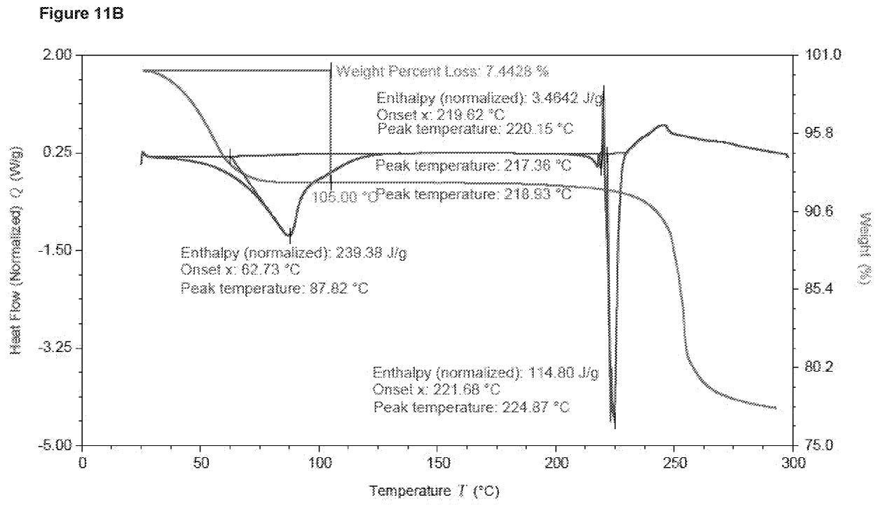

Representative XRPD peaks are tabulated in Table 11. The XRPD patterns and peaks are shown in FIG. 11A. The Thermogravimetric Analysis (TGA) and Differential Scanning Calorimetry Analysis (DSC) thermograms are shown in FIG. 11B.

TABLE 11
Representative XRPD peaks of crystalline Form
II of Hydrochloride Salt of Compound (I)

| Peak | Angle | Rel. Intensity |
|---|---|---|
| 1 | 4.750° | 41.9% |
| 2 | 7.633° | 100.0% |
| 3 | 7.951° | 16.1% |
| 4 | 9.514° | 93.3% |
| 5 | 9.929° | 22.5% |
| 6 | 10.685° | 85.1% |
| 7 | 13.540° | 6.5% |
| 8 | 14.291° | 34.6% |
| 9 | 15.332° | 68.4% |
| 10 | 16.014° | 15.8% |
| 11 | 17.124° | 49.0% |
| 12 | 17.790° | 7.2% |
| 13 | 18.375° | 2.9% |
| 14 | 18.804° | 10.2% |
| 15 | 19.131° | 6.2% |
| 16 | 19.927° | 57.0% |
| 17 | 21.478° | 14.1% |
| 18 | 21.900° | 11.9% |
| 19 | 22.414° | 8.3% |
| 20 | 22.806° | 2.8% |
| 21 | 23.143° | 7.4% |
| 22 | 23.556° | 9.2% |
| 23 | 24.156° | 15.4% |
| 24 | 24.719° | 41.0% |
| 25 | 24.979° | 11.9% |
| 26 | 26.397° | 2.4% |
| 27 | 26.912° | 10.2% |
| 28 | 27.308° | 9.6% |
| 29 | 28.286° | 5.8% |
| 30 | 28.815° | 3.9% |
| 31 | 30.394° | 10.0% |
| 32 | 30.930° | 6.4% |
| 33 | 31.399° | 1.9% |
| 34 | 32.486° | 10.0% |
| 35 | 33.183° | 4.5% |
| 36 | 33.621° | 5.3% |

### Hydrochloride Salt Form III

In one embodiment, the present disclosure provides crystalline Form III of hydrochloride salt of Compound (I), wherein the molar ratio between Compound (I) and hydrochloric acid is 1:2, which is characterized by XRPD peaks at 3.6°, 10.2°, 11.2°, 12.5°, and 16.3°±0.2 in 2θ.

In another embodiment, the present disclosure provides a crystalline Form III of hydrochloride salt of Compound (I), wherein the molar ratio between Compound (I) and hydrochloric acid is 1:2, which is characterized by differential scanning calorimeter (DSC) peak phase transition temperature of 28.8±2° C., 166.9±2° C., and 217.25±2° C.

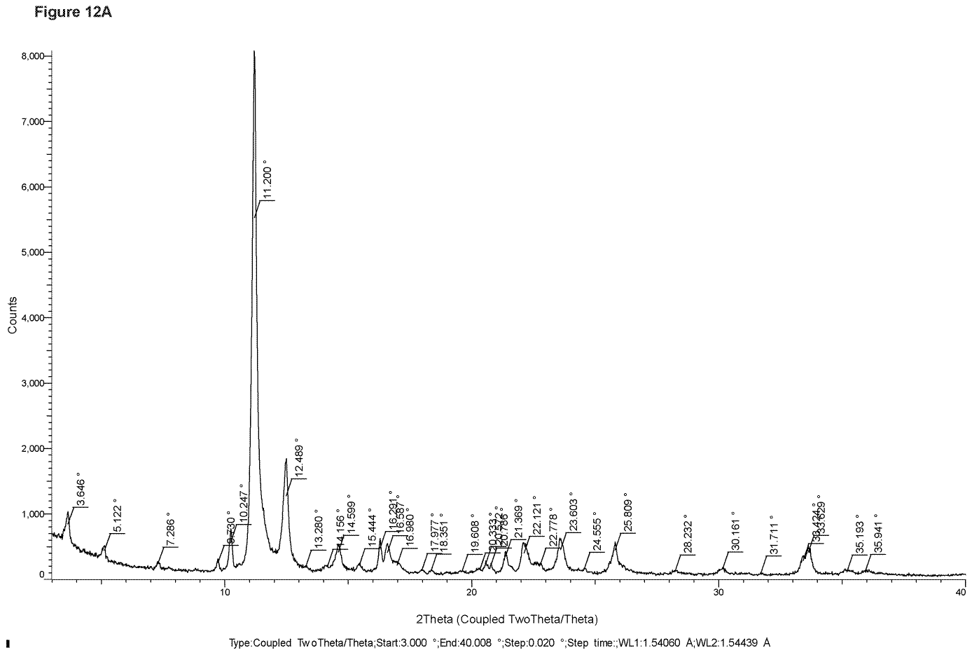

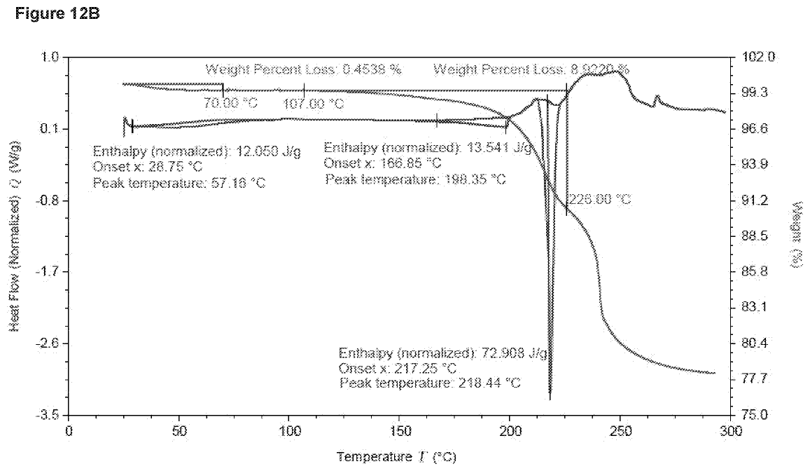

Representative XRPD peaks are tabulated in Table 12. The XRPD patterns and peaks are shown in FIG. 12A. The Thermogravimetric Analysis (TGA) and Differential Scanning Calorimetry Analysis (DSC) thermograms are shown in FIG. 12B.

TABLE 12
Representative XRPD peaks of crystalline Form
III of Hydrochloride Salt of Compound (I)

| Peak | Angle | Rel. Intensity |
|---|---|---|
| 1 | 3.646° | 5.3% |
| 2 | 5.122° | 2.3% |
| 3 | 7.286° | 1.4% |
| 4 | 9.730° | 2.3% |
| 5 | 10.247° | 8.0% |
| 6 | 11.200° | 100.0% |
| 7 | 12.489° | 20.8% |
| 8 | 13.280° | 0.8% |
| 9 | 14.156° | 1.1% |
| 10 | 14.599° | 5.2% |
| 11 | 15.444° | 1.5% |
| 12 | 16.291° | 6.6% |
| 13 | 16.587° | 5.5% |
| 14 | 16.980° | 2.0% |
| 15 | 17.977° | 0.8% |
| 16 | 18.351° | 0.8% |
| 17 | 19.608° | 0.4% |
| 18 | 20.333° | 0.8% |
| 19 | 20.572° | 2.6% |
| 20 | 20.786° | 2.1% |
| 21 | 21.369° | 4.4% |
| 22 | 22.121° | 5.1% |
| 23 | 22.778° | 1.6% |
| 24 | 23.603° | 6.1% |
| 25 | 24.555° | 0.9% |
| 26 | 25.809° | 6.5% |
| 27 | 28.232° | 0.8% |
| 28 | 30.161° | 1.5% |
| 29 | 31.711° | 0.6% |
| 30 | 33.424° | 3.8% |
| 31 | 33.629° | 6.2% |
| 32 | 35.193° | 0.6% |
| 33 | 35.941° | 0.7% |

### Hydrochloride Salt Form IV

In one embodiment, the present disclosure provides crystalline Form IV of hydrochloride salt of Compound (I), wherein the molar ratio between Compound (I) and hydrochloric acid is 1:2, which is characterized by XRPD peaks at 6.1°, 9.4°, 12.2°, 17.2°, 19.9°, and 22.7°±0.2 in 2θ.

In another embodiment, the present disclosure provides a crystalline Form IV of hydrochloride salt of Compound (I), wherein the molar ratio between Compound (I) and hydrochloric acid is 1:2, which is characterized by differential scanning calorimeter (DSC) peak phase transition temperature of 45.7±2° C. and 172.6±2° C.

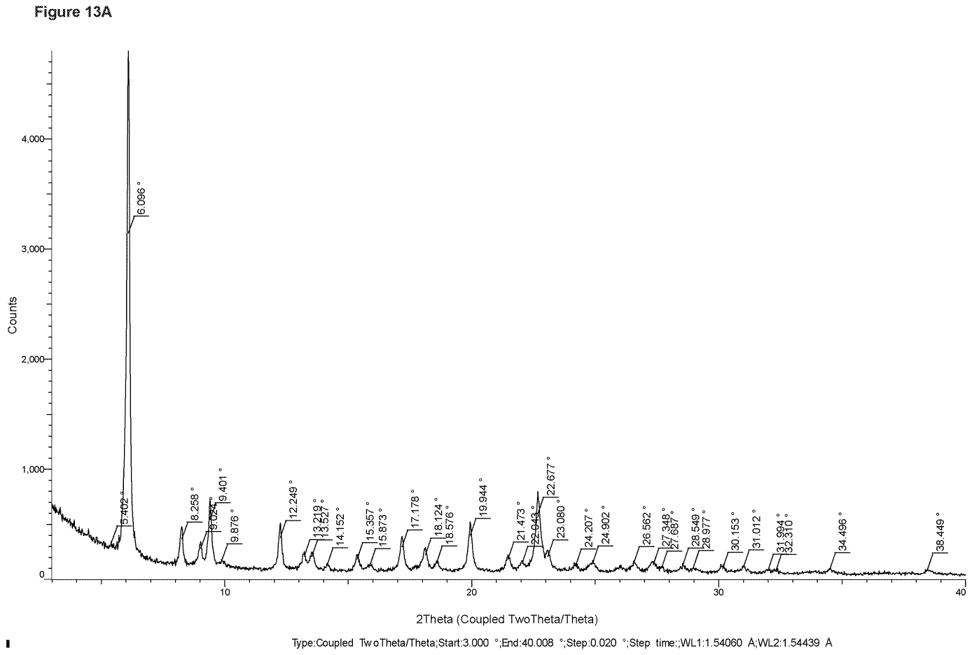

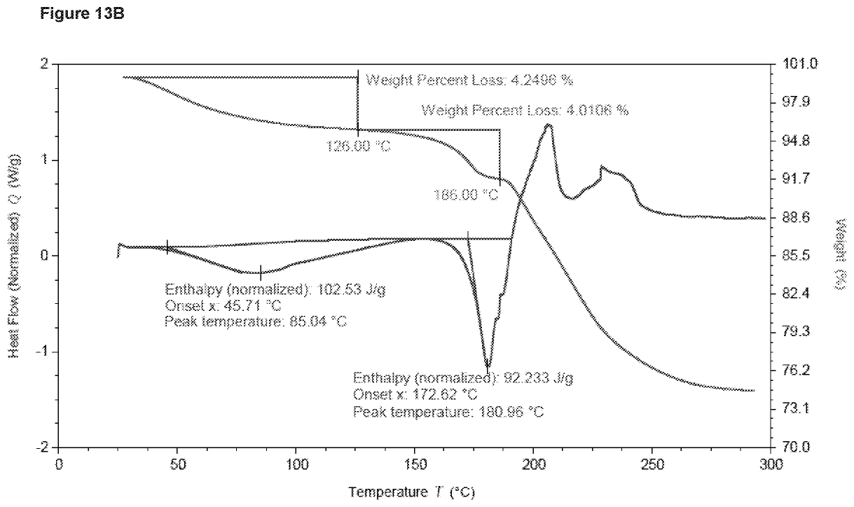

Representative XRPD peaks are tabulated in Table 13. The XRPD patterns and peaks are shown in FIG. 13A. The Thermogravimetric Analysis (TGA) and Differential Scanning Calorimetry Analysis (DSC) thermograms are shown in FIG. 13B.

TABLE 13
Representative XRPD peaks of crystalline Form
IV of Hydrochloride Salt of Compound (I)

| Peak | Angle | Rel. Intensity |
|---|---|---|
| 1 | 5.402° | 1.4% |
| 2 | 6.096° | 100.0% |
| 3 | 8.258° | 7.6% |
| 4 | 9.024° | 5.0% |
| 5 | 9.401° | 14.1% |
| 6 | 9.876° | 1.6% |
| 7 | 12.249° | 9.8% |
| 8 | 13.219° | 3.8% |
| 9 | 13.527° | 3.8% |
| 10 | 14.152° | 1.6% |
| 11 | 15.357° | 3.6% |
| 12 | 15.873° | 1.7% |
| 13 | 17.178° | 7.1% |
| 14 | 18.124° | 4.5% |
| 15 | 18.576° | 2.1% |
| 16 | 19.944° | 10.9% |
| 17 | 21.473° | 3.4% |
| 18 | 22.043° | 1.9% |
| 19 | 22.677° | 17.2% |
| 20 | 23.080° | 3.9% |
| 21 | 24.207° | 1.2% |
| 22 | 24.902° | 2.4% |
| 23 | 26.562° | 2.3% |
| 24 | 27.348° | 1.8% |
| 25 | 27.687° | 0.9% |
| 26 | 28.549° | 1.9% |
| 27 | 28.977° | 1.3% |
| 28 | 30.153° | 0.9% |
| 29 | 31.012° | 1.7% |
| 30 | 31.994° | 0.7% |
| 31 | 32.310° | 0.6% |
| 32 | 34.496° | 1.2% |
| 33 | 38.449° | 1.2% |

### Hydrobromide Salt Form I

In one embodiment, the present disclosure provides crystalline Form I of hydrobromide salt of Compound (I), wherein the molar ratio between Compound (I) and HBr is 1:1, which is characterized by XRPD peaks at 4.9°, 9.0°, 11.4°, 11.8°, 14.7, 15.9, 18.1, and 22.6°±0.2 in 2θ.

In another embodiment, the present disclosure provides a crystalline Form I of hydrobromide salt of Compound (I), wherein the molar ratio between Compound (I) and hydrobromic acid is 1:1, which is characterized by differential scanning calorimeter (DSC) peak phase transition temperature of 199.8±2° C.

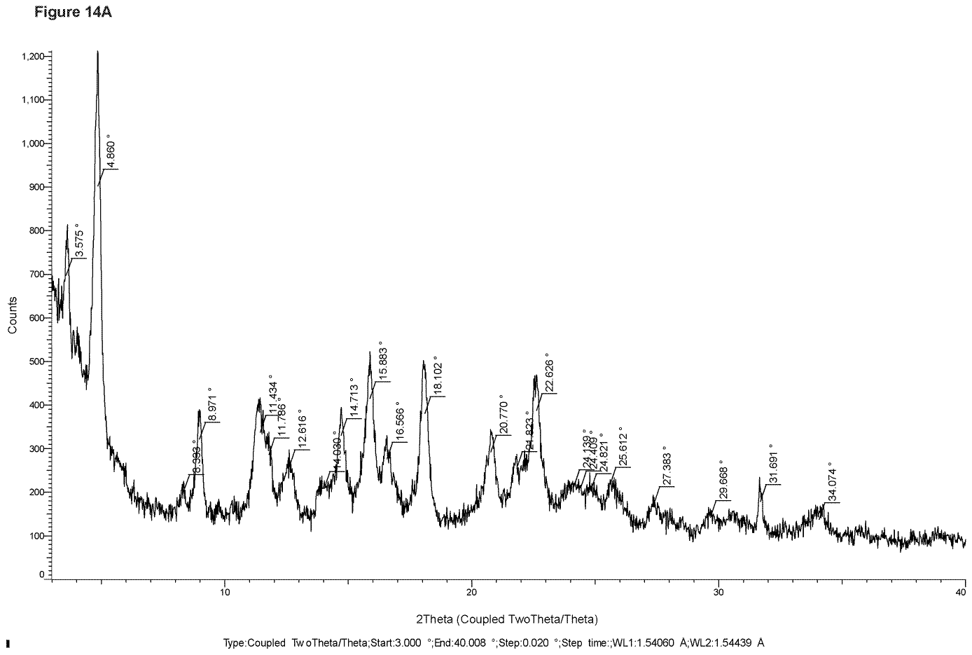

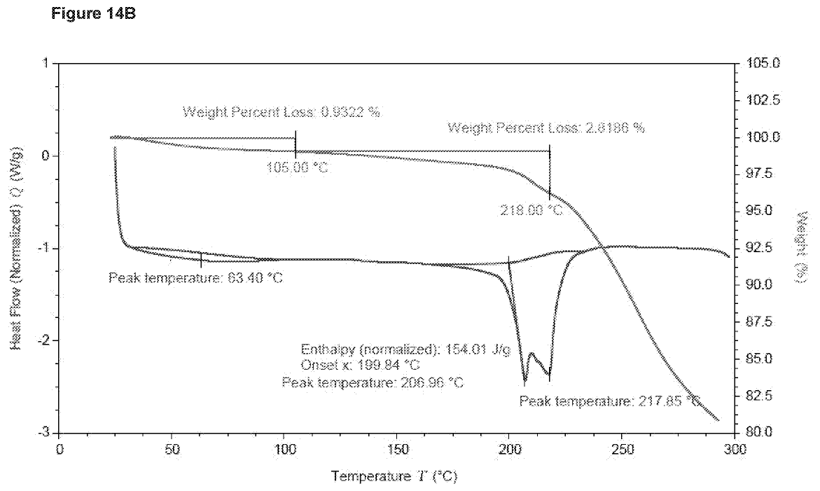

Representative XRPD peaks are tabulated in Table 14. The XRPD patterns and peaks are shown in FIG. 14A. The Thermogravimetric Analysis (TGA) and Differential Scanning Calorimetry Analysis (DSC) thermograms are shown in FIG. 14B.

TABLE 14
Representative XRPD peaks of crystalline
Form I of HBr Salt of Compound (I)

| Peak | Angle | Rel. Intensity |
|---|---|---|
| 1 | 3.575° | 22.9% |
| 2 | 4.860° | 100.0% |
| 3 | 8.333° | 8.3% |
| 4 | 8.971° | 32.1% |
| 5 | 11.434° | 34.1% |
| 6 | 11.786° | 24.0% |
| 7 | 12.616° | 19.2% |
| 8 | 14.030° | 9.1% |
| 9 | 14.713° | 31.5% |
| 10 | 15.883° | 47.2% |
| 11 | 16.566° | 20.7% |
| 12 | 18.102° | 44.3% |
| 13 | 20.770° | 23.6% |
| 14 | 21.823° | 14.4% |
| 15 | 22.626° | 39.7% |
| 16 | 24.139° | 6.8% |
| 17 | 24.409° | 6.7% |
| 18 | 24.821° | 6.3% |
| 19 | 25.612° | 11.5% |
| 20 | 27.383° | 9.0% |
| 21 | 29.668° | 6.4% |
| 22 | 31.691° | 12.6% |
| 23 | 34.074° | 5.7% |

### Sulfate Salt Form I

In one embodiment, the present disclosure provides crystalline Form I of sulfate salt of Compound (I), wherein the molar ratio between Compound (I) and sulfuric acid is 1:1, which is characterized by XRPD peaks at 4.9°, 7.9°, 10.5°, 12.3°, 14.7°, 16.6° and 19.4°±0.2 in 2θ.

In another embodiment, the present disclosure provides a crystalline Form I of sulfate salt of Compound (I), wherein the molar ratio between Compound (I) and sulfuric acid is 1:1, which is characterized by differential scanning calorimeter (DSC) peak phase transition temperature of 103.2±2° C. and 196.4±2° C.

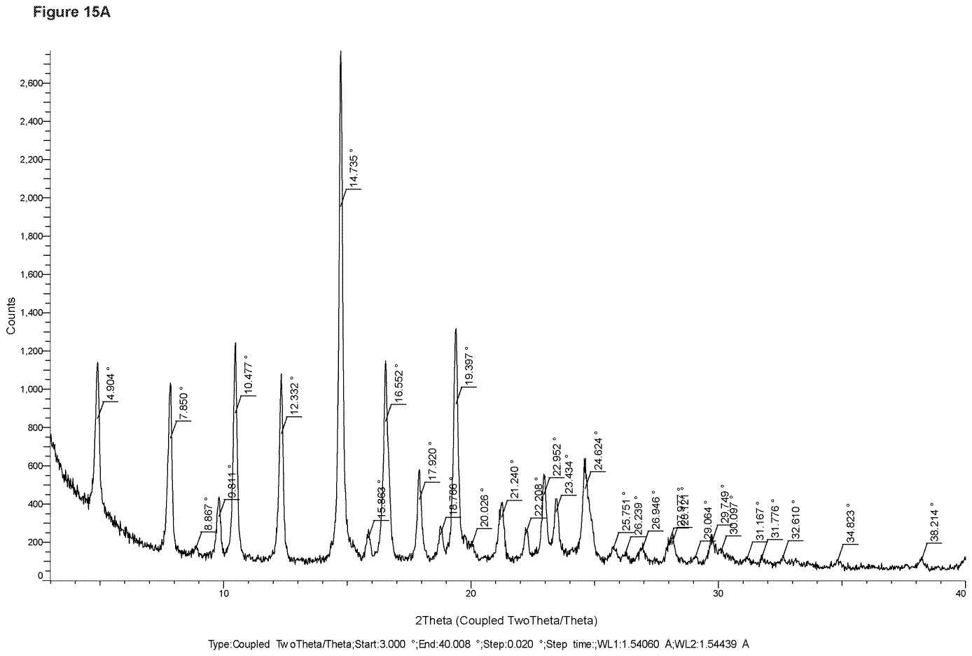

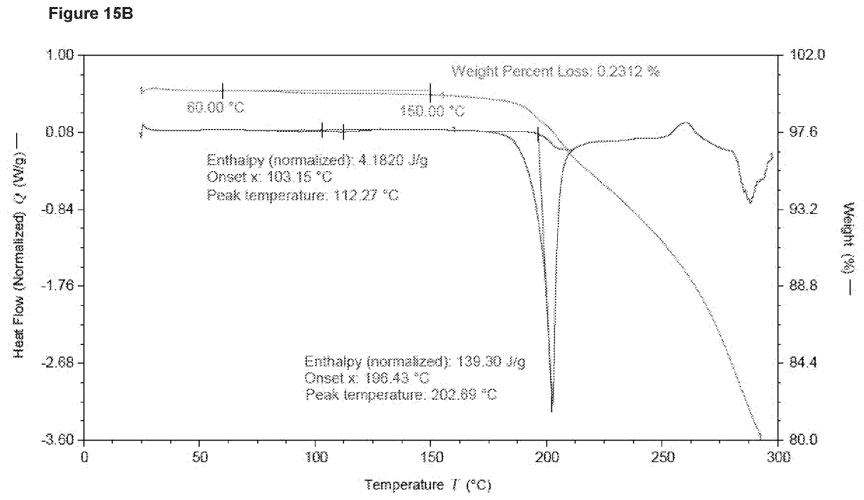

Representative XRPD peaks are tabulated in Table 15. The XRPD patterns and peaks are shown in FIG. 15A. The Thermogravimetric Analysis (TGA) and Differential Scanning Calorimetry Analysis (DSC) thermograms are shown in FIG. 15B.

TABLE 15
Representative XRPD peaks of crystalline
Form I of Sulfate Salt of Compound (I)

| Peak | Angle | Rel. Intensity |
|---|---|---|
| 1 | 4.904° | 25.8% |
| 2 | 7.850° | 31.9% |
| 3 | 8.867° | 1.6% |
| 4 | 9.811° | 11.2% |
| 5 | 10.477° | 40.9% |
| 6 | 12.332° | 35.7% |
| 7 | 14.735° | 100.0% |
| 8 | 15.863° | 5.6% |
| 9 | 16.552° | 38.6% |
| 10 | 17.920° | 16.9% |
| 11 | 18.766° | 6.9% |
| 12 | 19.397° | 43.8% |
| 13 | 20.026° | 3.7% |
| 14 | 21.240° | 11.9% |
| 15 | 22.208° | 6.7% |
| 16 | 22.952° | 17.7% |
| 17 | 23.434° | 12.6% |
| 18 | 24.624° | 19.6% |
| 19 | 25.751° | 2.9% |
| 20 | 26.239° | 1.9% |
| 21 | 26.946° | 3.7% |
| 22 | 27.977° | 4.8% |
| 23 | 28.121° | 5.1% |
| 24 | 29.064° | 1.4% |
| 25 | 29.749° | 5.8% |
| 26 | 30.097° | 2.7% |
| 27 | 31.167° | 1.2% |
| 28 | 31.776° | 2.1% |
| 29 | 32.610° | 2.0% |
| 30 | 34.826° | 1.8% |
| 31 | 38.214° | 2.5% |

### Phosphate Salt Form I

In one embodiment, the present disclosure provides crystalline Form I of phosphate salt of Compound (I), wherein the molar ratio between Compound (I) and phosphoric acid is 1:1, which is characterized by XRPD peaks at 6.7°, 13.7°, 19.2°, 22.5°, and 25.1°±0.2 in 2θ.

In another embodiment, the present disclosure provides a crystalline Form I of phosphate salt of Compound (I), wherein the molar ratio between Compound (I) and phosphoric acid is 1:1, which is characterized by differential scanning calorimeter (DSC) peak phase transition temperature of 203.3±2° C.

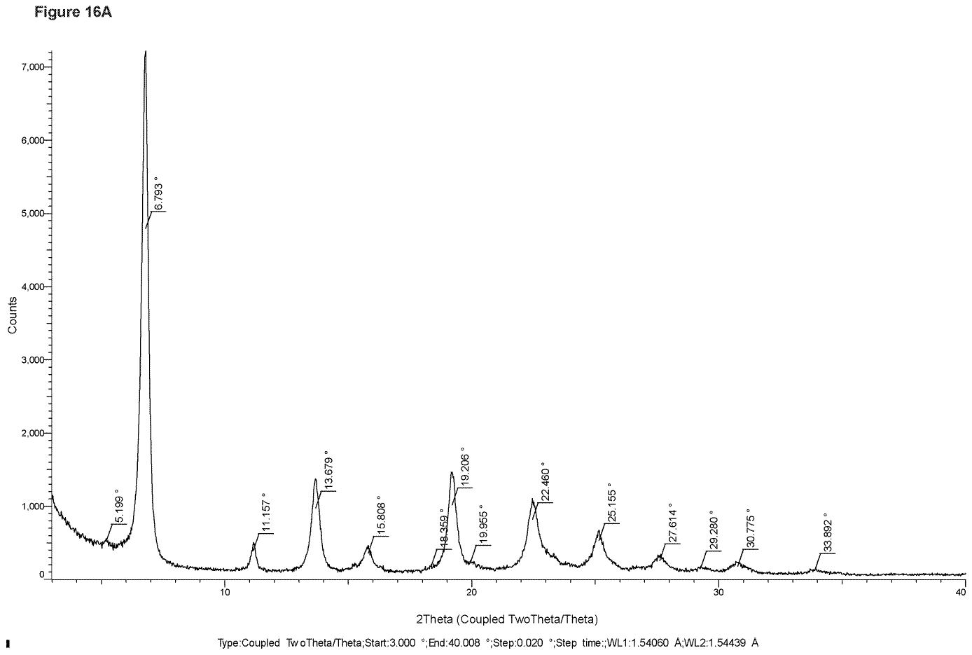

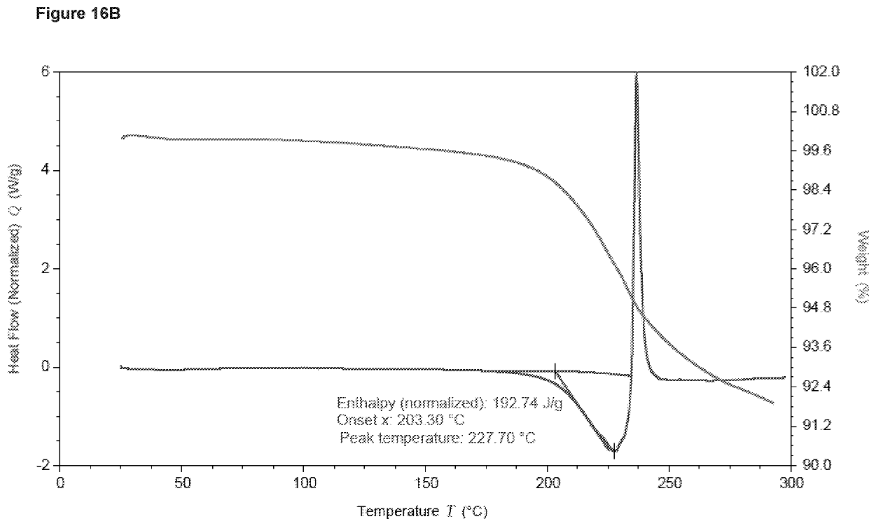

Representative XRPD peaks are tabulated in Table 16. The XRPD patterns and peaks are shown in FIG. 16A. The Thermogravimetric Analysis (TGA) and Differential Scanning Calorimetry Analysis (DSC) thermograms are shown in FIG. 16B.

TABLE 16
Representative XRPD peaks of crystalline
Form I of Phosphate Salt of Compound (I)

| Peak | Angle | Rel. Intensity |
|---|---|---|
| 1 | 5.199° | 1.2% |
| 2 | 6.793° | 100.0% |
| 3 | 11.157° | 6.1% |
| 4 | 13.679° | 18.8% |
| 5 | 15.808° | 5.1% |
| 6 | 18.359° | 1.2% |
| 7 | 19.206° | 19.7% |
| 8 | 19.955° | 2.1% |
| 9 | 22.460° | 14.5% |
| 10 | 25.155° | 8.1% |
| 11 | 27.614° | 2.6% |
| 12 | 29.280° | 0.9% |
| 13 | 30.775° | 1.6% |
| 14 | 33.892° | 0.9% |

### Tartrate Salt Form I

In one embodiment, the present disclosure provides crystalline Form I of tartrate salt of Compound (I), wherein the molar ratio between Compound (I) and tartaric acid is 1:1, which is characterized by XRPD peaks at 4.8°, 9.5°, 10.0°, 14.4°, 16.1°, and 20.0°±0.2 in 2θ.

In another embodiment, the present disclosure provides a crystalline Form I of tartrate salt of Compound (I), wherein the molar ratio between Compound (I) and tartaric acid is 1:1, which is characterized by differential scanning calorimeter (DSC) peak phase transition temperature of 36.3±2° C. and 165.5±2° C.

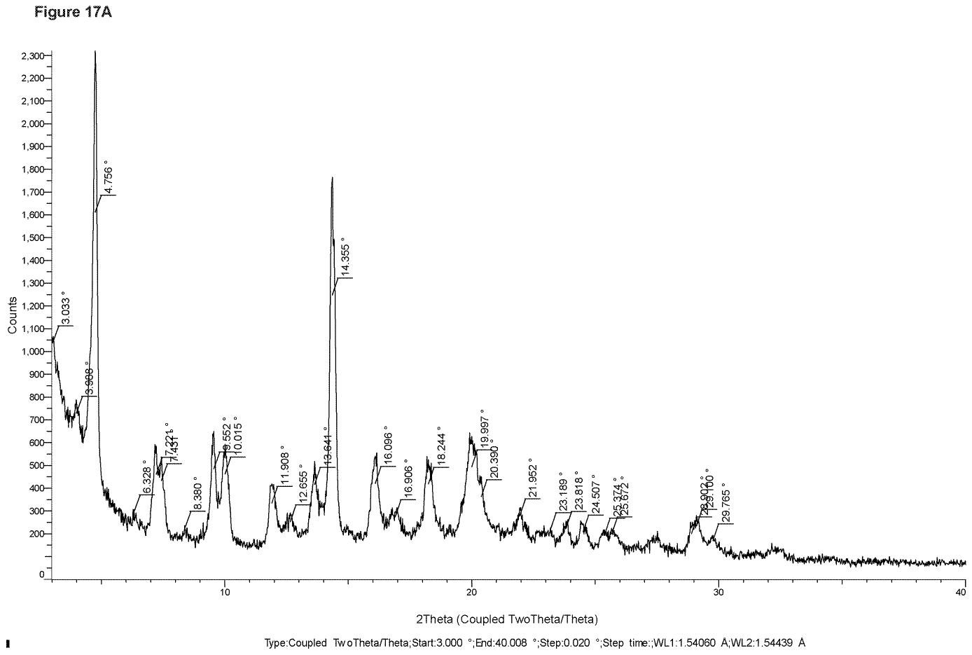

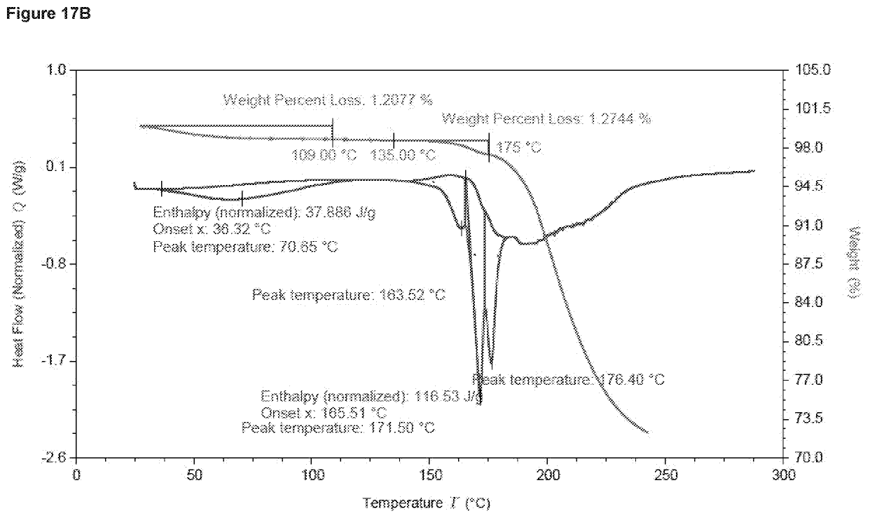

Representative XRPD peaks are tabulated in Table 17. The XRPD patterns and peaks are shown in FIG. 17A. The Thermogravimetric Analysis (TGA) and Differential Scanning Calorimetry Analysis (DSC) thermograms are shown in FIG. 17B.

TABLE 17
Representative XRPD peaks of crystalline
Form I of Tartrate Salt of Compound (I)

| Peak | Angle | Rel. Intensity |
|---|---|---|
| 1 | 3.033° | −1.9% |
| 2 | 3.988° | 8.3% |
| 3 | 4.756° | 100.0% |
| 4 | 6.328° | 5.3% |
| 5 | 7.221° | 22.5% |
| 6 | 7.431° | 20.6% |
| 7 | 8.380° | 4.1% |
| 8 | 9.552° | 27.6% |
| 9 | 10.015° | 26.0% |
| 10 | 11.908° | 15.3% |
| 11 | 12.655° | 6.8% |
| 12 | 13.641° | 20.1% |
| 13 | 14.356° | 92.3% |
| 14 | 16.096° | 20.2% |
| 15 | 16.906° | 7.4% |
| 16 | 18.244° | 19.6% |
| 17 | 19.997° | 25.3% |
| 18 | 20.390° | 14.0% |
| 19 | 21.952° | 8.1% |
| 20 | 23.189° | 4.7% |
| 21 | 23.818° | 5.7% |
| 22 | 24.507° | 5.0% |
| 23 | 25.374° | 4.4% |
| 24 | 25.672° | 5.0% |
| 25 | 28.902° | 6.7% |
| 26 | 29.100° | 9.7% |
| 27 | 29.765° | 4.7% |

### Fumarate Acid Form I

In one embodiment, the present disclosure provides crystalline Form I of fumarate salt of Compound (I), wherein the molar ratio between Compound (I) and fumaric acid is 1:1, which is characterized by XRPD peaks at 6.0°, 9.9°, 12.9°, 15.3°, 15.9°, 19.7°, and 25.1°±0.2 in 2θ.

In another embodiment, the present disclosure provides a crystalline Form I of fumarate salt of Compound (I), wherein the molar ratio between Compound (I) and fumaric acid is 1:1, which is characterized by differential scanning calorimeter (DSC) peak phase transition temperature of 181.5±2° C.

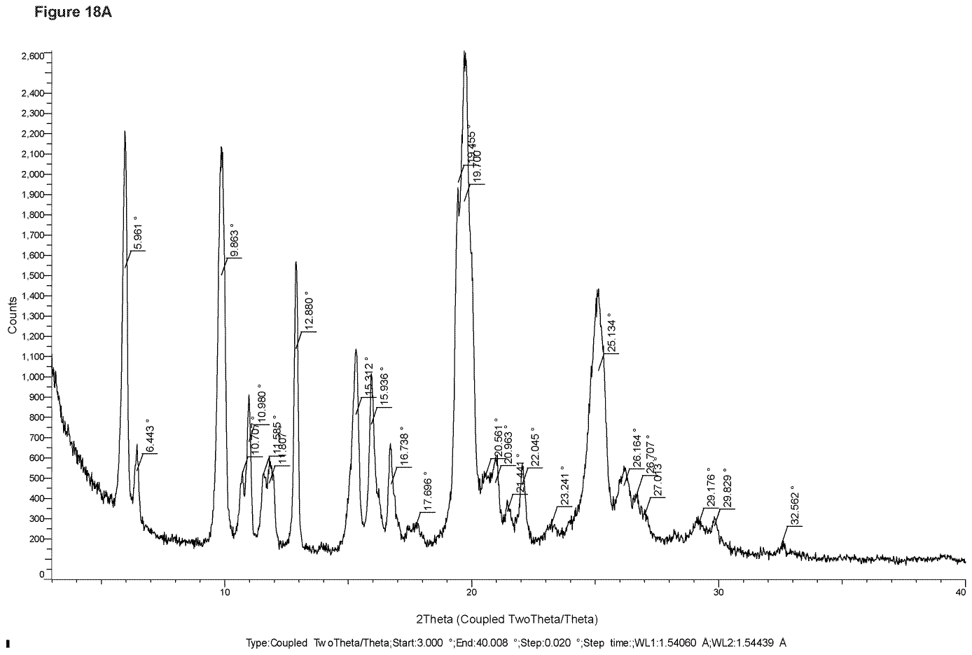

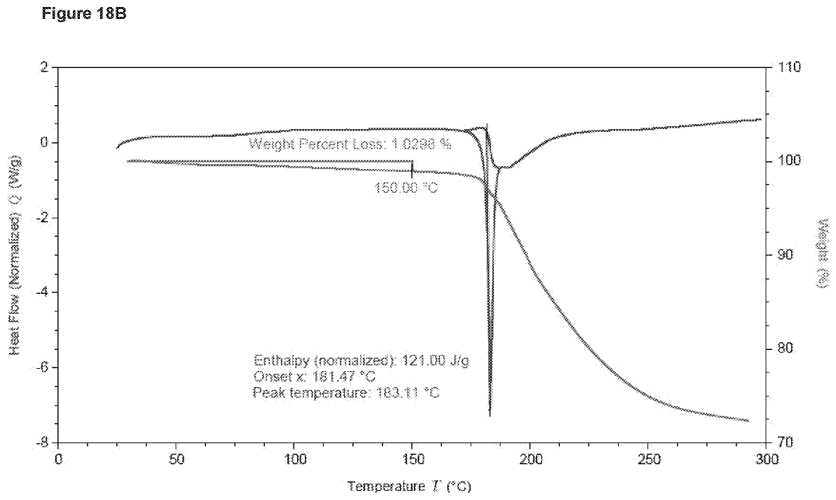

Representative XRPD peaks are tabulated in Table 18. The XRPD patterns and peaks are shown in FIG. 18A. The Thermogravimetric Analysis (TGA) and Differential Scanning Calorimetry Analysis (DSC) thermograms are shown in FIG. 18B.

TABLE 18
Representative XRPD peaks of crystalline
Form I of Fumarate Salt of Compound (I)

| Peak | Angle | Rel. Intensity |
|---|---|---|
| 1 | 5.961° | 68.3% |
| 2 | 6.443° | 13.9% |
| 3 | 9.863° | 75.1% |
| 4 | 10.707° | 19.2% |
| 5 | 10.980° | 28.2% |
| 6 | 11.585° | 19.8% |
| 7 | 11.807° | 17.1% |
| 8 | 12.880° | 55.8% |
| 9 | 15.312° | 37.1% |
| 10 | 15.936° | 33.7% |
| 11 | 16.738° | 16.5% |
| 12 | 17.696° | 3.8% |
| 13 | 19.455° | 100.0% |
| 14 | 19.700° | 94.5% |
| 15 | 20.561° | 17.2% |
| 16 | 20.963° | 15.2% |
| 17 | 21.441° | 7.3% |
| 18 | 22.045° | 15.1% |
| 19 | 23.241° | 3.7% |
| 20 | 25.134° | 45.5% |
| 21 | 26.164° | 13.5% |
| 22 | 26.707° | 12.0% |
| 23 | 27.013° | 6.1% |
| 24 | 29.176° | 5.3% |
| 25 | 29.829° | 6.0% |
| 26 | 32.562° | 3.5% |

### Fumarate Salt Form H

In one embodiment, the present disclosure provides crystalline Form II of fumarate salt of Compound (I), wherein the molar ratio between Compound (I) and fumaric acid is 1:1, which is characterized by XRPD peaks at 5.6°, 15.1°, 16.9°, 19.5°, and 20.0°±0.2 in 2θ.

In another embodiment, the present disclosure provides a crystalline Form II of fumarate salt of Compound (I), wherein the molar ratio between Compound (I) and fumaric acid is 1:1, which is characterized by differential scanning calorimeter (DSC) peak phase transition temperature of 175.1±2° C.

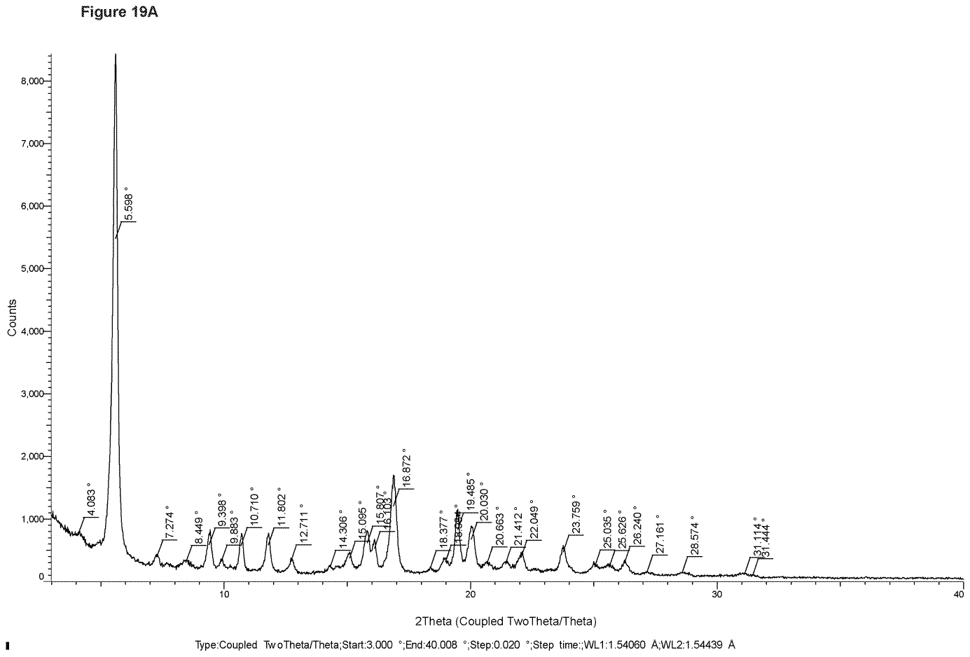

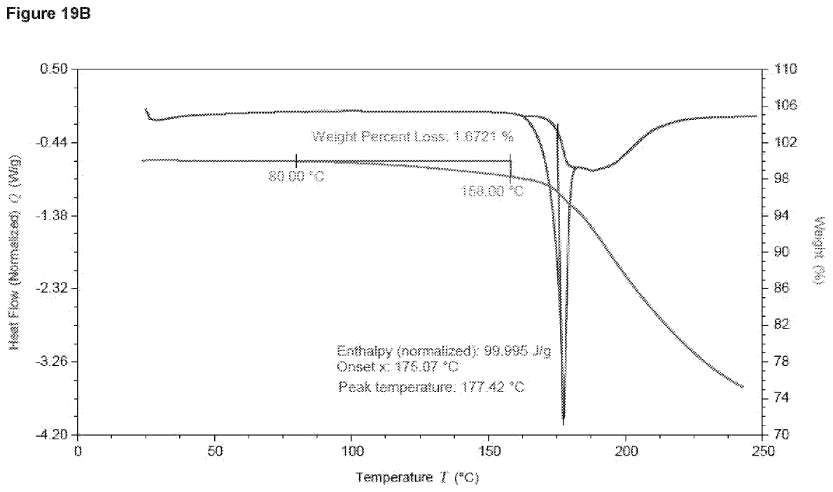

Representative XRPD peaks are tabulated in Table 19. The XRPD patterns and peaks are shown in FIG. 19A. The Thermogravimetric Analysis (TGA) and Differential Scanning Calorimetry Analysis (DSC) thermograms are shown in FIG. 19B.

TABLE 19
Representative XRPD peaks of crystalline
Form II of Fumarate Salt of Compound (I)

| Peak | Angle | Rel. Intensity |
|---|---|---|
| 1 | 4.083° | 1.2% |
| 2 | 5.598° | 100.0% |
| 3 | 7.274° | 2.6% |
| 4 | 8.449° | 1.4% |
| 5 | 9.398° | 7.4% |
| 6 | 9.883° | 2.0% |
| 7 | 10.710° | 7.9% |
| 8 | 11.802° | 8.3% |
| 9 | 12.711° | 3.4% |
| 10 | 14.306° | 1.4% |
| 11 | 15.095° | 4.0% |
| 12 | 15.807° | 8.7% |
| 13 | 16.103° | 6.9% |
| 14 | 16.872° | 20.7% |
| 15 | 18.377° | 1.3% |
| 16 | 18.984° | 2.7% |
| 17 | 19.485° | 12.6% |
| 18 | 20.030° | 9.3% |
| 19 | 20.663° | 1.5% |
| 20 | 21.412° | 1.6% |
| 21 | 22.049° | 3.9% |
| 22 | 23.759° | 6.3% |
| 23 | 25.035° | 1.8% |
| 24 | 25.626° | 1.7% |
| 25 | 26.240° | 3.2% |
| 26 | 27.161° | 0.7% |
| 27 | 28.574° | 0.8% |
| 28 | 31.114° | 0.7% |
| 29 | 31.444° | 0.4% |

### Malate Salt Form I

In one embodiment, the present disclosure provides crystalline Form I of malate salt of Compound (I), wherein the molar ratio between Compound (I) and malic acid is 1:1, which is characterized by XRPD peaks at 7.1, 8.9, 9.9, 13.2, 17.7, and 18.6±0.2 in 2θ.

In another embodiment, the present disclosure provides a crystalline Form I of malate salt of Compound (I), wherein the molar ratio between Compound (I) and malic acid is 1:1, which is characterized by differential scanning calorimeter (DSC) peak phase transition temperature of 156.9±2° C.

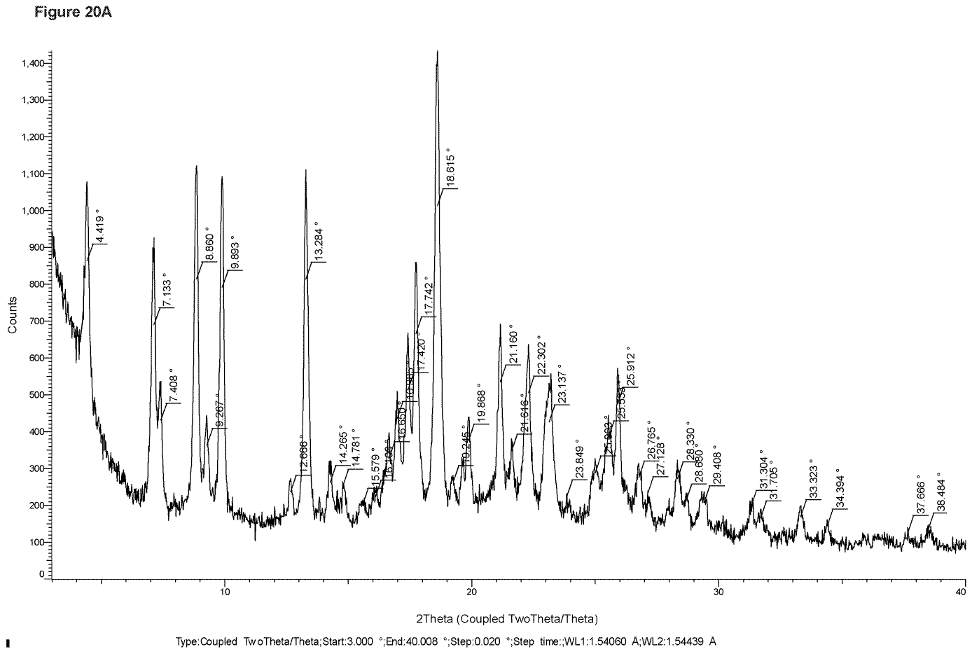

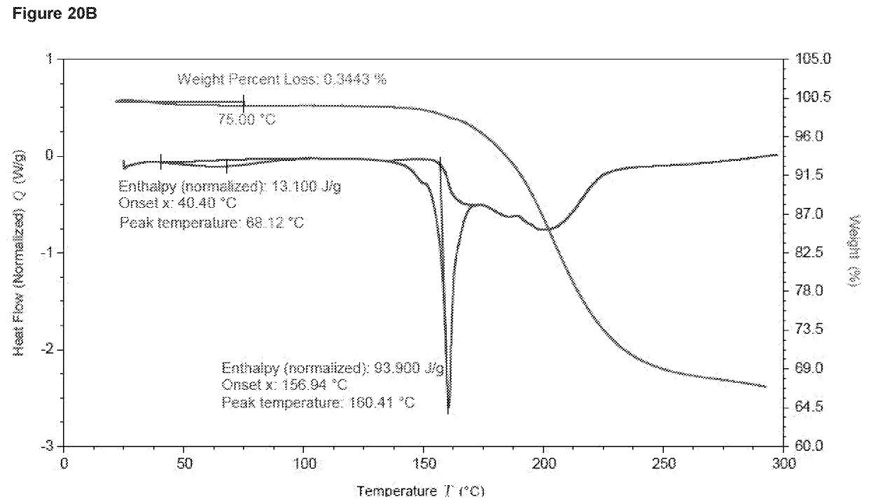

Representative XRPD peaks are tabulated in Table 20. The XRPD patterns and peaks are shown in FIG. 20A. The Thermogravimetric Analysis (TGA) and Differential Scanning Calorimetry Analysis (DSC) thermograms are shown in FIG. 20B.

TABLE 20
Representative XRPD peaks of crystalline
Form I of Succinate Salt of Compound (I)

| Peak | Angle | Rel. Intensity |
|---|---|---|
| 1 | 4.419 | 42.3% |
| 2 | 7.133 | 60.9% |
| 3 | 7.408 | 29.0% |
| 4 | 8.860 | 78.3% |
| 5 | 9.267 | 21.9% |
| 6 | 9.893 | 76.8% |
| 7 | 12.666 | 9.2% |
| 8 | 13.284 | 81.5% |
| 9 | 14.265 | 12.4% |
| 10 | 14.781 | 11.7% |
| 11 | 15.579 | 4.1% |
| 12 | 16.106 | 5.2% |
| 13 | 16.650 | 16.8% |
| 14 | 16.985 | 29.7% |
| 15 | 17.420 | 38.6% |
| 16 | 17.742 | 57.3% |
| 17 | 18.615 | 100.0% |
| 18 | 19.245 | 5.4% |
| 19 | 19.868 | 19.7% |
| 20 | 21.160 | 40.2% |
| 21 | 21.616 | 17.0% |
| 22 | 22.302 | 37.8% |
| 23 | 23.137 | 29.6% |
| 24 | 23.849 | 6.0% |
| 25 | 25.003 | 14.8% |
| 26 | 25.533 | 26.8% |
| 27 | 25.912 | 38.5% |
| 28 | 26.765 | 14.2% |
| 29 | 27.128 | 9.4% |
| 30 | 28.330 | 15.6% |
| 31 | 28.680 | 8.6% |
| 32 | 29.408 | 9.1% |
| 33 | 31.304 | 10.4% |
| 34 | 31.705 | 7.0% |
| 35 | 33.323 | 8.6% |
| 36 | 34.394 | 5.8% |
| 37 | 37.666 | 5.2% |
| 38 | 38.484 | 5.7% |

### Succinate Salt Form I

In one embodiment, the present disclosure provides crystalline Form I of succinate salt of Compound (I), wherein the molar ratio between Compound (I) and succinic acid is 1:1, which is characterized by XRPD peaks at 5.6°, 9.3°, 11.7°, 15.9°, 16.9°, and 20.1°±0.2 in 2θ.

In another embodiment, the present disclosure provides a crystalline Form I of succinate salt of Compound (I), wherein the molar ratio between Compound (I) and succinic acid is 1:1, which is characterized by differential scanning calorimeter (DSC) peak phase transition temperature of 159.7±2° C.

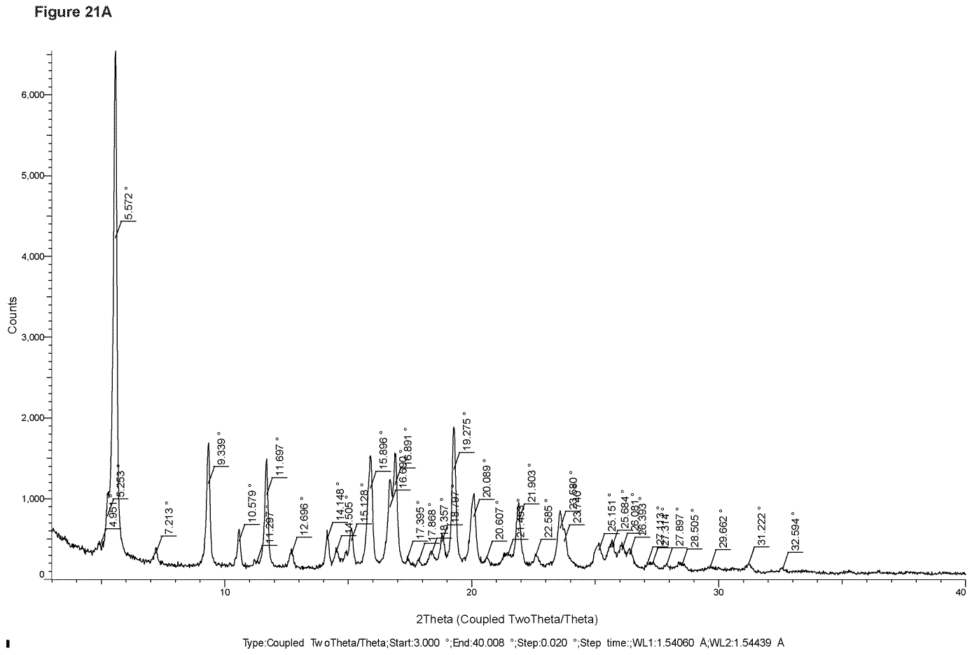

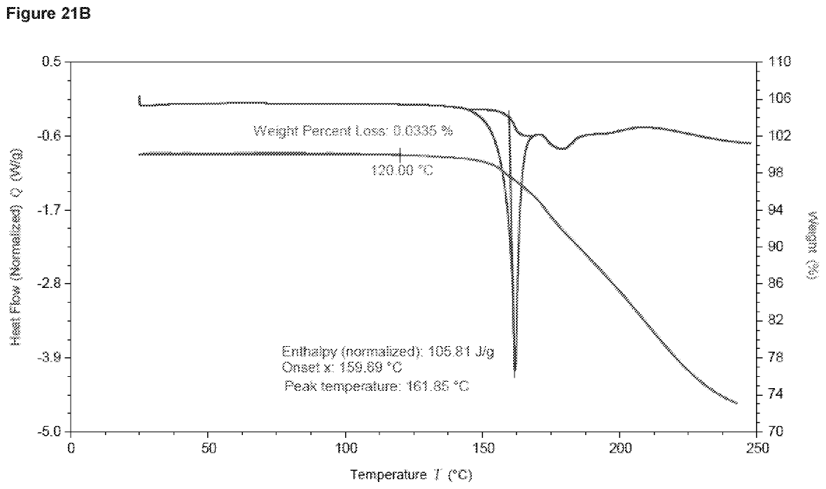

Representative XRPD peaks are tabulated in Table 21. The XRPD patterns and peaks are shown in FIG. 21A. The Thermogravimetric Analysis (TGA) and Differential Scanning Calorimetry Analysis (DSC) thermograms are shown in FIG. 21B.

TABLE 21
Representative XRPD peaks of crystalline
Form I of Succinate Salt of Compound (I)

| Peak | Angle | Rel. Intensity |
|---|---|---|
| 1 | 4.951° | 2.5% |
| 2 | 5.253° | 12.3% |
| 3 | 5.572° | 100.0% |
| 4 | 7.213° | 3.2% |
| 5 | 9.339° | 25.8% |
| 6 | 10.579° | 7.7% |
| 7 | 11.297° | 1.1% |
| 8 | 11.697° | 22.4% |
| 9 | 12.696° | 4.1% |
| 10 | 14.148° | 8.6% |
| 11 | 14.505° | 4.2% |
| 12 | 15.128° | 7.6% |
| 13 | 15.896° | 23.9% |
| 14 | 16.690° | 18.0% |
| 15 | 16.891° | 25.0% |
| 16 | 17.395° | 2.2% |
| 17 | 17.868° | 1.5% |
| 18 | 18.357° | 2.9% |
| 19 | 18.797° | 6.9% |
| 20 | 19.275° | 29.6% |
| 21 | 20.089° | 14.9% |
| 22 | 20.607° | 1.9% |
| 23 | 21.453° | 2.6% |
| 24 | 21.903° | 13.6% |
| 25 | 22.585° | 2.7% |
| 26 | 23.580° | 11.9% |
| 27 | 23.740° | 7.7% |
| 28 | 25.151° | 5.2% |
| 29 | 25.684° | 6.2% |
| 30 | 26.081° | 5.3% |
| 31 | 26.393° | 4.0% |
| 32 | 27.113° | 1.3% |
| 33 | 27.314° | 1.1% |
| 34 | 27.897° | 1.2% |
| 35 | 28.505° | 1.2% |
| 36 | 29.662° | 1.1% |
| 37 | 31.222° | 2.4% |
| 38 | 32.594° | 0.9% |

### Methods of Treatment

Certain compounds of the present invention are selective inhibitors of CDK2, CDK4, and/or CDK6, and are therefore useful in the treatment of a disease or disorder characterised by abnormal cell proliferation that can be inhibited by a reduced activity of CDK-cyclin complexes encompassing CDK2, CDK4, and/or CDK6.

In certain embodiments, compounds of the invention selectively inhibit CDK4/6 over CDK2, with a ratio of IC₅₀ values for the latter (CDK2) against the former (CDK4/6) of at least about 10, 20, 50, 100, 200, 300, 400, 500, 800, 1,000, 2,000 or more.

In certain embodiments, compounds of the invention selectively inhibit CDK4 over CDK6, with a ratio of IC₅₀ values for the latter (CDK6) against the former (CDK4) of at least about 2, 3, 4, 5, 6, 7, 8, 9, 10, 15, 20, 50 or more.

In certain embodiments, compounds of the invention selectively inhibit CDK2 over CDK4, with a ratio of IC₅₀ values for the latter (CDK4) against the former (CDK2) of at least about 2, 5, 10, 15, 20, 40, 50, 60, 80, 100 or more.

In certain embodiments, compounds of the invention inhibits CDK2/4/6 with similar IC₅₀ values, e.g., IC₅₀ values within 10-, 5-, 3-, or 2-fold. Such compounds of the invention are useful for treating cancers with cyclin D1 or E1 or E2 amplification or enhanced expression.

CDK2 is the catalytic subunit of the CDK-cyclin complex whose activity is restricted to the G1-S phase of the cell cycle, where cells make proteins necessary for mitosis and replicate their DNA. CDK2 is complexed with cyclin E or A. Cyclin E binds G1 phase CDK2, which is required for G1 to S phase transition. On the other hand, CDK2 binding with Cyclin A is required to progress through the S phase.

Although CDK2 is mostly dispensable in the cell cycle of normally functioning cells, it is critical to the abnormal growth processes of cancer cells. Overexpression of cyclin E occurs in many tumor cells, causing the cells to become dependent on CDK2 and cyclin E. Abnormal cyclin E activity is observed in breast, lung, colorectal, gastric, and bone cancers, as well as in leukemia and lymphoma. Likewise, abnormal expression of cyclin A2 is associated with chromosomal instability and tumor proliferation, while inhibition leads to decreased tumor growth. Therefore, CDK2 and its cyclin binding partners represent possible therapeutic targets for new cancer therapeutics. Pre-clinical models have shown preliminary success in limiting tumor growth, and have also been observed to reduce side effects of current chemotherapy drugs.

For example, Caldon et al. (Mol Cancer Ther 11(7):1488-1499, 2012) reported that Cyclin E2 is included in several gene signatures that predict disease progression in either tamoxifen-resistant or metastatic breast cancer, and high expression of CycE2 was characteristic of the luminal B and HER2 subtypes of breast cancer and was strongly predictive of shorter distant metastasis-free survival following endocrine therapy. Further, tamoxifen-resistant (MCF-7 TAMR) breast cancer cells overexpressed cyclin E2; and expression of either cyclin E1 or E2 in T-47D breast cancer cells conferred acute antiestrogen resistance, suggesting that cyclin E overexpression contributes to the antiestrogen resistance of tamoxifen-resistant cells. Proliferation of tamoxifen-resistant cells was inhibited by RNAi-mediated knockdown of cyclin E1, cyclin E2, or CDK2. Besides, ectopic expression of cyclin E1 or E2 also reduced sensitivity to CDK4, but not CDK2, inhibition. Furthermore, CDK2 inhibition of E-cyclin overexpressing cells and tamoxifen-resistant cells restored sensitivity to tamoxifen or CDK4 inhibition.

These data demonstrate that Cyclin E2 overexpression is a potential mechanism of resistance to both endocrine therapy and CDK4 inhibition, and CDK2 inhibitors may in turn overcome such resistance, and may be beneficial as a component of combination therapies in endocrine-resistant disease as they effectively inhibit cyclin E1 and E2 overexpressing cells and enhance the efficacy of other therapeutics. Likewise, the subject compounds with potent inhibitory activities against both CDK2 and CDK4 are expected to be effective against cancer cells that are both non-resistant and resistant to endocrine therapy or CDK4 inhibition.

Thus in certain embodiments, the compounds of the invention may have potent inhibitory effects against both CDK2 and CDK4 (e.g., independently <10 nM, <5 nM, <1 nM level of IC₅₀ values), and thus are effective to treat tamoxifen-resistant or metastatic breast cancers, such as tamoxifen-resistant or metastatic breast cancers with CycE overexpression.

IC₅₀ values of the compounds of the invention against CDK2/4/6 can be measured using, for example, the methods described in Examples 1-3 (incorporated herein by reference).

In particular, the compounds of the present invention are useful in the treatment of cancer. In other embodiments, the compounds of the present invention are useful in the treatment of chronic inflammation diseases such as arthritis and cystic fibrosis.

Thus in one aspect, the present invention provides a method of treating cancer, in particular the cancers described herein, in a mammal, comprising administering to a mammal in need of such treatment an effective amount of a compound of the present invention.

In a related aspect, the invention is directed to a use of a compound of the present invention in the manufacture of a medicament for treating cancer, in particular, the cancers described herein.

In another related aspect, the compounds of the present invention can be used in the manufacture of a medicament for the treatment of cancer, in particular, the cancers described herein.

In another related aspect, the invention provides a compound of the present invention for use in treating cancer, in particular, the cancers described herein.

According to any of the above related aspects of the invention, CDK4 and CDK6 may modulate their effects on the cell cycle partly through pRb phosphorylation. Thus, certain compounds of the present invention may inhibit pRb phosphorylation through inhibiting CDK4/6 activity, and thus inhibiting cell proliferation and/or tumor growth, in any cancer type where the cells are proliferating and contain a functional, intact Rb1 gene that encodes pRb.

Thus in certain embodiments, the compounds of the invention are useful in the treatment of pRb⁺ cancers, such as colorectal cancer, breast cancer, lung cancer, prostate cancer, chronic myeloid leukemia, acute myeloid leukemia (Fry et al., Mol. Cancer Ther. 3(11):1427, 2004), mantel cell lymphoma (Marzec et al., Blood 108(5):1744, 2006), ovarian cancer (Kim et al., Cancer Research 54:605, 1994), pancreatic cancer (Schutte et al., Cancer Research 57:3126, 1997), malignant melanoma and metastatic malignant melanoma (Maelandsmo et al., British Journal of Cancer 73:909, 1996) in mammals. The compounds of the invention are also expected to be useful in the treatment of rhabdomyosarcoma (Saab et al., Mol. Cancer. Ther. 5(5):1299, 2006) and multiple myeloma (Baughn et al., Cancer Res. 66(15):7661, 2006), including relapsed refractory multiple myeloma, in mammals (e.g., human).

Meanwhile, Zhang et al. (Nature dx.doi.org/10.1038/nature25015, 2017) reported that inhibition of CDK4/6 in vivo may lead to decreased phosphorylation and therefore increased degradation of Cullin 3^{SPOP} E3 ligase (by APC/C^{cdhl}), which in turn leads to increased PD-L1 levels on tumor cell surface, and reduced numbers of tumor-infiltrating lymphocytes (TILs) in mouse tumors and in primary human prostate cancer specimens. In other words, inhibition of CDK4/6 in vivo elevates PD-L1 protein levels, and contributes to increased resistance to immune checkpoint therapy targeting PD-1 (programmed cell death protein 1) and PD-L1 (ligand for PD-1). On the other hand, combining CDK4/6 inhibitor treatment with anti-PD-1 immunotherapy enhances tumor regression, and dramatically improves overall survival rates in mouse tumor models.

Thus in certain embodiments, the compounds of the invention can be used in combination with PD-1/PD-L1 immune checkpoint inhibitors to enhance therapeutic efficacy for human cancers.

PD-1 and PD-L1 inhibitors that can be used with the compounds of the invention are known in the art. PD-1 inhibitors include monoclonal antibodies or antigen binding fragment thereof specific for PD-1. Exemplary PD-1 inhibitors include Pembrolizumab (Keytruda), Nivolumab (Opdivo), and Cemiplimab (Libtayo). PD-L1 inhibitors include monoclonal antibodies or antigen binding fragment thereof specific for PD-L1. Exemplary PD-L1 inhibitors include Atezolizumab (Tecentriq), Avelumab (Bavencio), and Durvalumab (Imfinzi).

Additional immune checkpoint inhibitor that may be used with the compounds of the invention for enhancing therapeutic efficacy for human cancers include monoclonal antibodies or antigen binding fragments thereof specific for CTLA-4 such as Ipilimumab (Yervoy).

Further immune checkpoint inhibitor that may be used with the compounds of the invention for enhancing therapeutic efficacy for human cancers include bispecific monoclonal antibodies or antigen binding fragments thereof specific for PD-1 and PD-L1, or combination of monoclonal antibodies or antigen binding fragments thereof specific for PD-1 and PD-L1, or PD-1 and CTLA-4, etc.

In certain embodiments, the compounds of the invention can be used in combination with Tyr kinase inhibitor, e.g., receptor Tyr kinase (RTK) inhibitors, to enhance therapeutic efficacy for human cancers. Exemplary Tyr kinase inhibitors include ALK inhibitors (such as Crizotinib, Ceritinib, Alectinib, Brigatinib), Bcr-Abl inhibitors (such as Bosutinib, Dasatinib, Imatinib, Nilotinib, Ponatinib), BTK inhibitor (such as Ibrutinib), c-Met inhibitor (such as Crizotinib, Cabozantinib), EGFR inhibitor (such as Gefitinib, Erlotinib, Lapatinib, Vandetanib, Afatinib, Osimertinib), JAK inhibitor (such as Ruxolitinib, Tofacitinib), MEK1/2 inhibitor (such as Trametinib), PDGFR inhibitor (such as Axitinib, Gefitinib, Imatinib, Lenvatinib, Nintedanib, Pazopanib, Regorafenib, Sorafenib, Sunitinib), RET inhibitor (such as Vandetanib), Src family kinase inhibitors (such as Bosutinib, Dasatinib, Ponatinib, Vandetanib), and VEGFR family inhibitors (such as Axitinib, Lenvatinib, Nintedanib, Regorafenib, Pazopanib, Sorafenib, Sunitinib).

Additional suitable kinase inhibitors that can be used in combination with the subject compounds, as well as the treatable cancer indications, are described in Bhullar et al., Molecular Cancer 17:48, 2018 (incorporated herein by reference in its entirety).

Further additional RTK inhibitors include monoclonal antibodies and antigen-binding fragments thereof, including the anti-EGFR mAB such as cetuximab (effective in treating, e.g., lung, colorectal, and head and neck cancer), and the anti-HER2 mAb such as trastuzumab (effective to treat, for example, breast cancer).

In certain embodiments, the compounds of the invention can be used in combination with an antagonist of hormonal receptor signaling, such as the ones described before for breast cancer treatment.

Cancers treatable with the compounds of the invention include: Non-Hodgkin's lymphoma; malignant mesothelioma; non-small cell lung cancer; cholangiocarcinoma; soft tissue sarcoma; glioblastoma; (recurrent) brain tumor; brain metastases secondary to hormone receptor positive breast cancer, non-small cell lung cancer, melanoma (including melanoma positive for cyclin D1 expression); (recurrent or persistent) endometrial cancer; (recurrent or metastatic) Head and Neck Squamous Cell Carcinoma (HNSCC); hepatocellular carcinoma; esophageal squamous cell carcinoma (SCC); esophageal adenocarcinoma (ADC); renal cell carcinoma, and urothelial cancer.

In certain embodiments, the treatable cancers include: carcinoma of the bladder, breast, colon, kidney, epidermis, liver, lung (including SCLC and NSCLC), esophagus, gall bladder, ovary, pancreas, stomach, cervix, thyroid, nose, head and neck, prostate, or skin; a hematopoietic tumor of lymphoid lineage; a hematopoietic tumor of myeloid lineage; thyroid follicular cancer; a tumor of mesenchymal origin; a tumor of the central or peripheral nervous system; melanoma; familial melanoma; seminoma; teratocarcinoma; osteosarcoma; xeroderma pigmentosum; keratoctanthoma; thyroid follicular cancer; Kaposi's sarcoma, squamous cancer, sarcoma; or a tumor of mesenchymal origin.

In certain embodiments, the hematopoietic tumor of lymphoid lineage is leukemia, acute lymphocytic leukemia, chronic lymphocytic leukemia, B-cell lymphoma, T-cell lymphoma, multiple myeloma, Hodgkin's lymphoma, non-Hodgkin's lymphoma, hairy cell lymphoma, or Burkett's lymphoma.

In certain embodiments, the tumor of the central or peripheral nervous system is astrocytoma, neuroblastoma, glioma or schwannoma.

In certain embodiments, the cancer is small cell lung cancer, non-small cell lung cancer, pancreatic cancer, breast cancer, glioblastoma multiforme, T cell ALL and mantle cell lymphoma.

In certain embodiments, the cancer is selected from the group consisting of: colorectal cancer, mantel cell lymphoma, breast cancer (including advanced or metastatic or recurrent breast cancer), pancreatic cancer, ovarian cancer, glioblastoma, acute myeloid leukemia, and lung cancer, especially NSCLC.

In certain embodiments, the cancer is NSCLC, pancreatic cancer, ovarian cancer or metastatic breast cancer, and the treatment comprising administering to a mammal in need thereof a therapeutically effective combination of a compound of the present invention and gemcitabine HCl.

In certain embodiments, the cancer is NSCLC, pancreatic cancer, ovarian cancer or metastatic breast cancer, wherein the medicament comprising the compound of the present invention also comprises gemcitabine HCl, or is to be administered simultaneously, separately or sequentially with gemcitabine HCl.

In certain embodiments, the compounds of the present invention can be used in combination with other agents for the treatment of NSCLC, pancreatic cancer, ovarian cancer and metastatic breast cancer. For example, the compound of the present invention may be used in simultaneous, separate or sequential combination with gemcitabine HCl in the treatment of NSCLC, pancreatic cancer, ovarian cancer or metastatic breast cancer.

In certain embodiments, the cancer is selected from the group consisting of colorectal cancer, glioblastoma, acute myeloid leukemia and lung cancer.

In certain embodiments, the cancer is glioblastoma or astrocytoma, and the treatment utilizes a therapeutically effective combination of a compound of the invention and temozolomide. The compound of the invention may be administered simultaneously, separately or sequentially with temozolomide.

### Breast Cancer Treatment

In certain embodiments, the compounds of the invention can be used to treat breast cancer.

Breast cancer presents a significant health burden worldwide, and it alone accounted for ~7% of all US cancer-related deaths in 2016. Of all breast cancers, about 75% are diagnosed as hormone receptor-positive (HR⁺) breast cancer, which expresses the estrogen receptor (ER) and/or the progesterone receptor (PgR), and is typically dependent on the ER signaling pathway for growth and survival. That is, the HR⁺ breast cancers harness the biological functions of the ER pathway to promote breast cancer growth, development, and progression. Meanwhile, the reliance of HR⁺ breast cancer on ER signaling made such breast cancer a therapeutic target for endocrine therapy agents that target the estrogen signaling pathway, such as aromatase inhibitors (AIs; including letrozole, anastrozole, and exemestane), selective ER modulators (tamoxifen), and selective ER down-regulators (fulvestrant), etc.

Although endocrine therapy makes up the treatment backbone for HR⁺ breast cancer, the efficacy of endocrine therapy is limited by high rates of both pre-existing de novo resistance, and resistance acquired during treatment, due to the presence of alternative survival or “escape” pathway. The ER pathway and many of the known escape pathways act through the cyclin D-CDK4/6-inhibitor of CDK4 (INK4)-retinoblastoma (Rb) pathway to promote tumor growth. As such, targeting both the ER and the cyclin D-CDK4/6-INK4-Rb pathways in combination usually lead to a more extensive inhibition of tumor growth and prevent the activation of escape pathways, precluding the development of endocrine therapy resistance. See Sammons et al., Current Cancer Drug Targets 17:637-649, 2017.

Thus in certain embodiments, the breast cancer is a pRb⁺ breast cancer. In certain embodiments, the breast cancer is a hormone receptor (HR)-positive (e.g., estrogen receptor positive (ER⁺), progesterone receptor positive (PR⁺), or ER⁺PR⁺), HER2/neu-negative cancer, including HR⁺HER2⁻ or ER⁺HER2⁻, advanced or metastatic or recurrent breast cancer. In certain embodiments, the HR⁺HER2⁻ or ER⁺HER2⁻ advanced or metastatic or recurrent breast cancer is in an adult woman, or a postmenopausal woman.

In certain embodiments, the compounds of the invention is either used alone, or used with an aromatase inhibitor (that inhibits estrogen production), to treat HR-positive, HER2⁻ negative advanced or metastatic or recurrent breast cancer. In certain embodiments, the aromatase inhibitor temporarily inactivate aromatase (such as anastrozole (ARIMIDEX®) and letrozole (FEMARA®)). In certain embodiments, the aromatase inhibitor permanently inactivate aromatase (such as exemestane (AROMASIN©)).

In certain embodiments, the compound(s) of the invention is used with a compound that interferes with estrogen's ability to stimulate the growth of breast cancer cells, such as a Selective Estrogen Receptor Modulator (SERM) that binds to the estrogen receptor to prevent estrogen binding, such as tamoxifen (NOLVADEX®) and toremifene (FARESTON®). Tamoxifen has been used for more than 30 years to treat HR⁺ breast cancer.

In certain embodiments, the compound(s) of the invention is used with a pure antiestrogen with no estrogen agonist activity, such as fulvestrant (FASLODEX®).

In certain embodiments, the HR-positive, HER2⁻ negative advanced or metastatic or recurrent breast cancer is in a postmenopausal woman. In certain embodiments, the HR-positive, HER2⁻ negative advanced or metastatic or recurrent breast cancer has progressed after taking therapy that alters a patient's hormones (e.g., estrogen and/or progesterone), or has worsened after treatment with another hormone therapy.

In certain embodiments, the compound(s) of the invention is used in a patient undergoing ovarian ablation, or has received ovarian ablation. In certain embodiments, the ovarian ablation is through oophorectomy or radiation treatment.

In certain embodiments, the compound(s) of the invention is used with a compound that temporarily suppresses ovarian function (e.g., estrogen and/or progesterone production). Such compound includes gonadotropin-releasing hormone (GnRH) agonists or luteinizing hormone-releasing hormone (LH-RH) agonists, including goserelin (ZOLADEX®) and leuprolide (LUPRON®).

In certain embodiments, the compound(s) of the invention is used with a compound that inhibits CYP3A4, such as ritonavir, indinavir, nelfinavir, saquinavir, clarithromycin, telithromycin, chloramphenicol, ketoconazole, itraconazole, posaconazole, voriconazole, nefazodone, cobicistat, amiodarone, aprepitant, verapamil, diltiazem, erythromycin, fluconazole, miconazole, bergamottin, cimetidine, ciprofloxacin, cyclosporine, donedarone, fluvoxamine, imatinib, Valerian, buprenorphine, cafestol, cilostazol, fosaprepitant, gabapentin, lomitapide, orphenadrine, ranitidine, ranolazine, tacrolimus, ticagrelor, valproic acid, amlodipine, cannabidiol, dithiocarbamate, mifepristone, norfloxacin, delavirdine, gestodene, mibefradil, star fruit, milk thistle, niacinamide, Ginkgo biloba, piperine, isoniazid, and quercetin.

In certain embodiments, the compound(s) of the invention is used with an inhibitor of IGF-1/IGF-2, such as a monoclonal antibody or an antigen-binding fragment thereof against IGF-1/IGF-2. Exemplary antibodies include xentuzumab, a humanized IgG1 mAb.

In certain embodiments, the compound(s) of the invention is used with a compound that inhibits PI3K. It is believed that inhibition of PI3K reduces the levels of cyclin D1 and other G1-S cyclins, abolishes pRb phosphorylation, and inhibits activation of S-phase transcriptional programs. Representative PI3K inhibitors for use with the compounds of the invention includes idelalisib, copanlisib, duvelisib, taselisib, perifosine, buparlisib, alpelisib, umbralisib, copanlisib, dactolisib, and voxtalisib.

In certain embodiments, the mammal to be treated is a human, such as an adult woman having breast cancer (e.g., postmanupausal woman or adult woman having hormone receptor (HR)-positive, human epidermal growth factor receptor 2 (HER2)-negative advanced or metastatic or recurrent breast cancer that has progressed after taking therapy that alters a patient's hormones).

Additionally, certain compounds of the present invention exhibit the advantageous property that they are able to cross the blood-brain barrier. Such compounds are therefore able to penetrate the brain and are thus useful in the treatment of primary and metastatic brain tumors where the cells are proliferating and contain a functional, intact Rb1 gene. Examples of such pRb⁺ brain tumors include glioblastoma, as well as medulloblastoma and astrocytoma (Lee et al., Science 235:1394, 1987).

Temozolomide is a cytotoxic, DNA alkylating agent used for the treatment of brain tumors including glioblastoma and astrocytoma (Friedman et al., Clin. Cancer Res. 6(7):2585-2597, 2000) including brain metastases from melanoma, breast cancer and NSCLC (Siena et al., Annals of Oncology, doi:10.1093/annonc/mdp343, 2009). Temozolomide interacts with DNA causing chemical modification/damage (Marchesi et al., Pharmacol. Res. 56(4):275-287, 2007). Thus, in some embodiments, the compounds of the present invention can be used in combination with temozolomide for the treatment of primary and metastatic pRb⁺ brain tumors such as glioblastoma and astrocytoma, for example, where such metastases are derived from melanoma, breast cancer or NSCLC.

### Pharmaceutical Compositions

The invention provides pharmaceutical compositions which comprise any one of the compounds described herein, or a pharmaceutically acceptable salt thereof, and one or more pharmaceutically acceptable carriers or excipients.

“Pharmaceutically acceptable excipient” and “pharmaceutically acceptable carrier” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical carriers and excipients are suitable for use with disclosed compounds.

These compositions optionally further comprise one or more additional therapeutic agents. Alternatively, a compound of the invention may be administered to a patient in need thereof in combination with the administration of one or more other therapeutic regimens (e.g. Gleevec or other kinase inhibitors, interferon, bone marrow transplant, farnesyl transferase inhibitors, bisphosphonates, thalidomide, cancer vaccines, hormonal therapy, antibodies, radiation, etc). For example, additional therapeutic agents for conjoint administration or inclusion in a pharmaceutical composition with a compound of this invention may be another one or more anticancer agents.

As described herein, the compositions of the present invention comprise a compound of the invention together with a pharmaceutically acceptable carrier, which, as used herein, includes any and all solvents, diluents, or other vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition.

### Formulations

This invention also encompasses a class of compositions comprising the active compounds of this invention in association with one or more pharmaceutically-acceptable carriers and/or diluents and/or adjuvants (collectively referred to herein as “carrier” materials) and, if desired, other active ingredients.

In certain embodiments, the invention provides a pharmaceutical formulation for treating cancer, in particular the cancers described herein, comprising a compound of the present invention or a pharmaceutically acceptable salt thereof together with a pharmaceutically acceptable carrier.

In certain embodiments, the invention provides a pharmaceutical formulation for treating a cancer selected from the group consisting of colorectal cancer, mantel cell lymphoma, breast cancer (including ER⁺HER2⁻ advanced or metastatic or recurrent breast cancer in an adult woman, or a postmenopausal woman), glioblastoma, acute myeloid leukemia and lung cancer, especially NSCLC, comprising a compound of the present invention or a pharmaceutically acceptable salt thereof together with a pharmaceutically acceptable carrier.

In certain embodiments, the invention provides a pharmaceutical formulation for treating glioblastoma or astrocytoma, comprising a compound of the invention and temozolomide, together with a pharmaceutically acceptable carrier.

In certain embodiments, the invention also provides a pharmaceutical formulation, comprising a compound of the invention or a pharmaceutically acceptable salt thereof and temozolomide, together with a pharmaceutically acceptable carrier, diluent, or excipient.

In certain embodiments, the invention provides a pharmaceutical formulation for treating NSCLC, pancreatic cancer, ovarian cancer or metastatic breast cancer (including ER⁺HER2⁻ advanced or metastatic or recurrent breast cancer in an adult woman, or a postmenopausal woman), comprising a compound of the invention and gemcitabine HCl, together with a pharmaceutically acceptable carrier.

In certain embodiments, the invention also provides a pharmaceutical formulation, comprising a compound of the invention or a pharmaceutically acceptable salt thereof and gemcitabine HCl, together with a pharmaceutically acceptable carrier, diluent, or excipient.

The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly, intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient.

Examples of such dosage units are tablets or capsules. For example, a suitable daily dose for a human or other mammal may vary depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. As mentioned previously, the daily dose can be given in one administration or may be divided between 2, 3, 4 or more administrations.

For therapeutic purposes, the active compounds of this invention are ordinarily combined with one or more adjuvants, excipients or carriers appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In the case of skin conditions, it may be preferable to apply a topical preparation of compounds of this invention to the affected area two to four times a day. Formulations suitable for topical administration include liquid or semi-liquid preparations suitable for penetration through the skin (e.g., liniments, lotions, ointments, creams, or pastes) and drops suitable for administration to the eye, ear, or nose. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

The compounds of this invention can also be administered by a transdermal device. Preferably transdermal administration will be accomplished using a patch either of the reservoir and porous membrane type or of a solid matrix variety. In either case, the active agent is delivered—continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The oily phase of the emulsions of this invention may be constituted from known ingredients in a known manner.

While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required.

Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients.

The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents. Pharmaceutical compositions of this invention comprise a compound of the formulas described herein or a pharmaceutically acceptable salt thereof; an additional agent selected from a kinase inhibitory agent (small molecule, polypeptide, antibody, etc.), an immunosuppressant, an anticancer agent, an anti-viral agent, antiinflammatory agent, antifungal agent, antibiotic, or an anti-vascular hyperproliferation compound; and any pharmaceutically acceptable carrier, adjuvant or vehicle.

Alternate compositions of this invention comprise a compound of the formulae described herein or a pharmaceutically acceptable salt thereof; and a pharmaceutically acceptable carrier, adjuvant or vehicle. Such compositions may optionally comprise one or more additional therapeutic agents, including, for example, kinase inhibitory agents (small molecule, polypeptide, antibody, etc.), immunosuppressants, anti-cancer agents, anti-viral agents, antiinflammatory agents, antifungal agents, antibiotics, or anti-vascular hyperproliferation compounds.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound. Pharmaceutically acceptable carriers, adjuvants and vehicles that may be used in the pharmaceutical compositions of this invention include, but are not limited to, ion exchangers, alumina, aluminum stearate, lecithin, selfemulsifying drug delivery systems (SEDDS) such as d-atocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as u-, P-, and y-cyclodextrin, or chemically modified derivatives such as hydroxyalkylcyclodextrins, including 2 and 3-hydroxypropyl-cyclodextrins, or other solubilized derivatives may also be advantageously used to enhance delivery of compounds of the formulae described herein.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents.

If desired, certain sweetening, flavoring and/or coloring agents may be added. The pharmaceutical compositions may comprise formulations utilizing liposome or microencapsulation techniques, various examples of which are known in the art.

The pharmaceutical compositions may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents, examples of which are also well known in the art.

### Treatment Kits

One aspect of the present invention relates to a kit for conveniently and effectively carrying out the methods or uses in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope. Indeed, various modifications of the invention, and many further embodiments thereof, in addition to those shown and described herein, will become apparent to those skilled in the art upon review of this document, including the examples which follow and the references to the scientific and patent literature cited herein.

The contents of the cited references are incorporated herein by reference to help illustrate the state of the art.

In addition, for purposes of this invention, the chemical elements are identified in accordance with the Periodic Table of the Elements, CAS version, Handbook of Chemistry and Physics, 75^{th} Ed., inside cover. Additionally, general principles of organic chemistry, as well as specific functional moieties and reactivity, are described in “Organic Chemistry,” Thomas Sorrell, University Science Books, Sausalito: 1999, and “Organic Chemistry,” Morrison & Boyd (3d Ed), the entire contents of both of which are incorporated herein by reference.

### EXPERIMENTAL

### Abbreviations

| Abbreviation | Solvent | Abbreviation | Solvent |
|---|---|---|---|
| ACN | Acetonitrile | BA | Benzyl Alcohol |
| DCM | Dichloromethane | DEE | Diethyl ether |
| Tol | Toluene | IBA | Isobutanol |
| Hept | n-Heptane | DMSO | Dimethylsulfoxide |
| EA | Ethyl Acetate | FA | Formic Acid |
| IPA | 2-Propanol | IPOAc/IPAc | Isopropyl acetate |
| EtOH | Ethanol | MCH | Methylcyclohexane |
| MEK | 2-Butanone | MeOAc | Methyl Acetate |
| MeOH | Methanol | MIBK | 4-Methyl-2-pentanone |
| MTBE | tert-Butyl | NMP | N-Methyl Pyrrolidone |
| | Methyl Ether | | |
| 1-PA | 1-Propanol | TFA | Trifluoroacetic Acid |
| IPA | 2-propanol | H₂O | Water |
| IPAC | Isopropyl acetate | THF | Tetrahydrofuran |

Instruments

| Full Name | Abbreviation |
|---|---|
| Differential scanning calorimetry | DSC |
| Dynamic Vapor Sorption | DVS |
| High Performance Liquid Chromatography | HPLC |
| Nuclear Magnetic Resonance | NMR |
| Polarized Light Microscopy | PLM |
| X-ray Powder Diffraction | XRPD |
| Thermogravimetric Analysis | TGA |

Units

| Full Name | Abbreviation |
|---|---|
| Celsius | C. |
| Degrees | ° |
| Equivalents | eq. |
| Gram | g |
| Hour | H |
| Hertz (S⁻¹) | Hz |
| Milligrams | mg |
| Milliliters | mL |
| Minute | min |
| Relative Humidity | RH |
| Room temperature | RT |
| Second | sec |
| volume | vol. |
| Volume ratio | v/v |
| Weight | wt. |
| Weight Percentage | wt. % |

### Analysis Conditions

### X-Ray Powder Diffraction (XRPD)

XRPD patterns were identified with an X-ray diffractometer (Bruker D8 advance). The system was equipped with LynxEye detector. Samples were scanned from 3 to 40° 2θ, at a step size 0.02° 2θ. The tube voltage and current were 40 KV and 40 mA, respectively.

### Differential Scanning Calorimeter (DSC)

DSC was performed using a Discovery DSC 250 (TA Instruments, US). The sample was placed into an aluminum pin-hole hermetic pan and the weight was accurately recorded. The sample was heated at a rate of 10° C./min from 25° C. to the final temperature.

### Thermogravimetric Analysis (TGA)

TGA was carried out on a Discovery TGA 55 (TA Instruments, US). The sample was placed into an open tared aluminum pan, automatically weighed, and inserted into the TGA furnace. The sample was heated at a rate of 10° C./min from ambient temperature to the final temperature.

### Dynamic Vapor Sorption (DVS)

Moisture sorption/desorption data was collected on a DVS Intrinsic PLUS (SMS, UK). The sample was placed into a tared sample chamber and automatically weighed. The sample was dried at 40° C./0% RH until the dm/dt was less than 0.002% and cooled to 25° C. The instrument parameters were set as below.
Sample temperature: 25° C.
Min time per step: 30 min
Max time per step: 120 min
Cycle: Full cycle
Adsorption: 0, 10, 20, 30, 40, 50, 60, 70, 80, 90
Desorption: 80, 70, 60, 50, 40, 30, 20, 10, 0
Save data rate: 5 s
Total flow rate: 200 sccm
Post experimental total flow: 200 sscm

### Polarized Light Microscopy (PLM)

Light microscopy was performed using a Polarizing Microscope ECLIPSE LV100POL (Nikon, JPN).

### HPLC Method

HPLC methods for stability and solubility testing are listed in Table 22 and Table 23.

TABLE 22
HPLC Method for Stability Testing

| Instrument | Agilent HPLC 1260 series | |
|---|---|---|
| Column | Eclipse XDB C18, | |
| | 4.6*100 mm, 5 μm | |
| Mobile Phase | A: 0.1% FA in water, | |
| | B: 0.1% FA in ACN | |
| Gradient (T/B %) | 0/20%, 12/40%, | |
| | 15/100%, 16/100% | |
| Column Temperature | 40° | C. |
| Wavelength | 266 | nm |
| Flow Rate | 1.0 | mL/min |
| Injection Conc. | 0.3 | mg/mL |
| Injection Volume | 5 | μL |
| Run Time | 16 | min |
| Post Time | 3 | min |

| | |
|---|---|
| Diluent | ACN/water (1:1) |

TABLE 23
HPLC Method for Solubility Testing

| Instrument | Agilent HPLC 1260 series | |
|---|---|---|
| Column | XDB C18, 4.6*50 mm, 1.8 μm | |
| Mobile Phase | A: 0.1% TFA in water, | |
| | B: 0.1% TFA in ACN | |
| Gradient (T/B %) | 0/20%, 4/70%, | |
| | 4.01/100%, 5/100% | |
| Column Temperature | 40° | C. |
| Wavelength | 266 | nm |
| Flow Rate | 1.0 | mL/min |
| Injection Conc. | 0.25 | mg/mL |
| Injection Volume | 4 | μL |
| Run Time | 5 | min |
| Post Time | 3 | min |

| | |
|---|---|
| Diluent | ACN/water (1:1) |

### Example 1: Experimental Procedures and Preparation of Crystalline Forms of Compound (I)

### General Procedure 1: Estimated Solubility Measurement

Preliminary solubility of Compound (I) was estimated in 13 selected solvents at room temperature. About 2 mg of Compound (I) was weighed into sample vials and different solvents were added with vortex, 20-200 μL per time, until the drug solution was clear by observation. The volume of solvent was up to 1000 V. Then, the estimated solubility (mg/mL) was calculated.

### Results:

Compound (I) appeared to be soluble only in DCM (>120 mg/mL) and THF (~20 mg/mL), and slightly soluble (1-7 mg/mL) in MeOH, MEK, EtOH and ACN. In other selected solvents, the compound was insoluble (<1 mg/mL).

### General Procedure 2: Cooling Crystallization

Cooling crystallization was carried out in 8 solvents from 50° C. to RT. Appropriate amounts of Compound (I) was added into different solvents at 50° C. with stirring to obtain the supersaturated solutions. Then the hot filtrates were cooled from 50° C. to RT gradually. Any obtained solids were characterized accordingly.

### Results:

As summarized in Table 24, Form I was obtained from EtOH, IPA, MEK and EA, and Form II was obtained from ACN and acetone. Form IV and V were obtained in MeOH and toluene, respectively. In addition, the XRPD pattern of solids in acetone was Form II with some missed peaks due to the preferred orientation caused by its needle-shaped morphology. The XRPD pattern of the sample after grinding slightly and the DSC profile were same as Form II.

TABLE 24
Results of Cooling Crystallization

| Sample | Solvent | XRPD Result |
|---|---|---|
| 1 | MeOH | Form IV |
| 2 | EtOH | Form I |
| 3 | IPA | Form I |
| 4 | MEK | Form I |
| 5 | Acetone | Form II with some |
| | | missed peaks |
| 6 | EA | Form I |
| 7 | ACN | Form II |
| 8 | Toluene | Form V |

### General Procedure 3: Anti-Solvent Addition

Anti-solvent crystallization was performed in different DCM or THF systems at RT. An amount of Compound (I) was dissolved in DCM at about 90 mg/mL and in THF at about 17 mg/mL, respectively. The drug solutions were filtered and the filtrates were distributed into sample vials. Each vial contained 150 μL or 0.7 mL of drug solution. Subsequently, anti-solvents were added gradually, 0.15-0.7 mL per time, until many solids precipitated. If precipitation occurred, products were characterized accordingly.

### Results:

All results were given in Table 25. Form I was obtained in most of experiments, while Form IV was obtained from DCM/n-heptane. The XRPD pattern of solids obtained in THF/water was similar as Form III with an extra peak at 5.2 °2θ, which might be due to the residual solvent, and converted to Form III after drying.

TABLE 25
Results of Anti-solvent Precipitation.

| Sample | Solvent | Anti-Solvent | Vₛₒₗᵥₑₙₜ/Vₐₙₜᵢₛₒₗᵥₑₙₜ | XRPD Result |
|---|---|---|---|---|
| A1 | DCM | ACN | 1/9 | Form I |
| A2 | | EA | 1/15 | Form I |
| A3 | | Acetone | 1/7 | Form I |
| A4 | | Toluene | 1/15 | Form I |
| A5 | | IPAC | 1/10 | Form I |
| A6 | | MTBE | 1/5 | Form I |
| A7 | | n-Heptane | 1/5 | Form IV |
| B1 | THF | ACN | 1/20 | No solid |
| B2 | | EA | 1/20 | No solid |
| B3 | | Acetone | 1/20 | No solid |
| B4 | | Toluene | 1/20 | No solid |
| B5 | | IPAC | 1/20 | Form I |
| B6 | | MTBE | 1/20 | Form I |
| B7 | | n-Heptane | 1/20 | Form I |
| B8 | | Water | 1/5 | Form III with extra |
| | | | | peak at 5.2° 2θ |

### General Procedure 4: Slurry in Single Solvent

Appropriate amounts of Compound (I) were weighed into sample vials and the solvent was added to make a suspension respectively. All suspensions were stirred at RT for 5 days and 50° C. for 1 day. Then, the solid was filtered and the filter cake was analyzed by XRPD.

### Results:

As summarized in Table 26, Form I was obtained from MEK, EA, IPAC, MTBE at RT, and from EA and n-heptane at 50° C. Form II was obtained from most of solvents at 50° C., and in THF, ACN and acetone at RT. Pure Form IV was obtained in alcohols at RT, and Form V was obtained in toluene at RT and 50° C.

TABLE 26
Results of Slurry Studies in Single Solvent

| Solvent | Slurry at RT (5 d) | Slurry at 50° C. (1 d) |
|---|---|---|
| THF | Form II | Form II |
| MeOH | Form IV | Form II |
| MEK | Form I | Form II |
| EtOH | Form IV | Form II |
| ACN | Form II | Form II |
| EA | Form I | Form I |
| Acetone | Form II | Form II |
| IPA | Form IV | Form II |
| n-Heptane | Form I + Form IV | Form I |
| IBA | Form II + Form IV | Form II |
| Toluene | Form V | Form V |
| IPAC | Form I | Form I |
| MTBE | Form I | Form I |
| Water | Form III | Form III + Form IV |

### General Procedure 5: Slurry in Mixed Solvent

Appropriate amounts of Compound (I) were weighed into sample vials and the mixed organic solvents/water (1/5) were added to make a suspension respectively. All suspensions were stirred at RT and 50° C. for 3 days. Then, the solid was filtered and the filter cake was analyzed by XRPD.

### Results:

All results were summarized in Table 27. Form III was obtained from all experiments at RT. At 50° C., pure Form VI was obtained from MeOH/water (1/5) and ACN/water (1/5), and the mixtures of Form VI and another form were obtained in other systems.

TABLE 27
Results of Slurry Studies in Mixed Solvent

| Solvent | Slurry at RT (3 d) | Slurry at 50° C. (3 d) |
|---|---|---|
| THF/water (1/5) | Form III | Form IV + Form VI |
| MeOH/water (1/5) | Form III | Form VI |
| MEK/water (1/5) | Form III | Form VI + impurity |
| ACN/water (1/5) | Form III | Form VI |
| EtOH/water (1/5) | Form III | Form IV + Form VI |
| Acetone/water (1/5) | Form III | Form VI + Form III |

### General Procedure 6: Slow Evaporation

Slow evaporation study was carried out in 96-well plate with binary solvents. Appropriate amounts of Compound (I) were added into 13 solvents to obtain the saturated solutions at RT. All filtrates were distributed in 96-well plates according to the solvent matrix in Table 28. Each well contained two different filtrates and the volume of each filtrate was 100 μL. The plate was covered by sealing film with pin holes and allowed to evaporate in an operating laboratory fume hood under ambient conditions. All solids with sufficient quantity were analyzed by XRPD.

TABLE 28
The Solvent Matrix of 96-well Plate

| No. | Solvent |
|---|---|
| 1 | MeOH |
| 2 | ETOH |
| 3 | IPA |
| 4 | DCM |
| 5 | MEK |
| 6 | THF |
| 7 | ACN |
| 8 | MTBE |
| 9 | Acetone |
| 10 | Water |
| 11 | Toluene |
| 12 | EA |
| 13 | IPAC |

| | 1 | 2 | 3 | 4 | 5 | 6 | 7 | 8 | 9 | 10 | 11 | 12 |
|---|---|---|---|---|---|---|---|---|---|---|---|---|
| A | 1 + 1 | 1 + 2 | 1 + 3 | 1 + 4 | 1 + 5 | 1 + 6 | 1 + 7 | 1 + 8 | 1 + 9 | 1 + 10 | 1 + 11 | 1 + 12 |
| B | 1 + 13 | 2 + 2 | 2 + 3 | 2 + 4 | 2 + 5 | 2 + 6 | 2 + 7 | 2 + 8 | 2 + 9 | 2 + 10 | 2 + 11 | 2 + 12 |
| C | 2 + 13 | 3 + 3 | 3 + 4 | 3 + 5 | 3 + 6 | 3 + 7 | 3 + 8 | 3 + 9 | 3 + 10 | 3 + 11 | 3 + 12 | 3 + 13 |
| D | 4 + 4 | 4 + 5 | 4 + 6 | 4 + 7 | 4 + 8 | 4 + 9 | 4 + 10 | 4 + 11 | 4 + 12 | 4 + 13 | 5 + 5 | 5 + 6 |
| E | 5 + 7 | 5 + 8 | 5 + 9 | 5 + 10 | 5 + 11 | 5 + 12 | 5 + 13 | 6 + 6 | 6 + 7 | 6 + 8 | 6 + 9 | 6 + 10 |
| F | 6 + 11 | 6 + 12 | 6 + 13 | 7 + 7 | 7 + 8 | 7 + 9 | 7 + 10 | 7 + 11 | 7 + 12 | 7 + 13 | 8 + 8 | 8 + 9 |
| G | 8 + 10 | 8 + 11 | 8 + 12 | 8 + 13 | 9 + 9 | 9 + 10 | 9 + 11 | 9 + 12 | 9 + 13 | 10 + 10 | 10 + 11 | 10 + 12 |
| H | 10 + 13 | 11 + 11 | 11 + 12 | 11 + 13 | 12 + 12 | 12 + 13 | 13 + 13 | | | | | |

### Results:

Form I, II, III and IV were obtained from different binary solvents by slow evaporation, respectively. All results are summarized in Table 29. In other wells, samples obtained were weakly crystalline or too few to be analyzed.

TABLE 29
Results of Slow Evaporation crystallization

| | Binary | XRPD | | Binary | XRPD |
|---|---|---|---|---|---|
| Well | Solvent | Result | Well | Solvent | Result |
| D2 | DCM/MEK | Form I + IV | B4 | EtOH/DCM | Form IV |
| D3 | DCM/THF | Form I + IV | C5 | IPA/THF | Form II |
| D4 | DCM/ACN | Form I | D9 | DCM/EA | Form II |
| D6 | DCM/Acetone | Form I + IV | D12 | THF/MEK | Form II |
| D10 | DCM/IPAC | Form I + IV | E8 | THF/THF | Form II |
| E10 | THF/MTBE | Form I | E9 | THF/ACN | Form II |
| E11 | THF/Acetone | Form I | F10 | ACN/IPAC | Form II |
| F1 | THF/Toluene | Form I + II | A10 | MeOH/water | Form III |
| F2 | THF/EA | Form I | E12 | THF/water | Form III |
| F3 | THE/IPAC | Form I | B6 | EtOH/THF | Form I + IV |

### Characterization and Definition of Forms

In polymorph screening of Compound (I), seven total forms were identified including five anhydrates (Forms I, II, III, IV, and VII), a hemi-hydrate (Form VI) and a toluene solvate with 1 mol of toluene (Form V). Therein, Form III is converted from a metastable hydrate after air dry for less than one hour. All XRPD patterns and representative peaks were given in FIGS. 1-7 and Tables 1-7.

### Form I

Form I was initially found in the salt screening from slurry experiments in IPA and EA at 50° C., and recovered from several crystallization experiments in many solvents, mainly from cooling and anti-solvent crystallization. The XRPD pattern was named as Pattern 1 (FIG. 1A). Form I was an anhydrous form with relatively high crystallinity and non-hygroscopicity. The melting point was the highest one among all crystal forms at 212° C. (FIG. 1B). Based on DSC data, other four anhydrates (Form II, III, IV and VII) converted to Form I after melting.

### Form H

Form II was also initially found in the salt screening by slurry experiments in ACN, acetone and MEK at 50° C., and obtained from slurry experiments in many solvents at 50° C., and slow evaporation in some binary solvents during salt screening. Besides, Form II was also obtained from cooling crystallization in acetone, ACN or slurry in THF, ACN and acetone at RT. The XRPD pattern was assigned as Pattern 2 (FIG. 2A). Form II was an anhydrous form with high crystallinity, high melting point of 191° C. and slight hygroscopicity (FIG. 2B). During heating by DSC, Form II could convert to Form I after melting. The Form II sample was obtained from slurry in ACN at 50° C. and contained about 0.18% residual solvent.

### Form III

Form III was initially identified in the salt screening during solubility test of Form I in water at 37° C., and characterized by XRPD (FIG. 3A). Form I gradually converted to a metastable hydrate (wet cake) in water after 0.5 hours, and the metastable hydrate is transformed to Form III after air dry for less than 1 hour. In polymorph screening, Form III can obtain from slurry or evaporation experiments in solvents containing water at RT.

Form III was the dehydrated form of a metastable hydrate. Form III had relatively weak crystallinity and high melting point of 196° C. (FIG. 3B). During heating by DSC, Form III converted to Form I after melting.

### Form IV

Form IV was obtained from a few experiments, including cooling crystallization in MeOH, anti-solvent precipitation in DCM/n-heptane, evaporation from EtOH/DCM and slurry in MeOH, EtOH and IPA at RT. The sample obtained from cooling crystallization in MeOH was characterized (FIG. 4A). No residual solvent was detected by NMR, but 0.6% of weight loss in two steps were observed in TGA, might be due to loss of adsorbed water. The melting peak was observed at 143° C. followed by the crystal form transition (FIG. 4B). Form IV was an anhydrous form, and converted to Form I after melting during heating by DSC.

### Form V

Form V was only identified from cooling crystallization or slurry in toluene, and the XRPD pattern is shown in FIG. 5A. Thermal analysis showed Form V was a solvate of toluene with 1 mol toluene (FIG. 5B). After drying at 50° C. overnight, Form V converted to a similar pattern with an extra peak at 5.8° 2θ, which might be a mixture of Form V and III. After being heated to 170° C. by DSC, Form V converted to a mixture of Form I and III.

### Form VI

Form VI was obtained from slurry experiments performed in MeOH/water (1/5) and ACN/water (1/5) at 50° C., and the XRPD is shown in FIG. 6A. The sample was irregular shaped crystals. No residual solvent was detected by NMR, but about 2% of weight loss at RT—110° C. was observed in TGA, due to loss of water. It dehydrated at about 44-75° C., and the melting peak was observed at 185° C. followed by crystal form transition, as shown in FIG. 6B. Form VI was a hemi-hydrate (theoretical water content 1.84%), and converted to Form VII after heated to 150° C. by DSC.

### Form VII

Form VII was found by heating Form VI to 150° C. by DSC, and the XRPD pattern is shown in (FIG. 7A). Form VII was an anhydrous form with weak crystallinity, high melting point of 184° C., and converted to Form I after melting during heating by DSC (FIG. 7B).

Above all, seven forms including one solvate, one hemi-hydrate and five anhydrates of Compound (I) were identified, as summarized in Table 30.

TABLE 30
Characterization Results of Different Forms

| | DSC Onset | TGA | |
|---|---|---|---|
| Form Solvation | (° C.) (ΔH, J/g) | % wt loss/@T | Comments |
| Form I | 212 (91) | 0.56/150 - 229° C. | 0.5% of residual EA |
| (anhydrate) | | | |
| Form II | 191 (26), | 0.18/120 - 200° C. | Converts to Form I |
| (anhydrate) | 196 (exo, 8), | | during heating by DSC |
| | 213 (82) | | |
| Form III | 196 (77) | 0.05/RT - 190° C. | Obtained by drying of wet cake of |
| (anhydrate) | 200 (exo, 16) | | metastable hydrate, Converts to |
| | 211 (25) | | Form I during heating by DSC |
| Form IV | 143 (40) | 0.4/RT - 108° C. | Converted to Form I during |
| (anhydrate) | 152 (exo, 55) | 0.2/108 - 188° C. | heating by DSC |
| | 212 (95) | | |
| Form V (solvate | 100 (91) | 15/RT - 127° C. | Converted to mixture of Form I |
| of toluene) | 187 (0.8) | | and III after desolvation |
| | 195 (exo, 0.6) | | |
| | 212 (79) | | |
| Form VI (hemi- | 44 (47) | 2/RT - 110° C. | Converted to Form VII after |
| hydrate) | 185 (18) | | heating to 150° C. by DSC |
| | 194 (exo, 14) | | |
| | 210 (74) | | |
| Form VII | 184 (13) | / | Converted to Form I during |
| (anhydrate) | 192 (exo, 9) | | heating by DSC |
| | 208 (57) | | |

### Example 2: Interconversion Studies of Polymorph Forms of Compound (I)

Inter-conversion study was performed by competitive slurry of mixed different forms in solvents. The concentration of all slurry suspensions was 20 mg/mL.

### General Procedure 7: Interconversion Study of Form I, II, III and IV

Equal amount of Form I, II, III and IV were added into MeOH, IPA, EA, ACN and water to make suspensions, respectively. All suspensions were stirred at RT and 50° C. The filter cakes were checked by XRPD.

### General Procedure 8: Interconversion Study of Form I and II at 60° C.

Equal amount of Form I and II were added into the saturated solution of Compound (I). The suspensions were stirred in MeOH, IPA and EA at 60° C. overnight. The filter cakes were checked by XRPD.

### General Procedure 9: Interconversion Study of Form II and VII

Equal amount of Form II and VII were added into the saturated solution of Compound (I). The suspensions were stirred in IPA and EA at RT and 50° C. The filter cakes were checked by XRPD.

### General Procedure 10: Interconversion Study of Form II and VI

Equal amount of Form II and VI were slurred in different water activity system (1 mL of EtOH/water) at RT for 3 days. Solid samples were collected by filtration and analyzed by XRPD.

General Procedure 11: Interconversion study of Form III and VI

Equal amount of Form III and VI were slurred in water at RT and 50° C. Solid samples were collected by filtration and analyzed by XRPD.

### Results of Interconversion Studies

Competitive slurry of Form I, II, III and IV, Form II and VII, along with Form I and II, in non-aqueous solvent showed that all mixtures transformed to Form II at RT and 50° C. or 60° C. So, Form II was the most stable anhydrous form at the temperature below 60° C. in non-aqueous solvent. Based on DSC data, Form I had higher melting point than Form II and Form II transformed to Form I during heating by DSC suggesting Form I and Form II should be enantiotropically related. Form I was the more stable anhydrate at high temperature, and the transition temperature of Form I and II was higher than 60° C.

In water, Form II could transform to Form III at RT and to the mixture of Form II and III at 50° C. Form III is converted from the metastable hydrate after air drying for less than one hour. Hence, in water, an unstable hydrate is the more stable form at RT, and it dehydrated to Form III after air drying. The mixture of Form II and III is obtained after drying at 50° C. for 6 days likely due to similar solubility of two forms in water at 50° C., which led to the slow conversion.

Additionally, competitive slurry of Form II and hemi-hydrate Form VI was performed in EtOH/water with different water content at RT. The results showed Form II was physically stable in aqueous solvent with less than 5% of water, but transformed to the mixture of Form II and III in EtOH/50% water.

Besides, competitive slurry of Form III and Form VI was performed in water at RT and 50° C., respectively. The mixture transformed to Form III at RT, but to Form VI at 50° C. Hence, in water, the more stable form was a metastable hydrate at RT, but Form VI at 50° C.

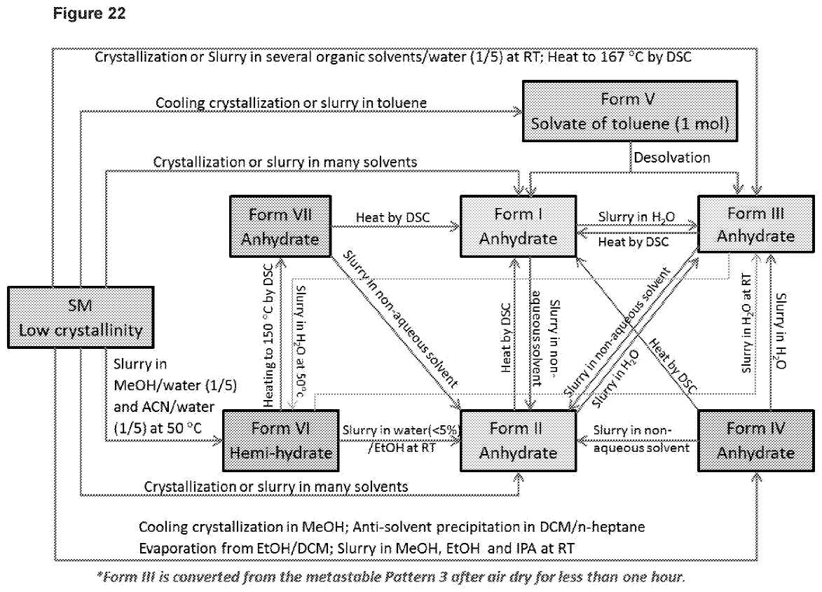

Based on the data collected, the conversion behavior was proposed in FIG. 22.

Among the anhydrous forms, Form II was the more stable form and recommended for further development. Furthermore, slurry and evaporation crystallization are not preferred for manufacture. Besides, since Compound (I) showed poor solubility in most of organic solvents, the solvents available for crystallization are also limited. Hence, the crystallization process development of Form II was recommended.

### Example 3: Stability Studies of Crystalline Form II of Compound (I)

Form II was prepared, characterized, and used for stability study. Solid stability of Form II was evaluated at RT/92.5% RH, 60° C. and 40° C./75% RH conditions for 10 days, respectively.

### Preparation of Form II

About 200 mg of Compound (I) was added into 25 V of ACN to obtain a suspension. The suspension was kept stirring at RT for 16 hours, and then 20 mg of Form II of Compound (I) was added as seeds. After 6.5 hours, solids were collected by filtration and characterized after drying under vacuum at 50° C. overnight.

Form I was initially obtained after 16 hours, and converted to Form II after 6.5 hours with addition of seeds. Form II obtained showed 0.17% of weight loss at 96-200° C. by TGA which was due to loss of residual ACN (FIG. 2B). DVS plot showed Form II was slightly hygroscopic and the adsorption/desorption curve was nearly reversible. It absorbed ~0.5% water at 0-90% RH and the crystal form remained unchanged after DVS testing.

### Physical Stability at RT/92.5% RH

Appropriate amount of Form II was open put at RT/92.5% RH condition for 10 days. Then, the sample was checked by XRPD. No form change was observed. Hence, Form II was physically stable upon high humidity condition for 10 days.

### Stability at 40° C./75% RH and 60° C. Conditions

An amount of Form II was open placed at 60° C. and 40° C./75% RH up to 10 days. At 0, 3 and 10 day, the sample was dissolved in diluent to prepare solution for purity analysis by HPLC. Solid samples were analyzed by XRPD to check the crystal form. The results were summarized in Table 31. No obvious degradation occurred and no form changed under testing conditions. Form II was physically and chemically stable at 40° C./75% RH and 60° C. for 10 days.

TABLE 31
Stability Evaluation Results

| | | 40° C./75% RH | | 60° C. | |
|---|---|---|---|---|---|
| | | Purity | | Purity | |
| Sample | Time | (Area %) | XRPD | (Area %) | XRPD |
| Form II | 0 d | 99.17 | / | 99.17 | / |
| | 3 d | 99.15 | No | 99.15 | No |
| | | | change | | change |
| | 10 d | 99.13 | No | 99.09 | No |
| | | | change | | change |

### Mechanical Treatment

Form II (~15 mg) was ground into a mortar for about 5 minutes, and then checked by XRPD at 2 min and 5 min, respectively. The crystal form remained unchanged after grinding.

### CONCLUSION

Polymorph screening of Compound (I) free base was carried out. Total seven forms and one metastable pattern were identified including five anhydrates (Form I, II, III, IV and VII), a hemi-hydrate (Form VI), a solvate of toluene with 1 mol (Form V) and unstable hydrous Pattern 3.

Among all forms, Form II was the most stable anhydrous form at the temperature below 60° C. in non-aqueous solvent, and physically stable in aqueous solvent with less than 5% of water. In water, anhydrate Form III, after drying of wet cake of Pattern 3, was obtained at RT, and the hemi-hydrate Form VI was more stable at 50° C. Form I and Form II are enantiotropically related. Form I was the more stable anhydrate at high temperature, but the transition temperature of Form I and II was higher than 60° C.

Overall, Form II was the more stable anhydrous form with favorable solid state properties.

### Example 4: Experimental Procedures and Preparation of Crystalline Salt Forms of Compound (I)

### Salt Screening in 96-Well Plate

Appropriate amounts of 12 acids were dissolved in MeOH to prepare an acid solution with concentration of 0.1 M. About 360 mg of Compound (I) was dissolved in 24 mL of MeOH/DCM (1/1) at RT to prepare the drug solution at 15 mg/mL. The drug solution was distributed into 96-well plate. Each well contained 200 μL of drug solution. 1.1 eq. of each acid solution was added, according to the acid/solvent matrix in Table 32. The wells evaporated to dryness, and then 200 μL of selected solvents were added into each well. Wells were covered with a film with pinhole, and evaporated under ambient conditions. After evaporation to dryness, states of samples in 96-well plate were given in Table 32. Most of solids were crystals determined by PLM, but some crystals analyzed by XRPD had low crystallinity. The samples in row E were analyzed by ¹H-NMR. The results showed that free base formed salt with all selected acids, due to the obvious chemical shifts.

TABLE 32
Results of 96-Well screening plate.

| | | | | | | | | | 8 | | | 11 | |
|---|---|---|---|---|---|---|---|---|---|---|---|---|---|
| | | | | | | | | 7 | L- | 9 | 10 | L- | 12 |
| | | 1 | 2 | 3 | 4 | 5 | 6 | Maleic | Tartaric | Fumaric | Citric | Malic | Succinic |
| | | HCl | HBr | H₂SO₄ | TsOH | MsOH | H₃PO₄ | acid | acid | acid | acid | acid | acid |
| A | MeOH | WCR* | CR | CR | WCR* | WCR* | Glassy | WCR* | AM* | AM* | WCR* | WCR* | WCR* |
| B | IPA | Glassy | CR | Glassy | Glassy | Glassy | Glassy | WCR* | AM* | WCR* | AM | AM | CR |
| C | THF | CR | CR | CR | Glassy | CR | CR | CR | CR | CR | CR | AM | CR |
| D | ACN | CR | CR | CR | Glassy | Glassy | Glassy | CR | CR | CR | AM | CR | CR |
| E | MTBE | CR | CR | CR | Glassy | Glassy | Glassy | Glassy | CR | CR | AM | CR | CR |
| F | Acetone | CR | CR | CR | CR | CR | Glassy | CR | CR | CR | CR | CR | CR |
| G | Water | CR | CR | CR | CR | CR | CR | CR | CR | CR | CR | CR | CR |
| H | EA | CR | WCR* | CR | CR | CR | WCR* | CR | CR | CR | CR | CR | CR |

CR = Crystalline,
WCR = Weak Crystalline,
AM = Amorphous;
*represents the sample was analyzed by XRPD.
Others were observed under PLM.

### General Procedure 12: Preparation of Salts

All salts were tried to prepare by salt-forming reactions in lab scale (~23 mg, 0.05 mmol). The free base was dissolved in different solvents at room temperature (RT) or 50° C., and then acid was added to form salt. For all reactions, the drug solutions were a suspension before adding acid. All crystalline salts were directly precipitated and characterized after drying under vacuum at 50° C. for 4-16 hours. If no precipitation occurred, various crystallization methods were attempted to prepare crystalline salts, including cooling crystallization, evaporation and anti-solvent precipitation. Solids were collected by filtration, dried under vacuum and analyzed by XRPD.

### Hydrochloride Salts of Compound (I)

Several solvents and methods were used for the preparation of crystalline HCl salts. Except in IPA and EA, crystalline HCl salts were obtained in other solvents by reaction crystallization. Total four crystalline HCl salt forms of Compound (I) were identified, and assigned as Form 1 to 4, respectively. Forms I, II, III, and IV were further characterized by DSC, TGA and ¹H-NMR. The detailed information and results were summarized in Table 33.

TABLE 33
Preparation of Hydrochloride Salt Forms of Compound (I)

| Solvent | Temp. | HCl (eq.) | Crystallization Procedure | XRPD Result |
|---|---|---|---|---|
| ACN | 50° C. | 1M in ACN | Jelly appeared quickly after adding HCl and | Form I |
| | | (1.1 eq) | converted to crystals after being slurried | (FIG. 10A) |
| | | | at 50 ° C. for about 4.5 h. | |
| ACN/H₂O | 50° C. | Added | Sticky solids appeared quickly after adding | Form II |
| (9/1) | | directly | HCl and converted to crystals after being | (FIG. 11A) |
| | | (1.1 eq) | slurried at 50° C. for about 4.5 h. | |
| ACN | 50° C. | 1M in ACN | Sticky solids appeared quickly after adding | Form III |
| | | (2.05 eq) | HCl and converted to crystals after being | (FIG. 12A) |
| | | | slurried at 50° C. for about 6 h. | |
| MeOH | RT | Added | Precipitation occurred after about 5 h | Form IV |
| | | directly | and crystals were obtained after being | (FIG. 13A) |
| | | (2.05 eq) | slurried at RT overnight. | |

HCl salt Form I was an anhydrate with high melting point of 244° C. (FIG. 10B). However, the sample was hygroscopic and the DVS plot was unreversible. It absorbed ~1.5% water at 0-70% RH, while water uptake significantly increased from 2.6% to 19% at 80-90% RH, and the water uptake at 80-90% RH didn't reach equilibrium, suggesting it would adsorb more water at high humidity. Hence, HCl salt Form I should be stored at the humidity lower than 70%.

HCl Form III was an anhydrate containing residual ACN, three endothermic peaks were observed in DSC (FIG. 12B), likely due to desolvation and melting, respectively.

HCl salt Form II and Form IV was di-hydrate and mono-hydrate, respectively. DSC results (FIGS. 11B and 13B) showed temperatures of dehydration were both low, suggesting two hydrates might be unstable.

### Hydrobromide Salt Form I

ACN and Acetone were used as solvents to prepare HBr salts of Compound (I). Crystalline HBr salt was obtained in ACN and assigned as Form 1 (FIG. 14A), but sticky sample was obtained in acetone. Form 1 had relatively weak crystallinity. No residual solvent was detected by NMR, suggesting the first weight loss by TGA (FIG. 14B) was due to loss of water, corresponding to the broad endothermic peak by DSC. HBr salt Form I might be an unstable hydrate considering the low temperature of dehydration.

### Sulfate Salt Form I

Crystalline sulfates were obtained by salt-forming reaction of the free base with 0.55 eq. and 1.1 eq. of H₂SO₄, respectively. However, only one XRPD pattern was identified from the reaction in ACN with 1.1 eq. of H₂SO₄, and assigned as Form I (FIG. 15A). Sulfate Form I was irregular shaped crystals. No residual solvent was detected by ¹H-NMR, but 0.2% of weight loss at 60-150° C. was observed by TGA (FIG. 15B), which might be due to loss of water. A small and wide endothermic peak due to dehydration followed by a melting/decomposition peak were observed by DSC. Sulfate Form I may be an anhydrate.

### Tosylate Salt Form I

Crystalline tosylate was obtained from MeOH at RT and 50° C., and assigned as Form I (FIG. 9A). Tosylate Form 1 was an anhydrate with relatively high crystallinity, high melting point of 216° C. (FIG. 9B). The sample was hygroscopic and the DVS plot was nearly reversible. It absorbed ~3.3% water at 0-80% RH. The crystal form remained unchanged after DVS testing. Tosylate Form I was selected as the lead salt for solubility and stability study.

### Phosphate Salt Form I

Crystalline phosphate was obtained from ACN and acetone, and assigned as phosphate salt Form I. Form I was analyzed by XRPD and DSC. (FIGS. 16A and 16B) Phosphate Form I was an anhydrate, but the crystallinity was relatively weak. In addition, based on the phenomenon of experiments, phosphate Form I had very low solubility in organic solvents, even in DMSO, which has risk for further development.

### Maleate Salt Form I

Maleate salt Form I was prepared using EA and ACN as solvent and identified as an anhydrate. Maleate Form I had high crystallinity and melting at 171° C. The characterized sample, obtained in EA, was needle shaped crystals. The crystals were characterized by XRPD (FIG. 8A). No residual solvent was detected by ¹H-NMR, while 0.4% of weight loss at RT—80° C. was observed in TGA, might be due to loss of adsorbed water (FIG. 8B). Form I was hygroscopic and the DVS plot was reversible. It absorbed ~2.5% water at 0-80% RH. The crystal form remained unchanged after DVS testing. In addition, physical stability of maleate Form I was evaluated under high humidity (RT/92.5% RH) for 18 hours. The solid state and crystal form remained unchanged. Maleate Form I was selected as the lead salt for solubility and stability study.

### Tartrate Salt Form I

Tartrate Form 1 was obtained from the reaction in ACN at 50° C. Based on the characterization data in FIGS. 17A and 17B, tartrate Form I might be a solvate or hydrate and might be unstable as the temperature of desolvation or dehydration was low.

### Fumarate Salt Forms I and II

Five solvents were used to prepare crystalline fumarate. Form I was prepared from slurries in ACN and MEK at 50° C., while Form II was prepared from a slurry in IPA at 50° C. Both fumarate Form I and Form II were anhydrates containing much residual solvents, which might be a potential issue in the manufacture. XRPD, DSC, and TGA is described in FIGS. 18A, 18B, 19A and 19B.

### Malate Salt Form I

MeOH, ACN and acetone were used for prepare crystalline malate, respectively. Malate Form I was obtained from acetone, but two weakly crystalline patterns were obtained from MeOH and ACN. Malate salt Form I contained about 0.03% of residual acetone by ¹H-NMR, suggesting the 0.3% of weight loss before 75° C. by TGA might be due to loss of solvent and adsorbed water (FIG. 20A). In addition, one broad endothermic peak due to dehydration and two overlapped endothermic peaks due to melting and decomposition were observed in DSC. XRPD data is described in FIG. 20B.

### Succinate Salt Form I

Succinate salt Form I was prepared in acetone and obtained after slurry at 50° C. Form I was needle shaped crystals. XRPD data is described in FIG. 21A. No residual solvent and no weight loss were detected by NMR and TGA (FIG. 21B). The melting peak appeared at 160-162° C. by DSC. The sample was an anhydrate. In addition, physical stability of succinate Form I was evaluated under high humidity (RT/92.5% RH) for 18 hours. The solid state changed from powder to be sticky, and the crystal form converted to a new form with weak crystallinity.

### Example 5: Solubility Studies of Maleate salt Form I and Tosylate salt Form I of Compound (I)

### Preparation of Maleate Salt Form I

About 235 mg of Compound (I) was added into 30V of EA at RT to obtain a suspension. Then, 61.18 mg of maleic acid (1.05 eq.) was dissolved in 1 mL of MeOH and added. Sticky solids appeared immediately and undissolved free base existed. After about 10 min, some flocculation solids appeared. The resulting suspension was kept stirring at 50° C. overnight. Solids were collected by filtration, washed by EA and dried under vacuum at 50° C. overnight. 280 mg of maleate Form I was obtained with yield of 95%, and 0.4% residual EA was detected by NMR.

### Preparation of Tosylate Salt Form I

About 235 mg of Compound (I) was added into 21V of MeOH at RT to obtain a suspension. Then, 90.45 mg of p-TsOH (1.05 eq.) was added. The mixture was clear quickly and precipitation occurred after 5 min. The suspension was kept stirring at RT overnight. Solids were collected by filtration, washed by MeOH and dried under vacuum at 50° C. overnight. 285 mg of tosylate Form I was obtained as a light yellow solid with yield of 89%.

### Solubility Testing

The solubility of maleate Form I, and tosylate Form I were tested in bio-relevant media (SGF, FaSSIF, and FeSSIF) and water at 37° C. up to 24 hours. About 15 mg of the sample was weighed into sample vials and then 3 mL of media was added to make a suspension. All suspensions were shaken at 37° C. with a rate of 200 rpm. At 0.5, 2 and 24 hours, each suspension was filtered and the filtrate was analyzed by HPLC to test the solubility. pH of the filtrate was measured, and the filter cake was analyzed by XRPD.

### Results:

The solubilities of the samples showed obvious pH dependency. Tosylate Form I and maleate Form I possess solubility of about 2 mg/mL and 28 mg/mL, respectively (estimated solubility by visual assessment). Solubility's in SGF were more than 5 mg/mL and the samples have similar solubility in FeSSIF (2-3 mg/mL). In FaSSIF, the solubility of maleate Form I was ~30 folds of that of tosylate Form I. Tosylate Form I remained unchanged in water after 24 hours. Among the samples, maleate Form I had relatively higher solubility than Tosylate Salt Form I.

### Example 6: Stability Studies of Maleate Salt Form I and Tosylate Salt Form I of Compound (I)

Appropriate amounts of maleate Form I and tosylate Form I were placed at 60° C. and 40° C./75% RH for up to 24 days. At 0 and 24 day, the sample was dissolved in diluent to prepare solution for purity analysis by HPLC. Solid samples were analyzed by XRPD to check the crystal form.

### Results:

Tosylate Form I and maleate Form I was physically stable under two conditions. However, the purities both decreased by 0.1% at 40° C./75% RH and 0.5% at 60° C. for 24 days, which mainly due to the increase of elimination product impurity. The data is summarized in Table 34.

TABLE 34
Stability Evalutation Results

| | | 40° C./75% | | | |
|---|---|---|---|---|---|
| | Initial | RH - 24 days | | 60° C. - 24 days | |
| | purity | Purity | | Purity | |
| Sample | (Area %) | (Area %) | XRPD | (Area %) | XRPD |
| Tosylate Form I | 99.32 | 99.20 | No | 98.83 | No |
| | | (↓ 0.12) | change | (↓ 0.49) | change |
| Maleate Form I | 99.08 | 98.97 | | 98.58 | |
| | | (↓ 0.11) | | (↓ 0.50) | |

In salt screening, total 12 salts forms and three free base forms were identified. Therein, tosylate Form I and maleate Form I with better solid state properties were selected as lead salts for solubility.

The solubilities of three samples showed obvious pH dependency. Tosylate Form I and maleate Form I have high solubility in water. They showed similar solubility in SGF (>5 mg/mL) and in FeSSIF (2-3 mg/mL). In FaSSIF, the solubility of maleate Form I was ~30 folds of that of tosylate Form I. Maleate Form I had relatively higher solubility than others. Tosylate Form I and maleate Form I was physically stable under two conditions. However, the purities both decreased by 0.1% at 40° C./75% RH and 0.5% at 60° C. for 24 days.

1. Crystalline Form II of Compound (I): 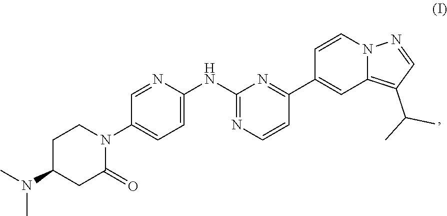 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 4.7°, 5.0°, 8.4°, and 21.8°±0.2 in 2θ.

2. The crystalline Form II of Compound (I) according to claim 1, characterized by an X-ray powder diffraction pattern which comprises peaks at chosen from 4.7°, 5.0°, 8.4°, 13.8°, 19.5°, 21.8°, and 23.2°±0.2 in 2θ.

3. The crystalline Form II of Compound (I) according to claim 1, characterized by an X-ray powder diffraction pattern which comprises peaks at chosen from 4.7°, 5.0°, 8.4°, 9.0°, 11.5°, 13.8°, 19.5°, 21.8°, 23.2°, and 23.5°±0.2 in 2θ.

4. The crystalline Form II of Compound (I) according to any one of claims 1 to 3, characterized by a differential scanning calorimeter (DSC) peak phase transition temperatures of 190.9±2° C., 195.3±2° C., and 210.9±2° C.

5. Crystalline Form I of Compound (I): 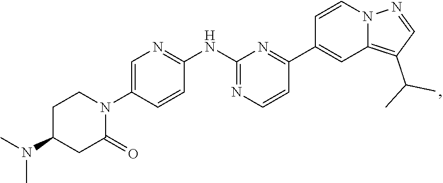 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 6.8°, 18.4°, 21.4°, and 24.1°±0.2 in 2θ.

6. The crystalline Form I of Compound (I) according to claim 5, characterized by a differential scanning calorimeter (DSC) peak phase transition temperature of 211.6±2° C.

7. Crystalline Form III of Compound (I): 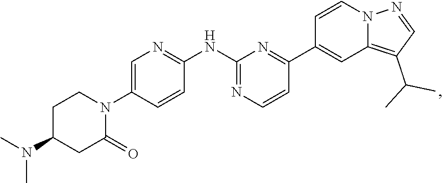 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 5.7°, 7.4°, 10.5°, 14.2°, and 20.5°±0.2 in 2θ.

8. The crystalline Form III of Compound (I) according to claim 7, characterized by an X-ray powder diffraction pattern which comprises peaks at chosen from 5.7°, 7.4°, 10.5°, 14.2°, 17.0°, 20.1°, and 20.5°±0.2 in 2θ.

9. The crystalline Form III of Compound (I) according to claim 7, characterized by an X-ray powder diffraction pattern which comprises peaks at chosen from 5.7°, 7.1°, 7.4°, 10.5°, 11.2°, 14.2°, 17.0°, 20.1°, 20.5°, and 21.1°±0.2 in 2θ.

10. The crystalline Form III of Compound (I) according to any one of claims 7 to 9, characterized by a differential scanning calorimeter (DSC) peak phase transition temperature of 196.4±2° C., 199.6±2° C., and 211.3±2° C.

11. Crystalline Form IV of Compound (I): 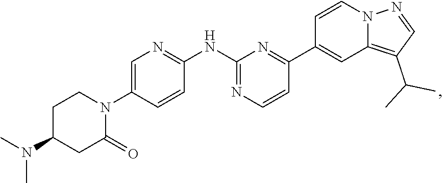 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 3.8°, 5.4°, and 15.8°±0.2 in 2θ.

12. The crystalline Form IV of Compound (I) according to claim 11, characterized by a differential scanning calorimeter (DSC) peak phase transition temperatures of 143.4±2° C., 152.0±2° C., and 211.8±2° C.

13. Crystalline Form V of Compound (I): 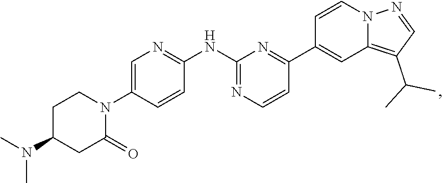 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 8.8°, 11.1°, 20.2°, and 23.7°±0.2 in 2θ.

14. The crystalline Form V of Compound (I) according to claim 13, characterized by an X-ray powder diffraction pattern which comprises peaks at chosen from 6.3°, 8.8°, 11.1°, 18.9°, 20.2°, 23.7°, and 27.9°±0.2 in 2θ.

15. The crystalline Form V of Compound (I) according to claim 13, characterized by an X-ray powder diffraction pattern which comprises peaks at chosen from 6.3°, 8.8°, 11.1°, 12.7°, 16.0°, 18.9°, 20.2°, 22.2°, 23.7°, and 27.9°±0.2 in 2θ.

16. The crystalline Form V of Compound (I) according to any one of claims 13 to 15, characterized by a differential scanning calorimeter (DSC) peak phase transition temperature of 100.3±2° C., 187.4±2° C., 195.2±2° C., and 211.8±2° C.

17. Crystalline Form VI of Compound (I): 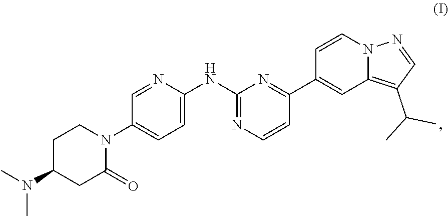 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 5.6°, 9.9°, 13.7°, and 19.3°±0.2 in 2θ.

18. The crystalline Form VI of Compound (I) according to claim 11, characterized by a differential scanning calorimeter (DSC) peak phase transition temperatures of 43.7±2° C., 185.1±2° C., 194.3±2° C., and 210.2±2° C.

19. Crystalline Form VII of Compound (I): 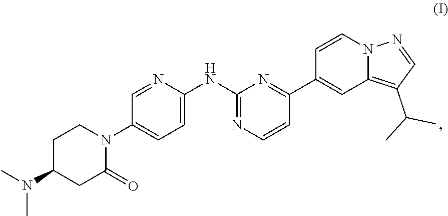 [SMILES: CC(C)c1cnn2ccc(-c3ccnc(Nc4ccc(N5CC[C@H](N(C)C)CC5=O)cn4)n3)cc12]
characterized by an X-ray powder diffraction pattern which comprises peaks at chosen from 5.3°, 5.6°, and 10.2°±0.2 in 2θ.

20. The crystalline Form VII of Compound (I) according to claim 13, characterized by an X-ray powder diffraction pattern which comprises peaks at chosen from 5.3°, 5.6°, 8.2°, 10.2°, 19.3°, and 23.9°±0.2 in 2θ.

21. The crystalline Form VII of Compound (I) according to claim 13, characterized by an X-ray powder diffraction pattern which comprises peaks at chosen from 5.3°, 5.6°, 7.6°, 8.2°, 10.2°, 15.2°, 19.3°, 20.6°, and 23.9°±0.2 in 2θ.

22. The crystalline Form VII of Compound (I) according to any one of claims 13 to 15, characterized by a differential scanning calorimeter (DSC) peak phase transition temperatures of 184.3±2° C., 192.0±2° C., and 207.8±2° C.

23. Crystalline Form I of maleate salt of Compound (I) represented by the following Formula: 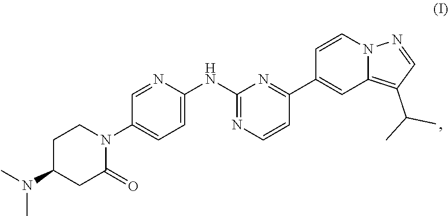 [SMILES: CC(C)c1cnn2ccc(-c3ccnc(Nc4ccc(N5CC[C@H](N(C)C)CC5=O)cn4)n3)cc12]
wherein the molar ratio between Compound (I) and maleic acid is 1:1, characterized by an X-ray powder diffraction pattern which comprises peaks at chosen from 5.7°, 9.3°, 10.3°, 13.9°, and 20.7°±0.2 in 2θ.

24. The crystalline Form I of maleate salt of claim 23, characterized by an X-ray powder diffraction pattern which comprises peaks at 5.7°, 9.3°, 10.3°, 13.9°, 20.7°, 22.4°, 24.1°, 24.5°, and 26.8°±0.2 in 2θ.

25. The crystalline Form I of maleate salt of claim 23 or 24, characterized by a differential scanning calorimeter (DSC) peak phase transition temperature of 173.9±2° C.

26. Crystalline Form I of tosylate salt of Compound (I) represented by the following Formula: 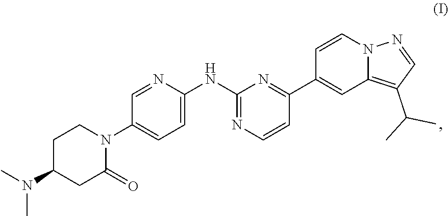 [SMILES: CC(C)c1cnn2ccc(-c3ccnc(Nc4ccc(N5CC[C@H](N(C)C)CC5=O)cn4)n3)cc12]
wherein the molar ratio between Compound (I) and p-toluenesulfonic acid is 1:1, characterized by an X-ray powder diffraction pattern which comprises peaks at chosen from 9.5°, 11.0°, 14.1°, 21.4°, and 21.9°±0.2 in 2θ.

27. The crystalline Form I of tosylate salt of claim 26, characterized by an X-ray powder diffraction pattern which comprises peaks at 7.2°, 9.5°, 11.0°, 14.1°, 21.1°, 21.4°, 21.9°, 22.6°, and 24.6°±0.2 in 2θ.

28. The crystalline Form I of tosylate salt of claim 26 or 27, characterized by a differential scanning calorimeter (DSC) peak phase transition temperatures of 38.9±2° C. and 203.0±2° C.

29. A pharmaceutical composition comprising the compound of any one of claims 1-28, and a pharmaceutically acceptable carrier.

30. A method of treating a cancer comprising administering to a subject in need thereof an effective amount of a compound of any one of claims 1-28, wherein the cancer is selected from the group consisting of colorectal cancer, breast cancer, lung cancer, prostate cancer, glioblastoma, mantel cell lymphoma, chronic myeloid leukemia and acute myeloid leukemia.

31. A method of treating a cancer by inhibiting of a cyclin-dependent kinase (CDK), said method comprising administering to a subject in need thereof an effective amount of a compound of any one of claims 1-28.

32. The method of claim 31, wherein the cancer is carcinoma of the bladder, breast, colon, kidney, epidermis, liver, lung, oesophagus, gall bladder, ovary, pancreas, stomach, cervix, thyroid, nose, head and neck, prostate, or skin; a hematopoietic tumor of lymphoid lineage; a hematopoietic tumor of myeloid lineage; thyroid follicular cancer; a tumor of mesenchymal origin; a tumor of the central or peripheral nervous system; melanoma; seminoma; teratocarcinoma; osteosarcoma; xeroderma pigmentosum; keratoctanthoma; or Kaposi's sarcoma.

33. The method of claim 32, wherein the hematopoietic tumor of lymphoid lineage is leukemia, acute lymphocytic leukemia, chronic lymphocytic leukemia, B-cell lymphoma, T-cell lymphoma, multiple myeloma, Hodgkin's lymphoma, non-Hodgkin's lymphoma, hairy cell lymphoma, or Burkett's lymphoma.

34. The method according to claim 31, wherein the cancer is pRb⁺ breast cancer, or hormone receptor (HR)-positive (e.g., estrogen receptor positive (ER⁺), progesterone receptor positive (PR⁺), or ER⁺PR⁺), HER2/neu-negative cancer.

35. The method of claim 34, wherein the cancer is advanced or metastatic or recurrent breast cancer.

36. The method of claim 35, wherein the breast cancer is in an adult woman, or a postmenopausal woman.

37. The method of any of claims 34-36, further comprising administering a second agent selected from: an aromatase inhibitor, a Selective Estrogen Receptor Modulator (SERM), a pure antiestrogen with no estrogen agonist activity, a compound that temporarily suppresses ovarian function (e.g., estrogen and/or progesterone production) such as a gonadotropin-releasing hormone (GnRH) agonist or a luteinizing hormone-releasing hormone (LH-RH) agonist, a compound that inhibits CCYP3A4, or a monoclonal antibody or an antigen-binding fragment thereof against IGF-1/IGF-2.

38. The method of any of claims 30-37, further comprising administering an immune checkpoint inhibitor (such as a PD-1 inhibitor, a PD-L1 inhibitor, or a CTLA-4 inhibitor), a receptor Tyr kinase inhibitor, and/or an antogonist of hormone receptor (such as estrogen receptor).
